(19) United States
(12) Patent Application Publication
(10) Pub. No.: US 2017/0148240 A1
(43) Pub. Date: May 25, 2017

(54) SCALE-BASED BIOMETRIC AUTHORIZATION OF COMMUNICATION BETWEEN SCALE AND A REMOTE USER-PHYSIOLOGIC DEVICE

(71) Applicant: Physiowave, Inc., Santa Clara, CA (US)

(72) Inventors: Gregory T. Kovacs, Palo Alto, CA (US); Richard M. Wiard, Campbell, CA (US)

(21) Appl. No.: 15/354,861

(22) Filed: Nov. 17, 2016

Related U.S. Application Data
(63) Continuation of application No. PCT/US2016/062484, filed on Nov. 17, 2016.
Application No. PCT/US2016/062484 is a continuation of application No. PCT/US2016/062505, filed on Nov. 17, 2016.
(60) Provisional application No. 62/258,238, filed on Nov. 20, 2015.
(60) Provisional application No. 62/263,385, filed on Dec. 4, 2015.
(60) Provisional application No. 62/266,523, filed on Dec. 11, 2015.

(51) Int. Cl. G07C 9/00; G01G 23/42; G01G 21/22; G01G 19/50; H04L 29/06; A61B 5/1171; A61B 5/1172; A61B 5/117; A61B 5/0402; A61B 5/11; G01G 23/36; A61B 5/00

(52) U.S. Cl. CPC G07C 9/00158; G01G 23/36; G01G 23/42; G01G 21/22; G01G 19/50; A61B 5/742; A61B 5/1176; A61B 5/1172; A61B 5/117; A61B 5/0402; A61B 5/1102; H04L 63/0861; H04L 63/083; H04L 63/0876

(57) ABSTRACT

Aspects of the disclosure are directed to an apparatus including a scale having a platform for a user to stand on, data-procurement circuitry to collect signals indicative of the user's identity and cardio-physiological measurements, processing circuitry, a communication activation circuit, and an output circuit. The processing circuitry processes data obtained by the data-procurement circuitry and therefrom generates cardio-related physiologic data, identifies a scale-based biometric of the user using the collected signals, and validates user data, including data indicative of the user's identity and the generated cardio-related physiologic data, as concerning the user. The communication activation circuit activates communication between the scale and a remote user-physiologic device in response identifying the scale-based biometric and verifying authorization data from the remote user-physiologic device corresponds to the user. The output circuit receives the validated user data and outputs at least a portion of the user data to the remote user-physiologic device.

### RELATED APPLICATION DATA

This application is related to PCT Application (Ser. No. PCT/US2016/062505), entitled “Remote Physiologic Parameter Assessment Methods and Platform Apparatuses”, filed on Nov. 17, 2016, PCT Application (Ser. No. PCT/US2016/062484), entitled “Scale-Based Parameter Acquisition Methods and Apparatuses”, filed on Nov. 17, 2016, U.S. Provisional Application (Ser. No. 62/258,238), entitled “Condition or Treatment Assessment Methods and Platform Apparatuses”, filed Nov. 20, 2015, U.S. Provisional Application (Ser. No. 62/263,385), entitled “Scale-Based Biometric Authorization of Communication Between Scale and A Remote User-Physiologic Device”, filed Dec. 4, 2015, and U.S. Provisional Application (Ser. No. 62/266,523) entitled “Social Grouping Using a User-Specific Scale-Based Enterprise System”, filed Dec. 11, 2015″, which are fully incorporated herein by reference.

### OVERVIEW

Various aspects of the present disclosure are directed toward methods, systems and apparatuses that are useful in authorizing communication between a scale and a user-physiologic device using a scale-based biometric and user-physiologic device-based authorization data.

Various aspects of the present disclosure are directed toward monitoring different physiological characteristics for many different applications. For instance, physiological monitoring instruments are often used to measure a number of patient vital signs, including blood oxygen level, body temperature, respiration rate and electrical activity for electrocardiogram (ECG) or electroencephalogram (EEG) measurements. For ECG measurements, a number of electrocardiograph leads may be connected to a patient's skin, and are used to obtain a signal from the patient.

Obtaining physiological signals (e.g., data) can often require specialty equipment and intervention with medical professionals. For many applications, such requirements may be costly or burdensome. These and other matters have presented challenges to monitoring physiological characteristics.

Aspects of the present disclosure are directed to a platform apparatus that provides various features including communicating with other user devices, such as a remote user-physiologic device, in response to a dual authorization. The platform apparatus, such as a body weight scale, provides various features such as collecting scale-obtained data including a scale-based biometric and cardio-physiological measurements from a user while the user is standing on the platform apparatus and outputting the scale-obtained data to external circuitry in response to verifying the scale-based biometric in addition to authorization data received from the external circuitry. The scale-obtained data can be correlated (e.g., combined) with user-device obtained data and additional processing can be performed on the correlated data sets by the scale and/or user device. The external circuitry provides the features of collecting signals indicative of the user's identity, including the authorization data, and cardio-physiological data, and outputs the authorization data to the scale. By authorizing communication between the platform apparatus and the external circuitry responsive to the scale-based biometric and authorization data from the external circuitry, user sensitive data such as health data is communicated between the devices only when both devices are authorized. In various aspects, the devices are authorized in response to the platform apparatus verifying both devices are being used by the same user.

In various aspects, the scale and the remote-user physiologic device (or other devices) are time synchronized prior to obtaining the user data. As further discussed herein, the scale and remote-user physiologic device can be time synchronized while the user is standing on the scale and/or by tapping the remote-user physiologic device on the scale to time synchronize via the force sensor circuitry (e.g., strain gauges) of the scale and a built-in accelerometer of the remote-user physiologic device.

As used herein, a user device includes processing circuitry and output circuitry to collect various data (e.g., signals) and communicate the data to the scale and/or other circuitry. Example user devices include cellphones, tablets, standalone servers or central processing units, among other devices. A wearable device is a user device (and/or a remote user-physiologic device) that is worn by a user, such as on a user's wrist, head, or chest. Example wearable devices include smartwatches and fitness bands, smartglasses, chest heart monitors, etc. A remote user-physiologic device is a user device (and/or a wearable device) that further includes sensor circuitry or other circuit to collect physiologic data from the user, and, can optionally be in secured communication with the scale or other circuitry. Example remote user-physiological devices include smartwatches or fitness bands that collect heart rate and/or ECG and/or body temperature, medical devices, implanted medical devices, smartbeds, among other devices. Example physiologic data collected by remote user-physiologic devices includes glucose measurements, blood pressure, ECG or other cardio-related data, body temperature, among other data. As used herein, the terms “user device”, “wearable device”, and “remote user-physiologic device” can be interchangeably used, as one of skill may appreciate that in specific examples, a particular device may be considered one or more of a user device, a wearable device, a remote user-physiologic device. As a specific example, a particular remote user-physiologic device is a smartwatch and can be referred to as a wearable device or a user device. In other aspects, the remote user physiologic device may not be a wearable device, such as a medical device that is periodically or temporarily used.

Various aspects of the present disclosure are directed toward multisensory biometric devices, systems and methods. Aspects of the present disclosure include user-interactive platforms, such as scales, large and/or full platform-area or dominating-area displays and related weighing devices, systems, and methods. Additionally, the present disclosure relates to electronic body scales that use impedance-based biometric measurements. Various other aspects of the present disclosure are directed to biometrics measurements such as body composition and cardiovascular information. Impedance measurements are made through the feet to measure fat percentage, muscle mass percentage and body water percentage. Additionally, foot impedance-based cardiovascular measurements are made for an ECG and sensing the properties of blood pulsations in the arteries, also known as impedance plethysmography (IPG), where both techniques are used to quantify heart rate and/or pulse arrival timings (PAT). Cardiovascular IPG measures the change in impedance through the corresponding arteries between the sensing electrode pair segments synchronous to each heartbeat.

In certain embodiments, the present disclosure is directed to apparatuses and methods including a scale and a remote user-physiologic device. The scale includes a user display to display data to a user while the user is standing on the scale, a platform for a user to stand on, data-procurement circuitry, and processing circuitry. The data-procurement circuitry includes force sensor circuitry and a plurality of electrodes integrated with the platform for engaging the user with electrical signals and collecting signals indicative of the user's identity and cardio-physiological measurements while the user is standing on the platform. The processing circuitry includes a CPU and a memory circuit with user-corresponding data stored in the memory circuit. The processing circuitry is arranged with (e.g., electrically integrated with or otherwise in communication) to process data obtained by the data-procurement circuitry while the user is standing on the platform and therefrom generate cardio-related physiologic data corresponding to the collected signals. Further, the processing resource transitions the scale, including the user display, the data-procurement circuitry, and the processing circuitry from a reduced power-consumption mode of operation to at least one higher power-consumption mode of operation responsive to the user standing on the platform. And, the processing resource, in response, identifies a scale-based biometric of the user using the collected signals, and therefrom, validates user data, including data indicative of the user's identity and the generated cardio-related physiologic data, as concerning a specific user associated with the scale-based biometric.

Biometrics, as used herein, are metrics related to human characteristics and used as a form of identification and access control. Scale-based biometrics includes biometrics that are obtained using signals collected by the data-procurement circuitry of the scale (e.g., using electrodes and/or force sensors). Example scale-based biometrics include foot length, foot width, weight, voice recognition, facial recognition, a passcode tapped and/or picture drawn with a foot of the user on the FUI/GUI of the user display, among other biometrics. In some specific embodiments, a scale-based biometric includes a toe-print (e.g., similar to a finger print) that is recognized using a toe-print reader on the FUI/GUI of the scale. The toe print can be used as a secure identification of the user. In other embodiments, the scale-based biometric includes a finger print captured using external circuitry in communication with the scale (e.g., a cellphone or tablet having finger print recognition technology).

The scale further includes a communication activation circuit and an output circuit. The communication activation circuit activates communication between the scale and the remote user-physiologic device in response to the identified scale-based biometric and authorization data received from the remote user-physiologic device. The output circuit receives the validated user data and, in response, displays the user's weight on the user display.

The remote user-physiologic device includes processing circuitry and sensor circuitry. The remote user-physiologic device is configured to collect signals indicative of the user's identity, including the authorization data, and cardio-physiological measurements using the sensor circuitry. Further, the remote user-physiologic device outputs the authorization data to the scale.

Various specific embodiments include methods for pairing a scale and a remote user-physiologic device. For example, various method embodiments include transitioning a scale, in response to a user standing on a platform of the scale, from a reduced power-consumption mode of operation to at least one higher power-consumption mode of operation. The scale includes a user display to display data to a user while the user is standing on the scale, a platform for a user to stand on, data-procurement circuitry, processing circuitry, communication activation circuitry, and an output circuit. The data-procurement circuitry includes force sensor circuitry and a plurality of electrodes integrated with the platform. The processing circuitry includes a CPU and a memory circuit with user-corresponding data stored in the memory circuit. The processing circuitry is arranged within the scale and under the platform upon which the user stands and is electrically integrated with the force sensor circuitry and the plurality of electrodes. The method includes engaging the user with electrical signals, using the data-procurement circuitry, and collecting signals indicative of the user's identity and cardio-physiological measurements while the user is standing on the platform.

Data obtained by the data-procurement circuitry is processed, using the processing circuitry, while the user is standing on the platform and cardio-related physiologic data corresponding to the collected signals is generated therefrom. Further, scale-based biometric of the user are identified using the collected signals, and therefrom, user data, including data indicative of the user's identity and the generated cardio-related physiologic data, is validated as concerning a specific user associated with the scale-based biometrics. Using the communication activation circuitry, communication is activated between the scale and a remote user-physiologic device in response to the identified scale-based biometric and receiving authorization data from the remote user-physiologic device. And, the method includes receiving, by the output circuit, the validated user data and, in response to the validated user data, displaying the user's weight on the user display of the scale.

In various specific embodiments, the remote user-physiologic device includes an application to perform a number of functions using data obtained by the scale and data obtained by the remote user-physiologic device. Alternatively and/or in addition, the processing circuitry of the scale is configured to perform a number function using data obtained by the scale and the remote user-physiologic device. Further, the remote user-physiologic device is in communication with other devices and/or circuitry to perform additional functions and/or enabled further medical assessment.

In certain embodiments, aspects as described herein are implemented in accordance with and/or in combination with aspects of the underlying PCT Application (Ser. No. PCT/US2016/062505), entitled “Remote Physiologic Parameter Assessment Methods and Platform Apparatuses”, filed on Nov. 17, 2016, PCT Application (Ser. No. PCT/US2016/062484), entitled “Scale-Based Parameter Acquisition Methods and Apparatuses”, filed on Nov. 17, 2016, Provisional Application (Ser. No. 62/258,238), entitled “Condition or Treatment Assessment Methods and Platform Apparatuses,” filed Nov. 20, 2015, Provisional Application (Ser. No. 62/263,385), entitled “Scale-Based Biometric Authorization of Communication Between Scale and A Remote User-Physiologic Device”, filed Dec. 4, 2015, and Provisional Application (Ser. No. 62/266,523) entitled “Social Grouping Using a User-Specific Scale-Based Enterprise System”, filed Dec. 11, 2015, to which benefit is claimed and which are fully incorporated herein by reference.

The above discussion/summary is not intended to describe each embodiment or every implementation of the present disclosure. The figures and detailed description that follow also exemplify various embodiments.

### BRIEF DESCRIPTION OF THE DRAWINGS

Various example embodiments may be more completely understood in consideration of the following detailed description in connection with the accompanying drawings, in which:

FIG. 1a shows an apparatus consistent with aspects of the present disclosure;

FIG. 1b shows an example of a scale activating communication with a remote user-physiologic device, consistent with aspects of the present disclosure;

FIG. 1c shows an example of a scale wirelessly communicating with a remote user-physiologic device, consistent with aspects of the present disclosure;

FIG. 1d shows current paths through the body for the IPG trigger pulse and Foot IPG, consistent with various aspects of the present disclosure;

FIG. 1e is a flow chart illustrating an example manner in which a user-specific physiologic meter/scale may be programmed to provide features consistent with aspects of the present disclosure;

FIG. 2a shows an example of the insensitivity to foot placement on scale electrodes with multiple excitation and sensing current paths, consistent with various aspects of the present disclosure;

FIGS. 2b-2c show examples of electrode configurations, consistent with various aspects of the disclosure;

FIGS. 3a-3b show example block diagrams depicting circuitry for sensing and measuring the cardiovascular time-varying IPG raw signals and steps to obtain a filtered IPG waveform, consistent with various aspects of the present disclosure;

FIG. 3c depicts an example block diagram of circuitry for operating core circuits and modules, including for example those of FIGS. 3a-3b, used in various specific embodiments of the present disclosure;

FIG. 3d shows an exemplary block diagram depicting the circuitry for interpreting signals received from electrodes.

FIG. 4 shows an example block diagram depicting signal processing steps to obtain fiducial references from the individual Leg IPG “beats,” which are subsequently used to obtain fiducials in the Foot IPG, consistent with various aspects of the present disclosure;

FIG. 5 shows an example flowchart depicting signal processing to segment individual Foot IPG “beats” to produce an averaged IPG waveform of improved SNR, which is subsequently used to determine the fiducial of the averaged Foot IPG, consistent with various aspects of the present disclosure;

FIG. 6a shows examples of the Leg IPG signal with fiducials; the segmented Leg IPG into beats; and the ensemble-averaged Leg IPG beat with fiducials and calculated SNR, for an exemplary high-quality recording, consistent with various aspects of the present disclosure;

FIG. 6b shows examples of the Foot IPG signal with fiducials derived from the Leg IPG fiducials; the segmented Foot IPG into beats; and the ensemble-averaged Foot IPG beat with fiducials and calculated SNR, for an exemplary high-quality recording, consistent with various aspects of the present disclosure;

FIG. 7a shows examples of the Leg IPG signal with fiducials; the segmented Leg IPG into beats; and the ensemble averaged Leg IPG beat with fiducials and calculated SNR, for an exemplary low-quality recording, consistent with various aspects of the present disclosure;

FIG. 7b shows examples of the Foot IPG signal with fiducials derived from the Leg IPG fiducials; the segmented Foot IPG into beats; and the ensemble-averaged Foot IPG beat with fiducials and calculated SNR, for an exemplary low-quality recording, consistent with various aspects of the present disclosure;

FIG. 8 shows an example correlation plot for the reliability in obtaining the low SNR Foot IPG pulse for a 30-second recording, using the first impedance signal as the trigger pulse, from a study including 61 test subjects with various heart rates, consistent with various aspects of the present disclosure;

FIGS. 9a-b show an example configuration to obtain the pulse transit time (PTT), using the first IPG as the triggering pulse for the Foot IPG and ballistocardiogram (BCG), consistent with various aspects of the present disclosure;

FIG. 10 shows nomenclature and relationships of various cardiovascular timings, consistent with various aspects of the present disclosure;

FIG. 11 shows an example graph of PTT correlations for two detection methods (white dots) Foot IPG only, and (black dots) Dual-IPG method, consistent with various aspects of the present disclosure;

FIG. 12 shows an example graph of pulse wave velocity (PWV) obtained from the present disclosure compared to the ages of 61 human test subjects, consistent with various aspects of the present disclosure;

FIG. 13 shows another example of a scale with interleaved foot electrodes to inject and sense current from one foot to another foot, and within one foot, consistent with various aspects of the present disclosure;

FIG. 14a shows another example of a scale with interleaved foot electrodes to inject and sense current from one foot to another foot, and measure Foot IPG signals in both feet, consistent with various aspects of the present disclosure;

FIG. 14b shows another example of a scale with interleaved foot electrodes to inject and sense current from one foot to another foot, and measure Foot IPG signals in both feet, consistent with various aspects of the present disclosure;

FIG. 14c shows another example approach to floating current sources is the use of transformer-coupled current sources, consistent with various aspects of the present disclosure;

FIGS. 15a-d show an example breakdown of a scale with interleaved foot electrodes to inject and sense current from one foot to another foot, and within one foot, consistent with various aspects of the present disclosure;

FIG. 16 shows an example block diagram of circuit-based building blocks, consistent with various aspects of the present disclosure;

FIG. 17 shows an example flow diagram, consistent with various aspects of the present disclosure;

FIG. 18 shows an example scale communicatively coupled to a wireless device, consistent with various aspects of the present disclosure; and

FIGS. 19a-c show example impedance as measured through different parts of the foot based on the foot position, consistent with various aspects of the present disclosure.

While various embodiments discussed herein are amenable to modifications and alternative forms, aspects thereof have been shown by way of example in the drawings and will be described in detail. It should be understood, however, that the intention is not to limit the disclosure to the particular embodiments described. On the contrary, the intention is to cover all modifications, equivalents, and alternatives falling within the scope of the disclosure including aspects defined in the claims. In addition, the term “example” as used throughout this application is only by way of illustration, and not limitation.

### DESCRIPTION

Aspects of the present disclosure are believed to be applicable to a variety of different types of apparatuses, systems, and methods involving activating communication between a scale and a remote user-physiologic device using a scale-based biometric and remote user-physiologic device-based authorization data. In certain implementations, aspects of the present disclosure have been shown to be beneficial when used in the context of a weighing scale with electrodes configured for engaging with the user and generating cardio-related physiologic data, such as data indicative of a BCG or ECG of a user. In some embodiments, in response to the activation, the scale outputs user data to the remote user-physiologic device and/or the remote user-physiologic device outputs cardio-related physiologic data generated by the remote user-physiologic device to the scale. In order to output the data, the scale receives both authorization from the scale, e.g., biometric, and authorization from the remote user-physiologic device, e.g., authorization data, such that health data is only communicated when both devices are authorized. The remote user-physiologic device and/or the scale uses the user data obtained by the scale and various cardio-related physiologic data generated by the remote user-physiologic device to determine additional cardio-health related information. These and other aspects can be implemented to address challenged, including those discussed in the background above. While not necessarily so limited, various aspects may be appreciated through a discussion of examples using such exemplary contexts.

Accordingly, in the following description various specific details are set forth to describe specific examples presented herein. It should be apparent to one skilled in the art, however, that one or more other examples and/or variations of these examples may be practiced without all the specific details given below. In other instances, well known features have not been described in detail so as not to obscure the description of the examples herein. For ease of illustration, the same reference numerals may be used in different diagrams to refer to the same elements or additional instances of the same element. Also, although aspects and features may in some cases be described in individual figures, it will be appreciated that features from one figure or embodiment can be combined with features of another figure or embodiment even though the combination is not explicitly shown or explicitly described as a combination.

Embodiments of the present disclosure are directed to a platform apparatus that provides various features including communicating with other user devices, such as a remote user-physiologic device, in response to a dual authorization of the communication. The dual authorization include verifying a scale-based biometric is associated with a user and verifying authorization data from the other user device is also associated with the user. The platform apparatus, such as a body weight scale, provides various features, such as collecting scale-obtained data including a scale-based biometric and cardio-physiological measurements from a user while the user is standing on the platform apparatus and outputting the scale-obtained data to external circuitry in response to verifying the scale-based biometric in addition to the authorization data received from the external circuitry. The external circuitry additionally provides the feature of collecting signals indicative of the user's identity, including the authorization data, and cardio-physiological data, and outputs the authorization data to the scale. By authorizing communication between the platform apparatus and the external circuitry responsive to a scale-based biometric in addition to authorization data from the external circuitry, user sensitive data such as health data is only communicated when both devices are authorized and/or in response to the platform apparatus verifying both devices are being used by the same user.

In accordance with a number of embodiments, physiological parameter data is collected using an apparatus, such as a weighing scale or other platform device that the user stands on. The user (owners of a scale or persons related to the owner, such as co-workers, friends, roommates, colleagues), may use the apparatus in the home, office, doctors office, or other such venue on a regular and frequent basis, the present disclosure is directed to a substantially-enclosed apparatus, as would be a weighing scale, wherein the apparatus includes a platform which is part of a housing or enclosure and a user-display to output user-specific information for the user while the user is standing on the platform. The platform includes a surface area with electrodes that are integrated and configured and arranged for engaging a user as he or she steps onto the platform. Within the housing is processing circuitry that includes a CPU (e.g., one or more computer processor circuits) and a memory circuit with user-corresponding data stored in the memory circuit. The platform, over which the electrodes are integrated, is integrated and communicatively connected with the processing circuitry. The processing circuitry is programmed with modules as a set of integrated circuitry which is configured and arranged for automatically obtaining a plurality of measurement signals (e.g., signals indicative of cardio-physiological data) from the plurality of electrodes. The processing circuitry generates, from the signals, cardio-related physiologic data manifested as user-data.

The scale, in various embodiments, includes output circuitry that outputs various data to other external circuitry. For example, using the output circuitry, the scale outputs user data to a remote user-physiologic device, such as a smartphone, a smartwatch, a tablet, and/or other circuitry and devices. The remote user-physiologic device also includes sensor circuitry and collects signals from the user indicative of the user's identity and cardio-physiological measurements, but at a different biological point of the user than the scale. For example, a smartphone or smartwatch is located near the user's hand and the scale is located near the user's feet. Thereby, data obtained by the scale and the remote user-physiologic device is correlated and/or combined and used to determine various cardio-related data that is of a higher quality (e.g., more accurate, less noise, more information) and/or more detail than data from one of the respective devices.

The correlated data from both devices, in various embodiments, is processed to determine clinical indication data of the user and other data, such as cardio-physiological data and wellness data. The clinical indication data, in various embodiments, includes information that is regulated by a government agency, such as the Food and Drug Administration (FDA), and/or otherwise requires a prescription from a physician for the user to obtain. The clinical indication data is indicative of physical state of the user, such as a disease, disorder, and/or risk for a disease or disorder. The other data, such as the cardio-physiological data and wellness data, by contrast, includes derived measurements and/or generic health information that may be “non-regulated” by agencies, such as the FDA. To correlate (e.g., combine) the data from the two devices, the devices communicate data between one another and/or are otherwise paired. However, the data includes sensitive information that the user may not want disclosed to other persons and/or may otherwise be concerned about the information being obtained by others. Embodiments in accordance with the present disclosure include enabling communication between the scale and the remote user-physiologic device in response to the scale identifying a scale-based biometric from the user and receiving authorization data corresponding to the user from the remote user-physiologic device.

In various embodiments, in response to the dual-authorization, the scale and/or the user-physiological device communicates cardio-related physiologic data to one another. The scale or the remote user-physiologic device further performs various additional features using the cardio-related physiologic data obtained by the scale and cardio-related physiologic data obtained by the remote user-physiologic device. Furthermore, the remote user-physiologic device or the scale, in some specific embodiments, receives data from another remote user-physiologic device and correlates the data from the three devices. Data obtained by the scale and the remote user-physiologic device is correlated and/or combined and used to determine various cardio-related data that is of a higher quality (e.g., more accurate, less noise, more information) and/or more detail than data from one of the respective devices (e.g., combine accelerometer signals from cellphone in hand and from scale).

In various specific embodiments, the authorization of both devices includes biometrics of the user. For example, the scale-based biometric includes foot length, foot width, foot shape, toe print, weight, voice recognition, facial recognition, and a combination thereof. In some specific embodiments, a wearable device, such as a ring, wristband, and/or ankle bracelet can be used to positively identify a user, with or without biometrics. The remote user-physiologic device-based biometric includes a finger print, voice recognition, facial recognition, DNA, iris recognition, typing rhythm, and a combination thereof. Alternatively and/or in addition, the authorization data from the remote user-physiologic device includes a password or other passcode, a device ID, and/or a combination of a biometric and a password, passcode, or device ID.

In accordance with various specific embodiments, the remote user-physiologic device and/or the scale correlates the user data from the scale with signals collected by the remote user-physiologic device. For example, the signals collected by the scale and the signals collected by the remote user-physiologic device are collected at the same time, similar times and/or different times. The correlation includes mapping the user data/signals such that the two data sets correlate to one another. For example, in some specific embodiments, the cardio-physiologic measurements output as user data by the scale includes data indicative of a BCG of the user and the cardio-physiologic measurements generated by the remote user-physiologic device includes data indicative of an ECG of the user. The correlation can include correcting the data to get true phase change between the BCG and ECG. In other embodiments, the scale can collect an ECG from a different location than an ECG collected by the remote user-physiologic device. The correlation includes placing the ECG data from the scale in phase with the ECG data from the remote user-physiologic device, such that the two cardiogram waveforms correspond to one another. In other embodiments, the data includes time stamps and the correlation includes mapping the two data sets based on the time stamps. In various embodiments, the correlated user data and collected signals are stored within a user profile corresponding to the user. The user profile is stored on the memory circuit of the remote user-physiologic device, the scale, and/or is stored on external circuitry, such as using a cloud system.

In a number of specific embodiment, the remote user-physiologic device, scale and/or other external circuitry provides clinical indications by processing the data from the scale and the remote user-physiologic device. The clinical indication includes physiologic parameters, diagnosis, conditions, and/or treatments such as PWV, cardiac output, pre-ejection period and stroke volume, among other data. The clinical indications, in various embodiments, are stored in the user profile corresponding with the user. The remote user-physiologic device, scale, and/or other external circuitry controls access to the user profile by allowing access to clinical indications and other data to a physician and not allowing access to the clinical indications to the users. In various embodiments, the remote user-physiologic device and/or other external circuitry allows access to other data to the user, without a prescription. For example, the remote user-physiologic device, scale, and/or other external circuitry allows access by granting access to the respective profile or portions of the data in the profile and/or by sending the respective data to the scale (or another user device) for display or displaying the data on a user display of the remote user-physiologic device. Example data that is non-regulated by an agency and is provided to the user without a prescription includes bodyweight, body mass index, heart rate, body-fat percentage, and cardiovascular age. By controlling access to the clinical indications, that includes Rx health information, the scale and remote user-physiologic device provides the advanced functions of determining the clinical indications while being sold over-the-counter and the user can access this data through their physician. The clinical indications can be used by the physician for further analysis and/or to provide health advice and/or diagnosis, such as medications. Such information, in some embodiments, is provided as a service and can be used to remotely provide health services.

In accordance with various embodiments, the user data is based on sensing, detection, and quantification of at least two simultaneously acquired impedance-based signals. The simultaneously acquired impedance-based signals are associated with quasi-periodic electro-mechanical cardiovascular functions, and simultaneous cardiovascular signals measured by the impedance sensors, due to the beating of an individual's heart, where the measured signals are used to determine at least one cardiovascular related characteristic of the user for determining the heart activity, health, or abnormality associated with the user's cardiovascular system. The sensors can be embedded in a user platform, such as a weighing scale-based platform, where the user stands stationary on the platform, with the user's feet in contact with the platform, where the impedance measurements are obtained where the user is standing with bare feet.

In certain embodiments, the plurality of impedance-measurement signals includes at least two impedance-measurement signals between the one foot and the other location. Further, in certain embodiments, a signal is obtained, based on the timing reference, which is indicative of synchronous information and that corresponds to information in a BCG. Additionally, the methods can include conveying modulated current between selected ones of the electrodes. The plurality of impedance-measurement signals may, for example, be carried out in response to current conveyed between selected ones of the electrodes. Additionally, the methods, consistent with various aspects of the present disclosure, include a step of providing an IPG measurement within the one foot. Additionally, in certain embodiments, the two electrodes contacting one foot of the user are configured in an inter-digitated pattern of positions over a base unit that contains circuitry communicatively coupled to the inter-digitated pattern. The circuitry uses the inter-digitated pattern of positions for the step of determining a plurality of pulse characteristic signals based on the plurality of impedance-measurement signals, and for providing an IPG measurement within the one foot. As discussed further herein, and further described in U.S. patent application Ser. No. 14/338,266 filed on Oct. 7, 2015, which is herein fully incorporated by reference for its specific teaching of inter-digitated pattern and general teaching of sensor circuitry, the circuitry can obtain the physiological data in a number of manners.

In medical (and security) applications, for example, the impedance measurements obtained from the plurality of integrated electrodes can then be used to provide various cardio-related information that is user-specific including, as non-limiting examples, synchronous information obtained from the user and that corresponds to information in a ballistocardiogram (BCG) and an impedance plethysmography (IPG) measurements. By ensuring that the user, for whom such data was obtained, matches other bio-metric data as obtained concurrently for the same user, medical (and security) personnel can then assess, diagnose and/or identify with high degrees of confidence and accuracy.

In a number of a specific embodiments, the user stands on the scale. The scale, responsive to the user, transitions from a low-power mode of operation to a higher-power mode of operation. The scale may attempt to establish communication with another remote user-physiologic device. However, the communication is not activated until authorization data is obtained by the scale from the user and from the remote user-physiologic device. For example, the scale collects signals using the data-procurement circuitry. From the collected signals, the scale identifies a scale-based biometric corresponding with the user and validates the various user data generated as corresponding to the specific user and associated with a user profile. The remote user-physiologic device, at the same time, before or after, collects signals from the user. For example, while the user is standing on the platform, the user turns their cellphone from a sleep mode to on, and in the process provides a password or a biometric, such as a finger print to the cellphone. Subsequently or prior to the cellphone entering a sleep mode, the user accesses an application that is configured to generate cardio-physiologic measurements using collected signals from the user. The application, upon activation, directs the cellphone to output the password or biometric to the scale or the scale outputs a signal to the cellphone requesting the password or biometric. Alternatively, other authorization data is collected by the remote user-physiologic device in response to the user accessing the application, and, in response, the authorization data is sent to the scale. In response to the scale receiving both the scale-based biometric and the authorization data from the remote user-physiologic device, the scale activates communication between the device. In some embodiments, the signals collected by the scale and by the remote user-physiologic device that are indicative of cardio-physiological measurements is collected in response to the activation of communication. For instance, the collection can be synchronized such that the resulting data corresponds to a similar period of time.

In various embodiments, the remote user-physiologic device collects signals using electrodes that are integrated with and/or within the remote user-physiologic device, such as electrodes added as a cover to the cellphone and that are in communication with the cellphone. The remote user-physiologic device, using the collected signals, generates cardio-physiologic measurements. The data obtained by the scale and the remote-user physiologic device is correlated and/or combined to provide additional information to the user and/or to track progress of the user, among other features.

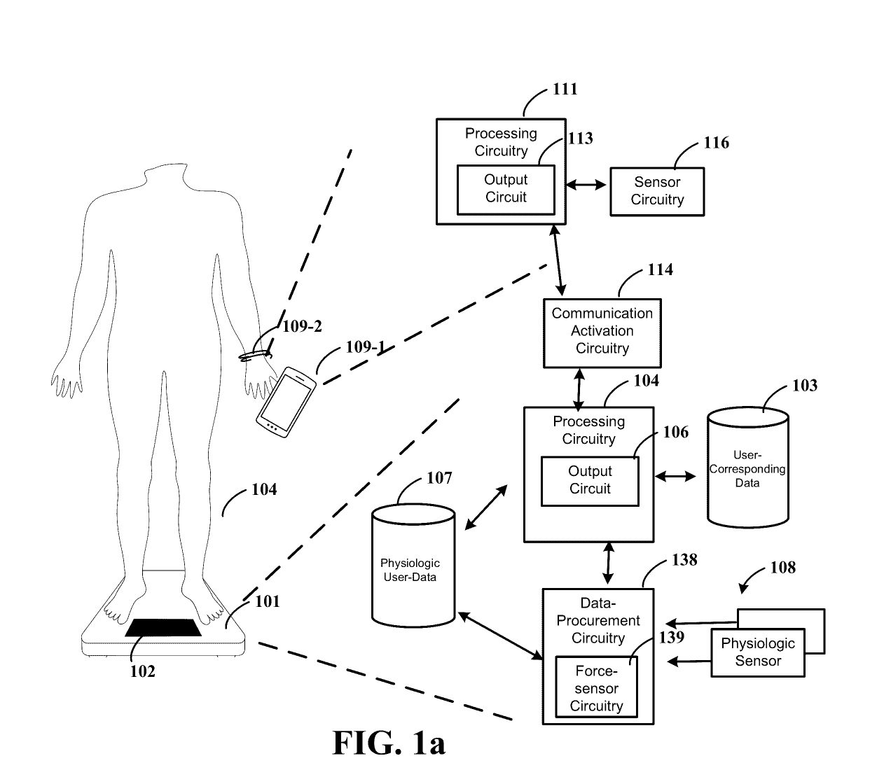

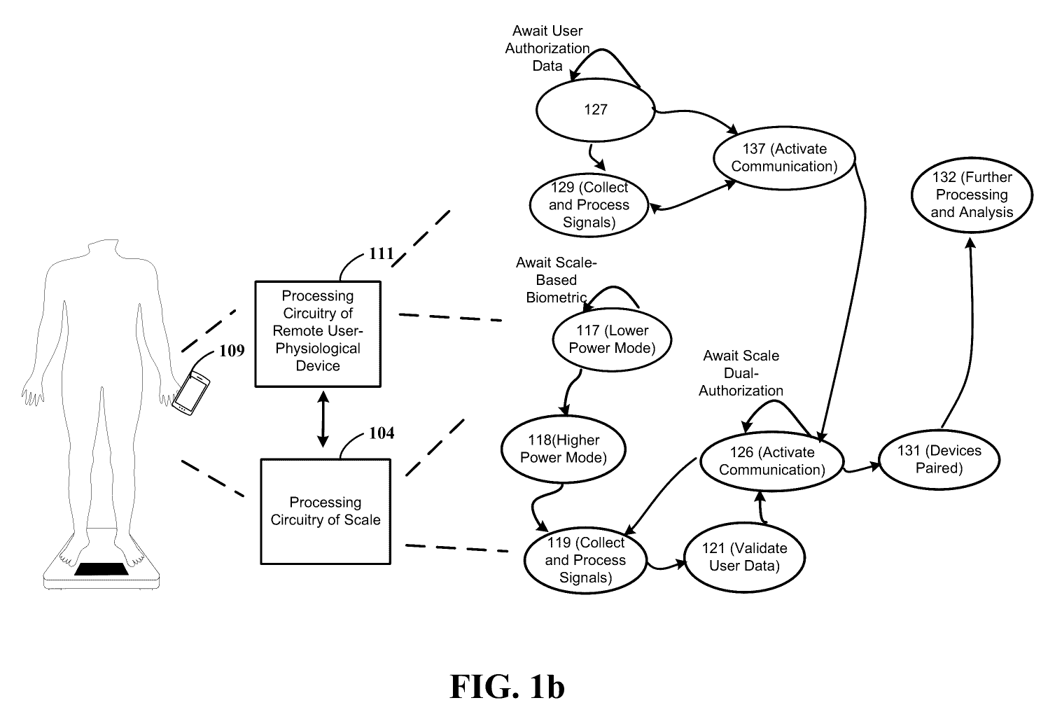

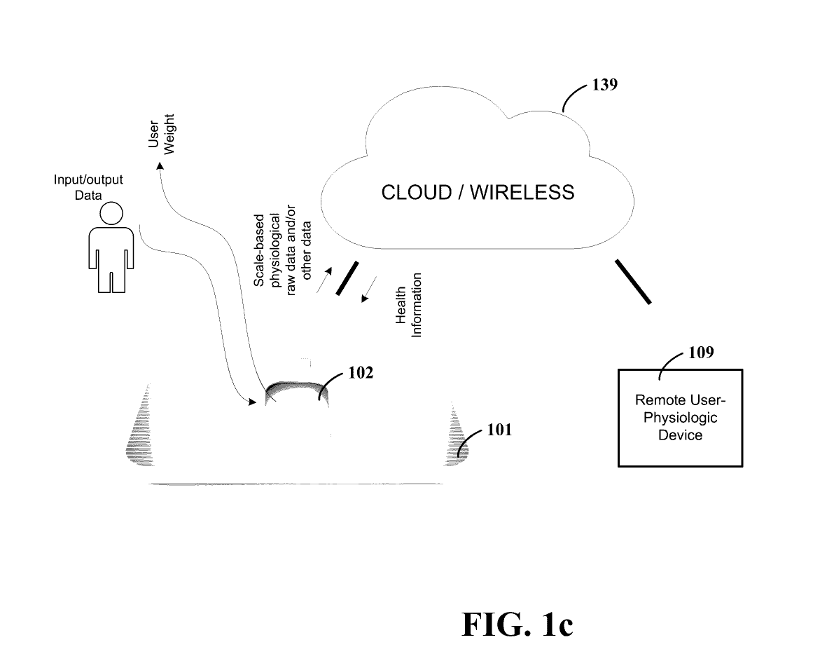

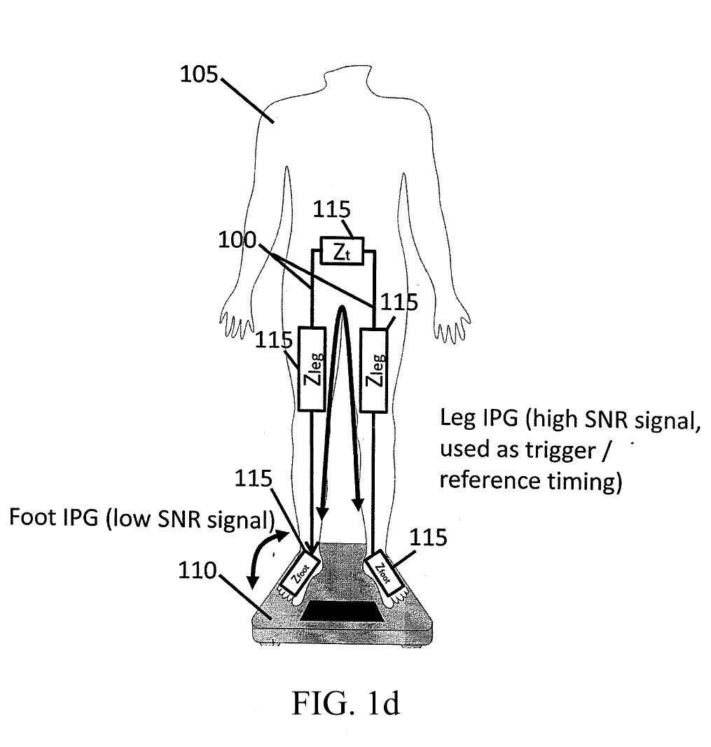

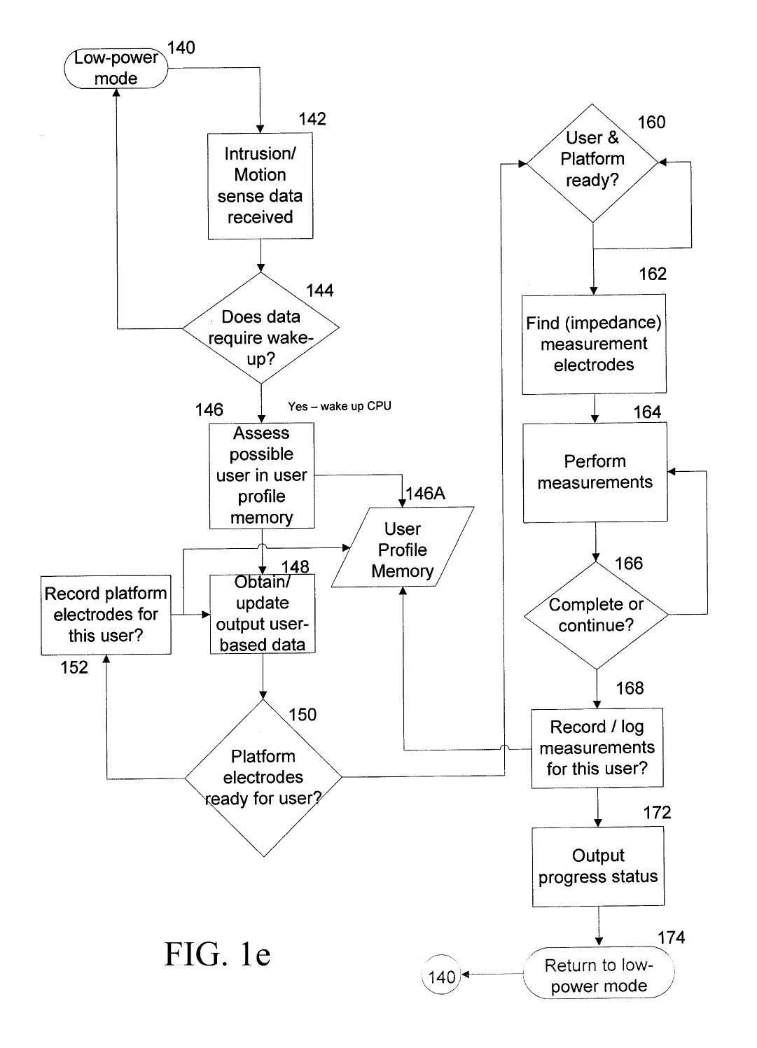

Turning now to the figures, FIG. 1a shows an apparatus consistent with aspects of the present disclosure. The apparatus includes a scale and a remote user-physiologic device (e.g., device 109-1 and/or 109-2). The scale and remote user-physiologic device, in various embodiments, communicate various cardio-related data in response to activation of communication using a dual-authorization. The dual-authorization includes a scale-based biometric and a remote user-physiologic device-based authorization data that both are validated as corresponding to the user. The dual-authorization increases security of sensitive user data and prevent unintended disclosure as compared to a single authorization.

The scale, in various embodiments, includes a platform 101 and a user display 102. The user, as illustrated by FIG. 1a is standing on the platform 101 of the apparatus. The user display 102 is arranged with the platform 101. As illustrated by the dashed-lines of FIG. 1a, the apparatus further includes processing circuitry 104, data-procurement circuitry 138, physiologic sensors 108, communication activation circuitry 114, and an output circuit 106. That is, the dashed-lines illustrate a closer view of components of the apparatus.

The physiologic sensors 108, in various embodiments, include a plurality of electrodes integrated with the platform 101. The electrodes and corresponding force-sensor circuitry 139 are configured to engage the user with electrical signals and to collect signals indicative of the user's identity and cardio-physiological measurements while the user is standing on the platform 101. For example, the signals are indicative of physiological parameters of the user and/or are indicative of or include physiologic data, such as data indicative of a BCG or ECG and/or actual body weight or heart rate data, among other data. Although the embodiment of FIG. 1a illustrates the force sensor circuitry 139 as separate from the physiological sensors 108, one of skill in the art may appreciate that the force sensor circuitry 139 are physiological sensors. The user display 102 is arranged with the platform 101 and the electrodes to output user-specific information for the user while the user is standing on the platform 101. The processing circuitry 104 includes CPU and a memory circuit with user-corresponding data 103 stored in the memory circuit. The processing circuitry 104 is arranged under the platform 101 upon which the user stands, and is electrically integrated with the force-sensor circuitry 139 and the plurality of electrodes (e.g., the physiologic sensors 108).

The data indicative of the identity of the user includes, in various embodiments, user-corresponding data, biometric data obtained using the electrodes and/or force sensor circuitry, voice recognition data, images of the user, input from a user's device, and/or a combination thereof and as discussed in further detail herein. The user-corresponding data includes information about the user (that is or is not obtained using the physiologic sensors 108,) such as demographic information or historical information. Example user-corresponding data includes height, gender, age, ethnicity, exercise habits, eating habits, cholesterol levels, previous health conditions or treatments, family medical history, and/or a historical record of variations in one or more of the listed data. The user-corresponding data is obtained directly from the user (e.g., the user inputs to the scale) and/or from another circuit (e.g., a smart device, such a cellular telephone, smart watch and/or fitness device, cloud system, etc.). The user-corresponding data 103 is input and/or received prior to the user standing on the scale and/or in response to.

In various embodiments, the processing circuitry 104 is electrically integrated with the force-sensor circuitry 139 and the plurality of electrodes and configured to process data obtained by the data-procurement circuitry 138 while the user is standing on the platform 101. The processing circuitry 104, for example, generates cardio-related physiologic data 107 corresponding to the collected signals and that is manifested as user data. Further, the processing circuitry 104 generates data indicative of the identity of the user, such as a scale-based biometric, a user ID and/or other user identification metadata. The user ID is identified, for example, in response to confirming identification of the user using the collected signals indicative of the user's identity (e.g., a scale-based biometric).

The user data, in some embodiments, includes the raw signals, bodyweight, body mass index, heart rate, body-fat percentage, cardiovascular age, balance, tremors, among other non-regulated physiologic data. The user data collected by the scale can further includes force signals, PWV, weight, heartrate, BCG, balance, tremors, respiration, data indicative of one or more of the proceeding data, and/or a combination thereof. In some embodiments, the user data includes the raw force signals and additional physiologic parameter data is determined using external circuitry. Alternatively, the user data can include physiologic parameters such as the PWV, BCG, IPG, ECG that are determined using signals from the data-procurement circuitry and the external circuitry (or the processing circuitry 104 of the scale) can determine additional physiologic parameters (such as determining the PWV using the BCG) and/or assess the user for a condition or treatment using the physiologic parameter. In various embodiments, the processing circuitry 104, with the user display 102, displays at least a portion of the user data to the user. For example, user data that is not-regulated is displayed to the user, such as user weight. Alternatively and/or in addition, the user data is stored. For example, the user data is stored on the memory circuit of the processing circuitry (e.g., such as the physiological user data database 107 illustrated by FIG. 1a). The processing circuitry 104, in various embodiments, correlates the collected user data (e.g., physiologic user data) with user-corresponding data, such as storing identification metadata that identifies the user with the respective data. An algorithm to determine the physiologic data from raw signals can be located on the scale, on another device (e.g., external circuitry, cellphone), and on a Cloud system. For example, the Cloud system can learn to optimize the determination and program the scale to subsequently perform the determination locally. The Cloud system can perform the optimization and programming for each user of the scale.

In some embodiments, the scale collects physiologic data from other devices, such as medical devices, user devices, wearable devices, and/or remote-physiological devices. The data can include glucose measurements, blood pressure, ECG or other cardio-related data, body temperature, among other physiologic data. Further, the scale can act as a hub to collect data from a variety of sources. The sources includes the above-noted user devices. The scale can incorporate a web server (URL) that allows secure, remote access to the collected data. For example, the secure access can be used to provide further analysis and/or services to the user.

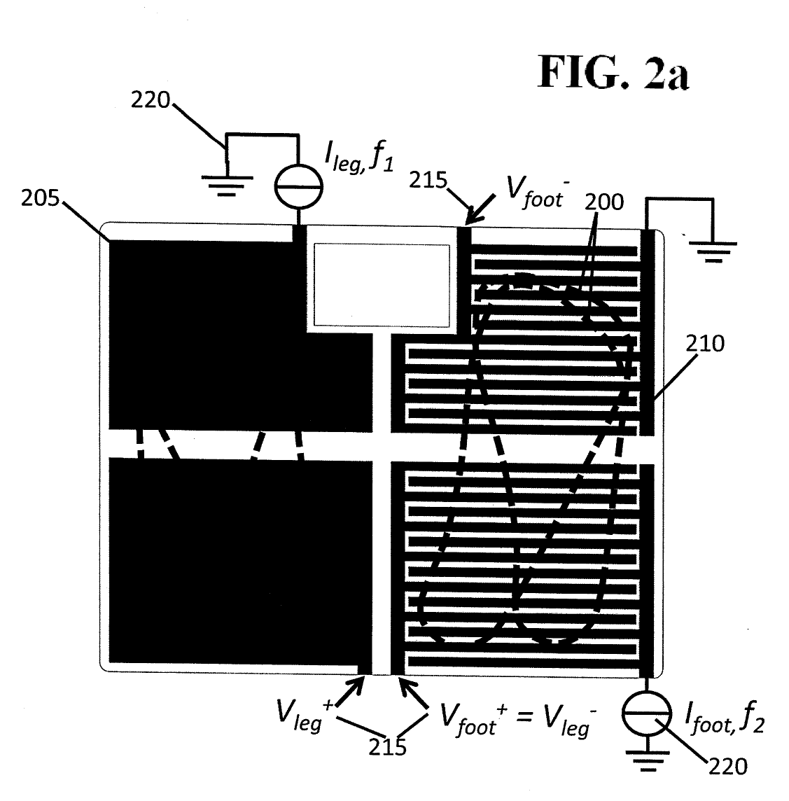

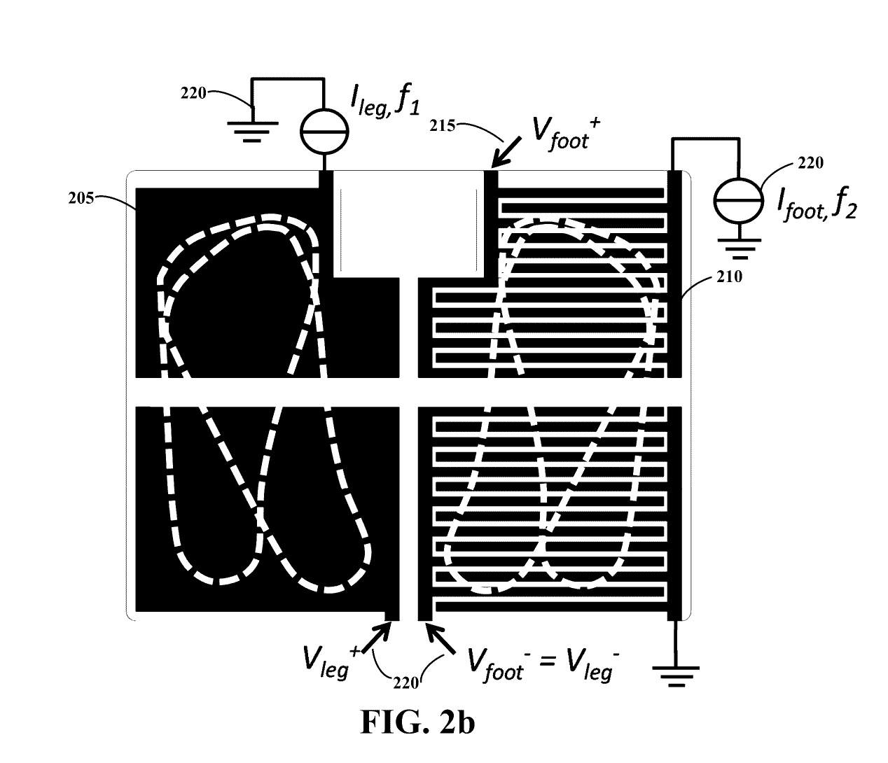

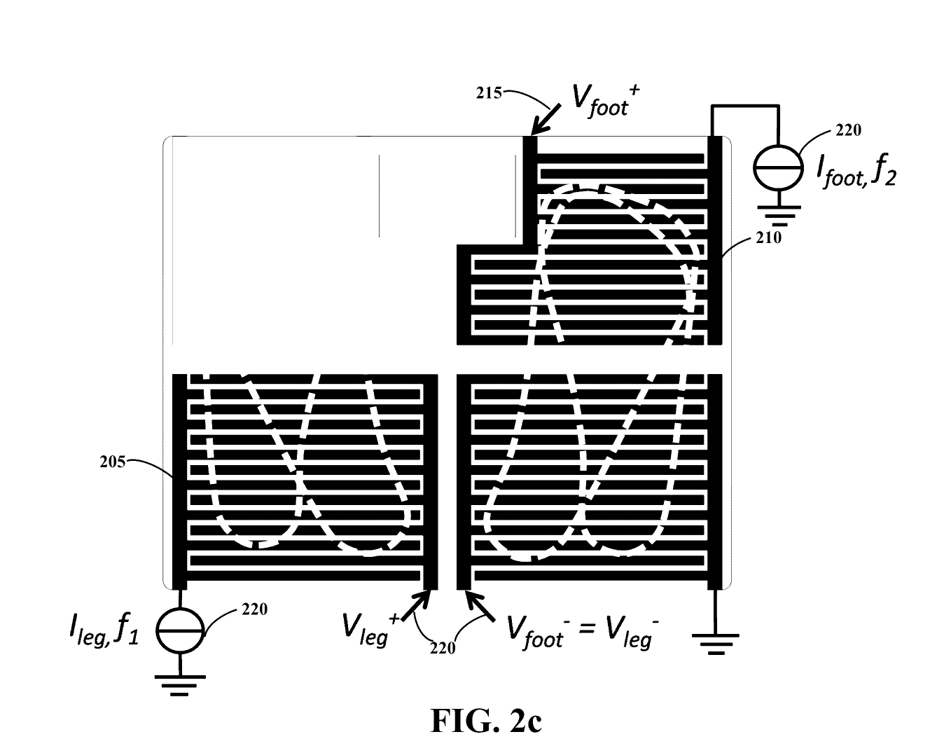

In various embodiment, in response to the user standing on the platform 101, the processing circuitry 104 transitions the scale from a reduced power-consumption mode of operation to at least one higher power-consumption mode of operation. As discussed further herein with regard to FIG. 2a, the different modes of operation, in some embodiments, include a sleep mode that uses a reduced amount of power and an awake mode that uses an additional amount of power as compared to the sleep mode. In a number of embodiments, the user display 102, data-procurement circuitry 138, and the processing circuitry 104 (among other components) transition from the reduced power-consumption mode of operation to the higher power-consumption mode of operation.

The processing circuitry 104 identifies a scale-based biometric of the user using the collected signals. For example, the scale-based biometric includes foot length, foot width, other foot shape, toe print, toe-tapped password, weight, voice recognition, facial recognition, and a combination thereof. In various embodiments, the scale-based biometric corresponds to a user ID and is used to verify identity of the user. Using the scale-based biometric, the user data is validated as concerning the user associated with the scale-based biometric. The user data includes data indicative of the user's identity and the generated cardio-related physiologic data.

The remote user-physiologic device, e.g., device 109-1 and/or 109-2, as illustrated, is not integrated within the scale and, in various embodiments, includes a cellphone, a smartwatch, other smart devices, a tablet, a (photo) plethysmogram a two terminal ECG sensor, and a combination thereof. The remote user-physiologic device includes sensor circuitry 116, processing circuitry 111, and an output circuit 113. The remote user-physiologic device is configured to collect various signals. For example, the remote user-physiologic device collects signals indicative of the user's identity. The collected signals indicative of the user's identity include the authorization data to be sent to the scale to authorize communication. For example, the remote user-physiologic device identifies the authorization data of the user using the collected signals indicative of the user's identity and, therefrom, validates the collected signals as concerning the user associated with the authorization data and/or a user profile.

Example authorization data includes data selected from the group consisting of a password, a passcode, a biometric, a cellphone ID, and a combination thereof. A remote user-physiologic device-based biometric, in various embodiments, includes biometrics selected from the group consisting of: a finger print, voice recognition, facial recognition, DNA, iris recognition, typing rhythm, and a combination thereof, in various embodiments. Responsive to collecting the authorization data and/or verifying the authorization data as corresponding to the user, the remote user-physiologic device outputs the authorization data to the scale. The authorization data is collected, in various embodiments, prior to, during, and/or after, the scale collects various signals.

The scale receives the authorization data and, in response to both the authorization data and the scale-based biometric corresponding to the user, activates communication between the scale and the remote user-physiologic device. For example, the communication activation circuitry 114 activates the communication. The communication activation circuitry 114, in some embodiments, includes an AND gate to active the communication in response to receiving both the identified scale-based biometric and the authorization data that correspond to the same user. Although embodiments are not so limited and the communication activation circuitry can include various circuit components and/or processing circuitry to activate the communication and/or verify both the scale-based biometric and the authorization data correspond to the specific user. Further, in various specific embodiments, the activation enables pairing between the scale and the remote user-physiologic device that includes bi-directional communication.

In response to the activation, an output circuit 106 sends user data to the remote user-physiologic device. For example, the output circuit 106 receives the user data from the processing circuitry 104 and, in response to the user data and the activation of the communication, sends the user data to the remote user-physiologic device. In various embodiments, the output circuit 106 provides data to user via a user interface. The user interface can be integrated with the platform 101 (e.g., internal to the scale) and/or can be integrated with external circuitry that is not located under the platform 101. In some embodiments, the user interface is a plurality of user interfaces, in which at least one user interface is integrated with the platform 101 and at least one user interface is not integrated with the platform 101.

A user interface includes or refers to interactive components of a device (e.g., the scale) and circuitry configured to allow interaction of a user with the scale (e.g., hardware input/output components, such as a screen, speaker components, keyboard, touchscreen, etc., and circuitry to process the inputs). A user display includes an output surface (e.g., screen) that shows text and/or graphical images as an output from a device to a user (e.g., cathode ray tube, liquid crystal display, light-emitting diode, organic light-emitting diode, gas plasma, touch screens, etc.) For example, output circuit can provide data for display on the user display 102 the user's weight and the data indicative of the user's identity and/or the generated cardio-related physiologic data corresponding the collected signals. Alternatively and/or in addition, the remote user-physiologic device, including an output circuit 113, sends signals indicative of cardio-physiologic data to the scale. The communication, in various embodiments, includes a wireless communication and/or utilizes a cloud system.

The user interface is or includes a graphical user interface (GUI), a foot-controlled user interface (FUI), and/or voice input/output circuitry. The user interface can be integrated with the platform 101 (e.g., internal to the scale) and/or can be integrated with external circuitry that is not located under the platform 101. In some embodiments, the user interface is a plurality of user interfaces, in which at least one user interface is integrated with the platform 101 and at least one user interface is not integrated with the platform 101. Example user interfaces include input/output devices, such as display screens, touch screens, microphones, etc.

A FUI is a user interface that allows for the user to interact with the scale via inputs using their foot and/or via graphic icons or visual indicators near the user's foot while standing on the platform. In specific aspects, the FUI receives inputs from the user's foot (e.g., via the platform) to allow the user to interact with the scale. The user interaction includes the user moving their foot relative to the FUI, the user contacting a specific portion of the user display, etc.

A GUI is a user interface that allows the user to interact with the scale through graphical icons and visual indicators. As an example, the external circuitry includes a GUI, processing circuitry, and output circuitry to communicate with the processing circuitry of the scale. The communication can include a wireless or wired communication. Example external circuitry can include a wired or wireless tablet, a cellphone (e.g., with an application), a smartwatch or fitness band, smartglasses, a laptop computer, among other devices. In other examples, the scale includes a GUI and voice input/output circuitry (as further described below) integrated in the platform 101. The user interact with the scale via graphical icons and visual indicators provided via the GUI and voice commands from the user to the scale.

Voice input/output circuitry (also sometimes referred to as speech input/output) can include a speaker, a microphone, processing circuitry, and other optional circuitry. The speaker outputs computer-generated speech (e.g., synthetic speech, instructions, messages) and/or other sounds (e.g., alerts, noise, recordings, etc.) The computer-generated speech can be predetermined, such as recorded messages, and/or can be based on a text-to-speech synthesis that generates speech from computer data. The microphone captures audio, such a voice commands from the user and produces a computer-readable signal from the audio. For example, the voice input/output circuitry can include an analog-to-digital converter (ADC) that translates the analog waves captured by the microphone (from voice sounds) to digital data. The digital data can be filtered using filter circuitry to remove unwanted noise and/or normalize the captured audio. The processing circuitry (which can include or be a component of the processing circuitry 104) translates the digital data to computer commands using various speech recognition techniques (e.g., pattern matching, pattern and feature matching, language modeling and statistical analysis, and artificial neural networks, among other techniques).

The remote user-physiologic device and/or the scale receives the user data and validates the user data as concerning a specific user associated with a user profile (based on the communication activation and/or a user ID within the user data). The remote user-physiologic device, using the sensor circuitry 116 and the processing circuitry 111, collects signals indicative of cardio-physiological data. For example, the sensor circuitry 116, includes electrodes and/or other circuitry configured and arranged to collect the signals. The signals include recordings of electrical activity of the user's heart over a period of time and that are collected by placing electrodes on the user's body. The electrodes detect electrical changes on the skin and/or other surface that arise from the heart muscle depolarizing during each heartbeat. That is, the signals are indicative, in various embodiments, of an ECG of the user. The processing circuitry 111 of the remote user-physiologic device receives the collected signals, and, therefrom generates the cardio-physiological data (e.g., the ECG). Thereby, the remote user-physiologic device includes a two-terminal ECG sensor and/or a plethysmogram sensor, in various embodiments. In a number of specific embodiments, as further discussed herein, the scale and remote user-physiologic device time synchronize prior to obtaining the data.

In various embodiments, the remote user-physiologic device and/or the scale correlates the cardio-physiologic data obtained by the scale with the cardio-physiologic data obtained by the remote user-physiologic device. The correlation includes placing the data in phase, in the same and/or similar time range, in the same and/or similar time scale, and/or other correlation. For example, the cardio-physiologic data from the scale, in a number of embodiments, includes data indicative of a BCG and the cardio-physiologic data from the remote user-physiologic device includes data indicative of an ECG. The correlation can include correcting the data to get true phase change between the BCG and ECG. In other embodiments, the scale can collect an ECG from a different location than an ECG collected by the remote user-physiologic device. The correlation includes placing the ECG data from the scale in phase with the ECG data from the remote user-physiologic device, such that the two cardiogram waveforms correspond to one another. Alternatively and/or in addition, the BCG and ECG data includes time stamps and the correlation includes matching the data based on the time stamps. The correlated data is stored in a user profile corresponding with the user, such as a user profile stored on the remote user-physiologic device, scale, and/or an external circuitry.

In accordance with various embodiments, a communication is activated and/or enabled in response to a dual-authorization, one from the scale and the other from the remote user-physiologic device. In many instances, the scale (or the remote user-physiologic device) are used by multiple people. For instance, the scale may be located in a home, a working environment, a fitness center, a physician office, among other locations. When the scale is located at a public locations, many people may use the scale and users' may not want their cardio-related data and/or weight information to be output to other users. The scale outputs specific user data to a remote user-physiologic device in response to the authorization from both the scale and the remote user-physiologic device that corresponds to the specific user. In other instances, the scale may be located at private location and may track user data for one or more persons living in the private location. The scale outputs specific user data, similarly to the private location as previously discussed, in response to the dual-authorization. Further, in some instances, the scale may correspond to only one user. However, other people visiting the user may stand on the scale as a scale is a common house hold item. Data is communicated to the remote user-physiologic device (which a visiting person may have or be using) or the scale in response to the dual-authorization. The dual-authorization thereby prevents user data corresponding to the user from being communicated to nearby remote user-physiologic devices that the particular user is not using and/or to a remote user-physiologic device when the particular user is not standing on the scale.

In various embodiments, the scale is used by multiple different users. One or more of the different users can have different verifications and different levels of communication modes to display data on the scale, on the remote physiologic device, on another user device, and/or to allow for communication between the scale and the remote physiologic device. For example, a first user may not have other devices and/or prefers to view data while standing on the scale. The first user may be older than a second user. The second user has a remote physiologic device and often views data on the GUI of the remote physiologic device (or another user device, such as a cellphone). A third user may be older than both the first and second user, and may have multiple user devices and one or more remote physiologic devices and may have an identified health concern. When the first user stands on the scale, the scale recognizes the first user via a first biometric and displays data to the first user via the scale and at font and/or size that is larger than when the second user stands on the scale. When the second user stands on the scale, the scale recognizes the second user via a first biometric and authorizes communication between the scale and a remote physiologic device. Further, after correlating the data sets from the two devices, the scale displays some data (e.g., default data such as weight) and an indication of that other data can be viewed on the user device and/or the remote physiologic device, which is displayed on the user interface of the scale, and the second user can view the other data via the GUI of the user device and/or the remote physiologic device responsive to the scale recognizing a second biometric that is more specific or high-level than the first biometric. Similarly, when the third user stands on the scale, the scale recognizes the third user via a first biometric and displays default data and/or an indication that additional data is available via the scale (and at a size and/or font that is larger than what is displayed for the first users). The scale activates communication between the scale and the remote physiologic device of the third user and correlates the data. Further, the scale outputs at least a portion of the user data to the user device (or the remote physiologic device) of the third user responsive to recognizing a second biometric of higher level than the first biometric and outputs user data to external circuitry of a professional for diagnosis and/or other purposes responsive to recognizing a third biometric of a higher level than the second biometric.

The scale can be used in different settings and can have different display defaults depending on the different settings. The different settings can include a consumer setting, a professional setting, and/or a combination. A consumer setting includes or refers to use of the scale in a location of a consumer, such that the multiple users known one another. A professional setting includes or refers to use of the scale in the location of a professional and/or a business, such as a medical office, an exercise facility, a nursing home, etc. In a professional setting, the different users may not know one another and/or know each other less closely than in a consumer setting. A combination setting includes or refers to use of the scale in a location of the consumer with data being output to a professional and/or use of the scale in a location of a professional or business with data output to the user.

Data provided to the user and/or the professional can default to be displayed on a user interface of the scale, the GUI of the user device (such as the remote physiologic device), and/or a GUI of other external circuitry depending on the use of the scale. Depending on the setting, the scale defaults to different default displays. In a consumer setting and/or combination setting, data can default to display on a user display of the scale. The defaulted display of data can be revised by the user providing inputs to display the data on the GUI of the user device or a GUI of another external circuitry (e.g., a standalone CPU) and/or automatically by the scale based on past scale-based actions of the user. As a specific example, a first user provides a user input to the scale to display data on the GUI of the user device multiple times (e.g., more than a threshold number of times, such as five times). In response, the scale adjusts the defaulted display and outputs data to the GUI of the user device. In a professional setting, the scale is not owned by the user. The user may be uninterested in synchronizing their user device with the professional's scale. The display of data may default to the GUI of the user device to display an option to synchronize. Alternatively, the display of data may default to the user interface of the scale to display an option to synchronize and, responsive to user verification or authority to synchronize, defaults to display on the GUI of the user device. During the combination consumer/professional setting, portions of scale-obtained data for a particular user may default to display on external circuitry, such as a standalone or server CPU that is accessible by the professional. The scale, in various embodiments, is aware of the setting based on inputs to the scale, a default setting, and/or querying users of the scale.

The tiered levels of biometrics used to enable communication to external circuitry (from the scale) can include different levels depending on the setting the scale is used in. In a consumer setting and/or combination setting, a first level biometric (e.g., low level or security) can be used to communicate a first subset of low security data to external circuitry accessible and/or belonging to the particular user, such as weight, heartrate, BMI, etc. A second (or more) level biometric, which is higher or more secure than the first level biometric, can be used to communicate a second subset of data that is of a higher security than first subset of data, such as BCG, PWV, condition assessment, etc. to the external circuitry accessible and/or belonging to the user. A third (or more) level biometric, which is higher or more secure than the second level biometric, can be used to communicate a subset (which may include all or a portion of the first and second subset) of data to external circuitry that does not belong to the user (e.g., professional's circuitry, server circuitry, etc.). In a professional setting, a first level (low level or security) biometric can be used to communicate data to external circuitry and/or portals that are associated with the professional. A second level biometric, that is a higher level or security than the first level biometric, can be used to communicate to other circuitry that does not belong to the professional, such as server CPU that is accessible by the user and/or a third party. Although embodiments are not limited to the above provided example, which are provided for illustrative purposes. For example, more or less levels of biometrics can be used in various embodiments.

In various embodiments, the correlated user data and data from the remote user-physiologic device is further processed and/or analyzed. For example, using the correlated data, the remote user-physiologic device, scale, and/or other external circuitry medically assess the user, provides clinical indications, provides generic health information that correlates to the correlated data, and controls access to the various data, among other analysis. For example, using the cardio-physiological data from the scale and the remote user-physiologic device, the remote user-physiologic device and/or scale determine cardio-related data. The cardio-related data includes physiological parameters, such as a cardiac output, a PWV, a revised BCG or ECG, pre-ejection period, stroke volume, arterial stiffness, respiration, and/or other parameters. Further, using the cardio-related data, the remote user-physiologic device and/or scale derives clinical indication data. The clinical indication data, as used herein, is indicative of a physiological status of the user and can be used for assessment of a condition or treatment of the user. Example clinical indication data includes physiological parameters, risk factors, and/or other indicators that the user has a condition or could use a treatment. For example, the user is correlated with the condition or treatment by comparing the cardio-related data to reference information. The reference information, in various embodiments, includes a range of values of the cardio-related data for other users having the corresponding condition or treatment indicators. The other users are of a demographic background of the user, such that the reference information includes statistical data of a sample census.

For example, in specific embodiments, in response to the user standing on the scale, the scale transitions from the reduced-power mode of operation to the higher-power mode of operations and collects signals indicative of user's identity. In response to the transition, the scale collects signals indicative of cardio-physiological measurements (e.g., force signals). The processing circuitry 104 identifies a scale-based biometric using the collected signals and processes the signals to generate cardio-related physiologic data manifested as user data. Further, the processing circuitry validates user data, which includes data indicative of the user's identity and the cardio-related physiologic data, as concerning the user associated with the scale-based biometric. Optionally, the validation includes correlating the user data with a user ID in response to the validation. During, after, and/or before the identification of the scale-based biometric, the remote user-physiologic device collects signals indicative of the user's identity and, therefrom, identifies authorization data corresponding to the user. The remote user-physiologic device outputs the authorization data to the scale. In response to the scale identifying the scale-based biometric as corresponding to the user and receiving the authorization data corresponding to the user, the scale activates communication between the devices. For example, the scale outputs the user data to the remote user-physiologic device in response to activation of the communication.

In some embodiments, the scale-based biometric and the authentication data are received at different times. In such embodiments, the communication activation circuitry 114 may activate the communication in response to receiving each of the scale-based biometric and the authentication data within a threshold period of time (e.g., 60 seconds, 5 minutes, 10 minutes). In response to receiving one of the scale-based biometric and the authentication data outside the threshold period of time, the scale may not activate the communication and/or triggers each device to resend the scale-based biometric and the authentication data.

In accordance with a number of embodiments, as discussed further herein, the remote user-physiologic device or the scale provides additional health information to the user using the user data from the scale and the cardio-related physiologic data generated by the remote user-physiologic device. The remote user-physiologic device (and/or the scale), for example, receives user input data that indicates the user is interested in additional (non-Rx) health information and various categories of interest. The categories of interest, in number of embodiments, include demographics of interest, symptoms of interest, disorders of interest, diseases of interest, drugs of interest, treatments of interest, etc. The additional health information is derived by the remote user-physiologic device (or the scale) and displayed to the user using a display of the remote user-physiologic device. The remote user-physiologic device and/or the scale further communicates the additional health information to another circuitry such that the user can print the additional health information.

The scale can be used to determine or obtain the categories of interest. In accordance with a number of embodiments, the scale performs a question and answer session. For example, the FUI can display a plurality of questions using the user display. Using user interaction by the user's foot, the FUI receives user inputs (e.g., answers) to each of the questions and, using the output circuit, stores the user inputs within a user profile associated with the user. For example, the FUI provides a number of questions in a question and answer session to identify symptoms, health or fitness goals, categories of interest, demographic information, and/or other data from the user. As previously described, the scale can (alternatively and/or in addition to a FUI or GUI) have a voice input/output circuitry that can obtain user's answers to questions via voice comments and inputs user information in response (e.g., a speaker component to capture voice sounds from the user and processing circuitry to recognize the voice commands and/or speech). In a specific example, when the user stands on the platform of the scale, and the scale detects touching of the toe, the scale can reject the toe touch (or tap) as a foot signal (e.g., similar to wrist rejection for capacitive tablets with stylus).

Although the present examples embodiments provided above are in reference to remote user-physiologic device performing the determination, embodiments in accordance with the present disclosure are not so limited. For example, the processing circuitry 104 of the scale and/or other external circuitry determine the clinical indication while the user is standing on the platform 101.

In a number of embodiments, the remote user-physiologic device and/or the scale determines additional physiologic parameters and/or data, such as further clinical indications, of the user using the determined physiologic parameter. For example, the determined physiologic parameter include an ECG and the external circuitry 111 can determine a BCG using the ECG. Alternatively and/or in addition, the external circuitry 111 determines a health status of the user using the determined physiologic parameter, such as a condition or treatment.

FIG. 1b shows an example of a scale activating communication with a remote user-physiologic device consistent with aspects of the present disclosure. The apparatus, as illustrated by FIG. 1b includes a scale and a remote user-physiologic device 109. The scale and the remote user-physiologic device 109 illustrated by FIG. 1b is the same scale and the remote user-physiologic device (e.g., devices 109-1 and 109-2) as previously illustrated and discussed with regard to FIG. 1a. Thereby, the scale includes a platform and data-procurement circuitry in which force-sensor circuitry and a plurality of electrodes (e.g., the physiologic sensors 108) are integrated with, processing circuitry 104 to receive signals from the electrodes and, in response, derive user data to the external circuitry 111. The processing circuitry 104 includes a CPU and a memory circuit with user-corresponding data stored in the memory circuit. As previously discussed, the scale includes communication activation circuitry and an output circuit.

In various embodiments, the scale activates communication between the scale and the remote user-physiologic device 109 in response to a scale-based biometric and a remote user-physiologic device-based authentication data corresponding to the same user. For example, the scale waits for a user to stand on the platform. User-corresponding data, in various embodiments, is input and/or received prior to the user standing on the scale and/or in response to. In response to the user standing on the scale, the scale transitions from a reduced power-consumption mode of operation 117 to at least one higher power-consumption mode of operation 118. At block 119, the scale collects signals indicative of an identity of the user and cardio-physiological measurements (e.g., force signals) by engaging the user with electrical signals and, therefrom, collecting the signals. Further, at block 119, the processing circuitry 104, processes the signals obtained by the data-procurement circuitry while the user is standing on the platform and generates, therefrom, cardio-related physiologic data corresponding to the collected signals.

At block 121, the processing circuitry 104 identifies a scale-based biometric of the user using the collected signals and validates the user data, which includes the data indicative of the users identity and the generated cardio-related physiologic data, as concerning the user associated with the scale-based biometric. At block 126, the scale waits for dual-authorization. The dual-authorization includes the communication activation circuit of the scale receiving a scale-based biometric corresponding to a specific user and authorization data from the remote user-physiologic device 109 corresponding to the same specific user.

The remote user-physiologic device 109, as previously discussed, includes a device, including processing circuitry 111, configured to collect various signals from the user. In various embodiments, the remote-physiologic device 109 is configured to operate in multiple modes. For example, the remote user-physiologic device 109, at block 127, waits for user authorization data from the user. The user authorization data, as previously discussed, includes the user entering a password or finger print to the remote user-physiologic device 109 to transition the remote user-physiologic device 109 from a reduced-power mode of operation to a higher-power mode of operation. Alternatively and/or in addition, the user authorization data includes a password, pass code, and/or biometric data obtain in response to the user accessing the specific functionality (e.g., an application) of the remote user-physiologic device 109 capable of generating cardio-related physiologic data.

In response to the authorization data, at block 129, the remote user-physiologic device 109 collects signals indicative of the cardio-physiologic data and generates therefrom the cardio-physiologic data. Further, at block 137, the remote user-physiologic device 109 activates the communication by outputting the authorization data to the scale. The authorization data is output concurrently, during, and/or after the collection of signals indicative of the cardio-physiologic data by the remote user-physiologic device 109.

At block 131, in response to the identified scale-based biometric and receiving the authorization data from the remote user-physiologic device 109 corresponding to the same user, the scale activates the communication between the scale and the remote user-physiologic device 109. As illustrated by FIG. 1b, the activation includes pairing the scale and the remote user-physiologic device 109, in a number of embodiments. Further, the scale, in various embodiments, displays the user's weight on the display of the scale. And, in response to activation, the scale sends the user data from the scale to the remote user-physiologic device 109 and/or the remote user-physiologic device 109 sends signals indicative of cardio-physiologic related data to the scale. At block 132, the remote user-physiologic device 109 and/or the scale, further processes and analyzes the cardio-related physiologic data from the scale and from the remote user-physiologic device 109.

In various embodiments, the remote user-physiologic device 109 correlates and stores the user data and the data obtained by the remote user-physiologic device 109 with a user profile of the user. Further, in some embodiments, as previously discussed, the remote user-physiologic device 109 correlates the cardio-related physiologic data generated by the scale with the cardio-related physiologic data generated by the remote user-physiologic device 109. The remote user-physiologic device 109 uses the correlated data to derive cardio-related data that may be of a higher quality and/or have more information than the data individually.

In a number of embodiments, the remote user-physiologic device 109 and/or the scale provides (e.g., determines) clinical indication data by processing the derived cardio-related data, such as determining a physiologic parameter as discussed in further detail herein. The clinical indication data, in various embodiments, include physiologic parameters (such as PWV, BCG, respiration, arterial stiffness, cardiac output, pre-ejection period, stroke volume), diagnosis, conditions, and risk factors, among other health information. The remote user-physiologic device 109 and/or the scale provides the clinical indication, in some embodiments, by updating the user profile of the user with the received user data and/or the clinical indication data.

In various related embodiments, the remote user-physiologic device 109 and/or the scale determines additional health information and provides the additional health information for display to the user. The additional health information is indicative of the clinical indication data and correlates to the categories of interest provided by the user. The categories of interest are provided at a different time, the same time and/or from the scale. In various embodiments, the additional health information is based on historical user-data. For example, the additional health information (e.g., a table) provided include a correlation to the category of interest and the user data over time.

In some embodiments, the remote user-physiologic device 109 and/or the scale controls access to data within the user profile. In some embodiments, the control of access includes allowing access to the clinical indication data and the user data to a physician corresponding to the user for information. Further, the control includes not allowing access to the clinical indication data to the user. In various embodiments, the user is allowed to access the user data in the profile and the remote user-physiologic device 109 displays portions of the user data and/or other non-regulated data. Additionally, the remote user-physiologic device 109 and/or the scale may not allow access to the profile and/or any data corresponding to the profile to non-qualified personal, such as other users. In various embodiments, the user is allowed access the clinical indication data in response to interpretation by the physician and a prescription from the physician to access the clinical indications. Further, in some embodiments, a demographic model and/or other report is provided to the user in response to the clinical indication data. For example, the user may not be allowed to view the clinical indication data but is provided generic information corresponding to other users with similar clinical indications. The user data can be collected and determined but the user is not allowed access to the features, such as access to the user data or service related to the user data until government clearance is obtained. For example, the scale collects and stores the user data but does not display or otherwise allow the user access to the user data until clearance is obtained for each feature, which retrospectively enables the feature and/or service. Alternatively and/or in addition, the feature and/or service is not provided until a weighted value is received (e.g., payment).

The access is controlled, in various embodiments, using a verification process. For example, in response to verifying identification of the physician and/or the user, access to particular data is provided. The verification is based on a user sign in and password, a password, biometric data, etc., and/or identification of the user using the scale (in which, the relevant data is sent to the scale or another user device in response to the identification).

In various embodiments, the clinical indication data is provided as an additional service. For example, the user can obtain the information and/or have their physician interpret the information for a service fee. The service fee includes a one-time fee for a single interpretation, a monthly or yearly service fee, and/or is a portion of a healthcare insurance fee (e.g., the user can purchase a health care plan that includes the service). In such embodiments, the physician corresponding to the user accesses the clinical indication data and/or other user data in response to verification that the user has enabled the service and verification of the identity of the physician.

The controlled access, for example, allows a physician corresponding with the data to access the clinical indication data for interpretation. For example, the physician can give a prescription to the user to access all information in the user profile. In response to the prescription, the remote user-physiologic device 109 and/or the scale allows the user to access the clinical indication data. Further, the physician can prescribe medicine to the user based on the profile and the remote user-physiologic device 109 and/or the scale provides an indication to the user that a prescription for medicine is ready. The physician may provide instructions or further explanation for the user, which is sent and displayed using the scale and/or another user-device. Such information includes life-style suggestions, explanation for how to use the prescribed medicine and/or why it is prescribed, and/or other advice, such as symptoms that the user should watch for. For instance, the clinical indication data may suggest that the user has a heart condition and/or disorder. The physician may prescribe medicine to the user and/or provide potential symptoms that the user should watch for and/or should go to the physician's office or an emergency room if the symptoms arise. In this manner, the scale and/or remote user-physiologic device 109 controls access to Rx health information is used to remotely monitor health of the user and/or provide physician services.

In accordance with various embodiments, although not illustrated by FIG. 1b, the apparatus includes an additional sensor circuitry that is external to the scale and the remote user-physiologic device 109. The additional sensor circuitry includes a communication circuit and is configured and arranged to engage the user with electrical signals and collect therefrom signals indicative of an ECG of the user. The sensor circuitry, which may include and/or be correlated with processing circuitry configured to derive an ECG from the collected signals. The sensor circuitry communicates the ECG to the remote user-physiologic device 109 and the scale communicates a BCG to the remote user-physiologic device 109. The additional sensor circuitry can be located at a different location of the user than the remote user-physiologic device 109 and the scale (e.g., on the wrist, head, or ankle).

In various embodiments, the apparatus includes additional remote user-physiologic devices and/or other body accessories. For example, the scale receives data from a plurality of remote user-physiologic devices and/or other body accessories. The remote user-physiologic device 109 and/or scale receives data from the plurality of remote user-physiologic devices or other body accessories and calibrate the data from each of the remote user-physiologic devices/body accessories. In this way, the scale is used as a hub for collecting and correlating data corresponding to a user. For example, the data can include fitness data, cardio-related data, user input data (e.g., calorie counts/food intake, drug dosage, treatment, sleep schedule), sleep schedule (e.g., directly input from a smartbed and/or other body accessory), among other data. The scale collects the various data and correlates the data with a user profile corresponding with the user. In various embodiments, the data from one of the remote user-physiologic devices may conflict with data obtained by the scale. In such instances, the data obtained by the scale is used and the data from the remote user-physiologic device is discarded. That is, the data from the scale is the default data as the scale may include greater processing resources and/or obtain higher quality signals than the remote user-physiologic device.

Although the present embodiments illustrates the remote user-physiologic device 109 or the scale performing the various additional processing, embodiments are not so limited. For example, external circuitry can perform the processing and update the user profile, which may be stored on the external circuitry, the remote user-physiologic device 109, or the scale. The user profile can be accessed by the scale, the remote user-physiologic device 109, or the external circuitry, in response to authorization. The authorization can, in some embodiments, include dual-authorization. In response to the authorization, various data is displayed to the user, such as on a user display of the remote user-physiologic device 109 or the scale. The user, in various embodiments, can establish where to display data, based on user preferences stored in the user profile.

In some embodiments, the scale-based biometric and the authentication data are received at different times. In such embodiments, the communication activation circuitry may activate the communication in response to receiving each of the scale-based biometric and the authentication data within a threshold period of time (e.g., 60 seconds, 5 minutes, 10 minutes). In response to receiving one of the scale-based biometric and the authentication data outside the threshold period of time, the scale may not activate the communication and/or triggers each device to resend the scale-based biometric and the authentication data.

The scale can time synchronize with the remote user-physiologic device prior to the scale and remote user-physiologic device (or other user devices) obtaining the user data, in various specific embodiments. When using data from both the scale and another device, time-based (e.g., phase) inaccuracies between user data sets from the other device and the scale can impact assimilation and/or combined use of the two sets of user data. For example, lack of time synchrony can cause issues such as cardiac parameters from each device not coordinating, and/or being inaccurate, and/or not identifying the correct data to output. For example, a user exercises while wearing a remote user-physiologic device (e.g., a wearable device) that monitors one or more physiological parameters, and the remote user-physiologic device outputs the physiological parameters to a scale for further processing. The time (e.g., phase) used by the remote user-physiologic device can cause a resulting physiological parameter (e.g., waveform) to be inaccurate. The scale and the remote user-physiologic device (or other user devices) can be time-synchronized based on the frequency and/or timing (e.g., phase) of signals or waveforms. Time-synchronizing includes or refers to synchronizing two waveforms (e.g., signals from the scale and the user device) based on a frequency and a timing, sometimes referred to as “a phase angle”. In specific embodiment, time-synchronized waveforms have the same frequency and same phase angle with each cycle and/or share repeating sequences of phase angles over consecutive cycles.

The following is a specific example of a remote user-physiologic device or other user devices time-synchronizing with a scale prior to obtaining user data. While the user is standing on the scale, the scale recognizes a nearby remote user-physiologic device (e.g., within a threshold) and prompts the user to pair the remote user-physiologic device and scale. The user authorizes the pairing (e.g., selects an icon on the FUI or otherwise provides an indication of an interest) by providing an indication of interest to the scale (e.g., select an icon, provide a voice command, or perform an action). In specific embodiments, the user device and scale can be time-synchronized by tapping the user device, such as a remote user-physiologic device, a wearable device, cellphone, and/or tablet to the scale. The scale synchronizes via strain gauges of the scale and accelerometer of the user device, as previously described. In other specific embodiments, the scale provides a command to the user device, which is placed on the scale and/or tapped on the scale, the scale detects the vibration frequency and timing (e.g., phase). This can be used to give secure identification and time synchronization, as previously described.

In a number of specific embodiments, the user activates a time-synchronization service/feature of the scale. For example, the user stands on the scale and identifies the user device, such as a remote user-physiologic device, including how to synchronize the two devices, using a user interface (e.g., FUI of the scale, external GUI in communication, etc.) The scale authorizes the communication and/or the synchronization by recognizing the user using a scale-based biometric and based on authorization data from the user device, in some specific embodiments. In response to the synchronization, the scale outputs a message requesting a time value from the user device. The user device, in response to the message, outputs a response message with an indication of the time value. The response message can include the user device vibrating (at a respective frequency and timing). The scale detects the vibration at a frequency and timing, and can determine the vibration frequency and timing. The determined vibration frequency and timing can be used to time-synchronize the scale with the user device based on a time difference. A time difference between the scale and the user device can include a difference in relative time (e.g., phase) according to the scale and relative time (e.g., phase) according to the user device. The scale can time-synchronize by outputting a message to the user device to adjust its timing based on the time difference and/or to match the timing of the scale.

As previously described, the time-synchronization can occur responsive to a user dropping and/or tapping the user device on the scale. The user device may include a built-in accelerometer and the user dropping or tapping the user device on the platform of the scale (with or without standing on the scale) can activate the time-synchrony. In various embodiments, the time-synchrony is activated in response to the user device being within a threshold distance from the scale. In other embodiments, the user is standing on the scale and/or within a threshold distance, and the scale outputs a messaged to the user device to vibrate to trigger the time-synchronization, as previous discussed. Further, via NFC, Bluetooth, and/or wireless communication, the time-synchrony can occur through direct communication between the scale and the user device. In some specific embodiments, the time-synchrony occurs in response to verification that the user device (and/or the scale) has recognized the user within a threshold period of time. The verification can be used to mitigate or prevent accidental synchronization and can be used in combination with a user dropping or tapping the user device on the scale and/or the user device being within a threshold distance from the scale.

In other specific embodiments, the scale time-synchronizes with the user device by docking the user device with the scale and/or via acoustic sounds. For example, the user device may be a remote user-physiologic device that includes a photoplethy configured to obtain a photoplethysmogram. The photoplethy can be time-synchronized by docking (e.g., placing on the platform and/or connecting) the remote user-physiologic device with the scale and using a light source of the scale to flash a pattern to calibrate the photoplethy (e.g., flashing LED lights via one or more LEDs embedded in the platform of the scale). Further, the user device can be acoustically calibrated by outputting sounds from the platform (e.g., “pips” and “chirps”).

The scale can include a mechanical mass that can be triggered by the user device to calibrate the system. In response to a command from the user device, for example, a mechanical input is input to circuitry of the scale using the mechanical mass. The scale can pick apart the mechanical input separately from a cardiac parameter (e.g., BCG) and use the mechanical input to measure a phase latency of the system.

FIG. 1c shows an example of a scale activating communication with a remote user-physiologic device 109 consistent with aspects of the present disclosure. The scale is configured to monitor signals and/or data indicative of physiologic parameters of the user while the user is standing on the platform 101. The remote user-physiologic device 109 further monitors signals and/or data indicative of physiologic parameters of the user. The scale controls communication of data between the scale and the remote user-physiologic device based on a dual-authentication of the user using both the scale and the remote user-physiologic device.

As previously discussed, a scale in various embodiments includes a platform 101, a user display 102, processing circuitry, communication activation circuitry, and an output circuit. The output circuitry sends user data to the remote user-physiologic device 109 for further assessment and correlation with cardio-related physiologic data obtain at a different point of the user's body using the remote user-physiologic device 109. Alternatively and/or in addition, the remote user-physiologic device 109 sends cardio-related physiologic data to the scale for further assessment and correlation with user data. The communication activation circuitry activates the communication between the processing circuitry of the remote user-physiologic device 109 and the processing circuit of the scale. The communication is enabled in response to a scale-based biometric and authorization data from the remote user-physiologic device 109, which can include a remote user-physiologic device-based biometric, and both of which correspond to the same specific user.

The scale and remote user-physiologic device 109 communicate data wirelessly (and/or via the cloud 139) to one another. For example, the remote user-physiologic device 109 outputs authorization data to the scale. In response to the authorization data corresponding to the same user as a scale-based biometric obtain using the scale, the scale outputs scale-based physiological raw data and/or user data (or the remote user physiologic device 109 outputs cardio-related physiologic data). Further, the scale displays a user weight to the user, using the user display of the scale.

The remote user-physiologic device 109 or the scale correlates the user data with data obtained by the remote user-physiologic device 109 and, therefrom, generated cardio-related data. In some embodiments, the remote user-physiologic device 109 or the scale outputs various cardio-related data to an external circuitry. For example, in some embodiments, the external circuitry includes a medical file database and the various cardio-related data is automatically populated in the medical file corresponding to the user and for a physician to review. The external circuitry (and/or the remote user-physiologic device) further analyzes the cardio-related data and determine additional health information, such as non-prescription (Rx) health information to provide to the user.

In some embodiments, the remote user-physiologic device 109, the scale, or the external circuitry controls access to various data, such as the clinical indications, by storing the parameter in a database corresponding with and/or integrated with the remote user-physiologic device 109. Alternatively and/or in addition (such as, in response to determining the user can access the parameter) the remote user-physiologic device 109 outputs various data, such as the clinical indication to the scale for display and/or storage.

In accordance with a number of embodiments, the scale and the remote user-physiologic device 109 provide additional health information to the user. The remote user-physiologic device 109, for example, receives user input data that provides an indication that the user is interested in additional (non-Rx) health information and various categories of interest. The categories of interest include demographics of interest, symptoms of interest, disorders of interest, diseases of interest, drugs of interest, treatments of interest, etc. The additional health information is derived by the remote user-physiologic device 109 or the scale and provided to the user.

For example, in a number of embodiments, the remote user-physiologic device 109 including the processing circuitry provides a number of questions to the user. The questions are provided via a speaker component of the remote user-physiologic device 109 outputting computer-generated natural voice (via a natural language interface), displaying the questions on the user display of the remote user-physiologic device 109, and/or outputting the questions to another user-device. In various embodiments, the questions include asking the user if the user is interested in additional health information and if the user has particular categories of interest. In various embodiments, the categories of interest include a set of demographics, disorders, diseases, and/or symptom that the user is interested, and/or other topics. The additional health information includes a table that corresponds to the categories of interest and/or corresponds to the physiological parameter and/or clinical indications determined without providing any specific values and/or indication related to the physiological parameter, among other data. The user is provided the additional health information by the remote user-physiologic device 109 outputting the information to the scale and/or the remote user-physiologic device 109 displays the information. In various embodiments, the information can be printed by the user to bring to a physician. In various related-aspects, the scale using the processing circuitry 104 generates the additional health information instead of the remote user-physiologic device 109. In various embodiments, the user data is compared against historical user data for the same user and used to analyze if the user's condition/treatment and risk is getting better or worse over time.

The additional health information is generated, in various embodiments, by comparing the categories of interest to the cardio-related physiologic data generated by the scale and by the remote user-physiologic device 109. In various embodiments, the correlation/comparison include comparing statistical data of a sample census pertinent to the categories of interest and at least one physiological parameter determined using the cardio-related physiologic data. The statistical data of a sample census includes data of other users that are correlated to the categories of interest. In such instances, the additional health information includes a comparison of data measured while the user is standing on the platform 101 and data measured using the remote user-physiologic device 109 to sample census data (e.g., may contain Rx information). In other related embodiments, the correlation/comparison includes comparing statistical data of a sample census pertinent to the categories of interest and values of the least one physiological parameter of the sample census. In such instances, the additional health information includes average physiological parameter values of the sample census that is set by the user, via the categories of interest, and may not include actual values corresponding to the user (e.g., may not contain Rx information). As previously described, the scale can (alternatively and/or in addition to a FUI or GUI) have a voice input/output circuitry that can obtain user's answers to questions via voice comments and inputs user information in response (e.g., a speaker component to capture voice sounds from the user and processing circuitry to recognize the voice commands and/or speech).

For example, if the categories of interest are demographic categories, the non-Rx health information includes various physiological parameter values of average users in the demographic categories and/or values of average users with a clinical indication that correlates to a physiological parameter of the user. Alternatively and/or in addition, the non-Rx health information includes general medical insights related to the categories of interest. For example, “Did you know if you are over the age of 55 and have gained 15 pounds, you are at risk for a particular disease/disorder?” The scale asks the user if the user would like to include this factor or disease in their categories of interest to dynamically update the categories of interest of the user.

Various categories of interest, in accordance with the present disclosure, include demographics of the user, disorders, disease, symptoms, prescription or non-prescription drugs, treatments, past medical history, family medical history, genetics, life style (e.g., exercise habits, eating habits, work environment), among other categories and combinations thereof. In a number of embodiments, various physiological factors are an indicator for a disease and/or disorder. For example, an increase in weight, along with other factors, can indicate an increased risk of atrial fibrillation. Further, atrial fibrillation is more common in men. In some instances, symptoms of a particular disorder are different for different categories of interest (e.g., symptoms of atrial fibrillation can be different between men and women). For example, in women, systolic blood pressure is associated with atrial fibrillation. In other instances, sleep apnea may be assessed via an ECG and is correlated to weight of the user. Furthermore, various cardiac conditions are assessed using an ECG. For example, atrial fibrillation can be characterized and/or identified in response to a user having no or fibrillating p-waves, no QRS complex, and no baseline/inconsistent beat fluctuations. Atrial flutter, by contrast, can be characterized by having no p-wave, variable heart rate, having QRS complexes, and a generally regular rhythm. Ventricular tachycardia (VT) can be characterized by a rate of greater than 120 beats per minute, and short or broad QRS complexes (depending on the type of VT). Atrio-Ventricular (AV) block can be characterized by PR intervals that are greater than normal (e.g., a normal range for an adult is generally 0.12 to 0.20 seconds), normal-waves, QRS complexes can be normal or prolong shaped, and the pulse can be regular (but slow at 20-40 beats per minute). For more specific and general information regarding atrial fibrillation and sleep apnea, reference is made herein to https://www.clevelandclinicmeded.com/medicalpubs/diseasemanagement/cardiology/atri al-fibrillation/and http://circ.ahajournals.org/content/118/10/1080.full, which are fully incorporated herein for its specific and general teachings. Further, other data and demographics that are known and/or are developed can be added and used to derive additional health information.

For example, the categories of interest for a particular user can include a change in weight, age 45-55, and female. The scale obtains raw data, including user weight, using the data-procurement circuitry and the remote user-physiologic device obtains raw data and the categories of interest from the user. The scale outputs the raw data to the remote user-physiologic device 109 or the remote user-physiologic device 109 outputs signals indicative of cardio-related physiologic data (responsive to activation of the communication). The remote user-physiologic device 109 and/or the scale correlates the categories of interest to the various raw data and derives non-Rx health information therefrom. Further, the remote user-physiologic device 109 and/or scale, over time, historically collects and correlates the categories of interest of the user and data from the data-procurement circuitry. The remote user-physiologic device 109 and/or the scale, in various embodiments, sends the data to a physician and/or non-Rx health information to the user (to print and/or otherwise view).

The remaining figures illustrate various ways to collect the physiologic data from the user, electrode configurations, and alternative modes of the processing circuitry 104. For general and specific information regarding the collection of physiologic data, electrode configurations, and alternative modes, reference is made to U.S. patent application Ser. No. 14/338,266 filed on Oct. 7, 2015, which is hereby fully incorporated by references for its teachings.

FIG. 1d shows current paths 100 through the body of a user 105 standing on a scale 110 for the IPG trigger pulse and Foot IPG, consistent with various aspects of the present disclosure. Impedance measurements 115 are measured when the user 105 is standing and wearing coverings over the feet (e.g., socks or shoes), within the practical limitations of capacitive-based impedance sensing, with energy limits considered safe for human use. The measurements 115 can be made with non-clothing material placed between the user's bare feet and contact electrodes, such as thin films or sheets of plastic, glass, paper or wax paper, whereby the electrodes operate within energy limits considered safe for human use. The IPG measurements can be sensed in the presence of callouses on the user's feet that normally diminish the quality of the signal.

As shown in FIG. 1d, the user 105 is standing on a scale 110, where the tissues of the user's body will be modeled as a series of impedance elements, and where the time-varying impedance elements change in response to cardiovascular and non-cardiovascular movements of the user. ECG and IPG measurements sensed through the feet can be challenging to take due to small impedance signals with (1) low SNR, and because they are (2) frequently masked or distorted by other electrical activity in the body such as the muscle firings in the legs to maintain balance. The human body is unsteady while standing still, and constant changes in weight distribution occur to maintain balance. As such, cardiovascular signals that are measured with weighing scale-based sensors typically yield signals with poor SNR, such as the Foot IPG and standing BCG. Thus, such scale-based signals require a stable and high quality synchronous timing reference, to segment individual heartbeat-related signals for signal averaging to yield an averaged signal with higher SNR versus respective individual measurements.

The ECG can be used as the reference (or trigger) signal to segment a series of heartbeat-related signals measured by secondary sensors (optical, electrical, magnetic, pressure, microwave, piezo, etc.) for averaging a series of heartbeat-related signals together, to improve the SNR of the secondary measurement. The ECG has an intrinsically high SNR when measured with body-worn gel electrodes, or via dry electrodes on handgrip sensors. In contrast, the ECG has a low SNR when measured using foot electrodes while standing on said scale platforms; unless the user is standing perfectly still to eliminate electrical noise from the leg muscles firing due to body motion. As such, ECG measurements at the feet while standing are considered to be an unreliable trigger signal (low SNR). Therefore, it is often difficult to obtain a reliable cardiovascular trigger reference timing when using ECG sensors incorporated in base scale platform devices. Both Inan, et al. (IEEE Transactions on Information Technology in Biomedicine, 14:5, 1188-1196, 2010) and Shin, et al. (Physiological Measurement, 30, 679-693, 2009) have shown that the ECG component of the electrical signal measured between the two feet while standing was rapidly overpowered by the electromyogram (EMG) signal resulting from the leg muscle activity involved in maintaining balance.

The accuracy of cardiovascular information obtained from weighing scales is also influenced by measurement time. The number of beats obtained from heartbeats for signal averaging is a function of measurement time and heart rate. Typically, a resting heart rates range from 60 to 100 beats per minute. Therefore, short signal acquisition periods may yield a low number of beats to average, which may cause measurement uncertainty, also known as the standard error in the mean (SEM). SEM is the standard deviation of the sample mean estimate of a population mean. Where, SE is the standard error in the samples N, which is related to the standard error or the population S.

$$SE = \frac{S}{\sqrt{N}}$$

For example, a five second signal acquisition period may yield a maximum of five to eight beats for ensemble averaging, while a 10 second signal acquisition could yield 10-16 beats. However, the number of beats available for averaging and SNR determination is usually reduced for the following factors; (1) truncation of the first and last ensemble beat in the recording by the algorithm, (2) triggering beats falsely missed by triggering algorithm, (3) cardiorespiratory variability, (4) excessive body motion corrupting the trigger and Foot IPG signal, and (5) loss of foot contact with the measurement electrodes.

Sources of noise can require multiple solutions for SNR improvements for the signal being averaged. Longer measurement times increase the number of beats lost to truncation, false missed triggering, and excessive motion. Longer measurement times also reduce variability from cardiorespiratory effects. If shorter measurement times (e.g., less than 30 seconds) are desired for scale-based sensor platforms, sensing improvements need to tolerate body motion and loss of foot contact with the measurement electrodes.

The human cardiovascular system includes a heart with four chambers, separated by valves that return blood to the heart from the venous system into the right side of the heart, through the pulmonary circulation to oxygenate the blood, which then returns to the left side of the heart, where the oxygenated blood is pressurized by the left ventricles and is pumped into the arterial circulation, where blood is distributed to the organs and tissues to supply oxygen. The cardiovascular or circulatory system is designed to ensure oxygen availability and is often the limiting factor for cell survival. The heart normally pumps five to six liters of blood every minute during rest and maximum cardiac output during exercise increases up to seven-fold, by modulating heart rate and stroke volume. The factors that affect heart rate include autonomic innervation, fitness level, age and hormones. Factors affecting stroke volume include heart size, fitness level, contractility or pre-ejection period, ejection duration, preload or end-diastolic volume, afterload or systemic resistance. The cardiovascular system is constantly adapting to maintain a homeostasis (set point) that minimizes the work done by the heart to maintain cardiac output. As such, blood pressure is continually adjusting to minimize work demands during rest. Cardiovascular disease encompasses a variety of abnormalities in (or that affect) the cardiovascular system that degrade the efficiency of the system, which include but are not limited to chronically elevated blood pressure, elevated cholesterol levels, edema, endothelial dysfunction, arrhythmias, arterial stiffening, atherosclerosis, vascular wall thickening, stenosis, coronary artery disease, heart attack, stroke, renal dysfunction, enlarged heart, heart failure, diabetes, obesity and pulmonary disorders.

Each cardiac cycle results in a pulse of blood being delivered into the arterial tree. The heart completes cycles of atrial systole, delivering blood to the ventricles, followed by ventricular systole delivering blood into the lungs and the systemic arterial circulation, where the diastole cycle begins. In early diastole the ventricles relax and fill with blood, then in mid-diastole the atria and ventricles are relaxed and the ventricles continue to fill with blood. In late diastole, the sinoatrial node (the heart's pacemaker) depolarizes then contracting the atria, the ventricles are filled with more blood and the depolarization then reaches the atrioventricular node and enters the ventricular side beginning the systole phase. The ventricles contract and the blood is pumped from the ventricles to arteries.

The ECG is the measurement of the heart's electrical activity and is described in five phases. The P-wave represents atrial depolarization, the PR interval is the time between the P-wave and the start of the QRS complex. The QRS wave complex represents ventricular depolarization. The QRS complex is the strongest wave in the ECG and is frequently used as a timing reference for the cardiovascular cycle. Atrial repolarization is masked by the QRS complex. The ST interval represents the period of zero potential between ventricular depolarization and repolarization. The cycle concludes with the T-wave representing ventricular repolarization.

The blood ejected into the arteries creates vascular movements due to the blood's momentum. The blood mass ejected by the heart first travels headward in the ascending aorta and travels around the aortic arch then travels down the descending aorta. The diameter of the aorta increases during the systole phase due to the high compliance (low stiffness) of the aortic wall. Blood traveling in the descending aorta bifurcates in the iliac branch which transitions into a stiffer arterial region due to the muscular artery composition of the leg arteries. The blood pulsation continues down the leg and foot. Along the way, the arteries branch into arteries of smaller diameter until reaching the capillary beds where the pulsatile blood flow turns into steady blood flow, delivering oxygen to the tissues. The blood returns to the venous system terminating in the vena cava, where blood returns to the right atrium of the heart for the subsequent cardiac cycle.

Surprisingly, high quality simultaneous recordings of the Leg IPG and Foot IPG are attainable in a practical manner (e.g., a user operating the device correctly simply by standing on the impedance body scale foot electrodes), and can be used to obtain reliable trigger fiducial timings from the Leg IPG signal. This acquisition can be far less sensitive to motion-induced noise from the Leg EMG that often compromises Leg ECG measurements. Furthermore, it has been discovered that interleaving the two Kelvin electrode pairs for a single foot, result in a design that is insensitive to foot placement within the boundaries of the overall electrode area. As such, the user is not constrained to comply with accurate foot placement on conventional single foot Kelvin arrangements, which are highly prone to introducing motion artifacts into the IPG signal, or result in a loss of contact if the foot is slightly misaligned. Interleaved designs begin when one or more electrode surfaces cross over a single imaginary boundary line separating an excitation and sensing electrode pair. The interleaving is configured to maintain uniform foot surface contact area on the excitation and sensing electrode pair, regardless of the positioning of the foot over the combined area of the electrode pair.

Various aspects of the present disclosure include a weighing scale platform (e.g., scale 110) of an area sufficient for an adult of average size to stand comfortably still and minimize postural swaying. The nominal scale length (same orientation as foot length) is 12 inches and the width is 12 inches. The width can be increased to be consistent with the feet at shoulder width or slightly broader (e.g., 14 to 18 inches, respectively).

FIG. 1e is a flow chart depicting an example manner in which a user-specific physiologic meter or scale may be programmed in accordance with the present disclosure. This flow chart uses a computer processor circuit (or CPU) along with a memory circuit shown herein as user profile memory 146a. The CPU operates in a low-power consumption mode, which may be in off mode or a low-power sleep mode, and at least one other higher power consumption mode of operation. The CPU can be integrated with presence and/or motion sense circuits, such as a passive infrared (PIR) circuit and/or pyroelectric PIR circuit. In a typical application, the PIR circuit provides a constant flow of data indicative of amounts of radiation sensed in a field of view directed by the PIR circuit. For instance, the PIR circuit can be installed behind an upper surface which is transparent to infrared light (and/or other visible light) of the platform and installed at an angle so that the motion of the user approaching the platform apparatus is sensed. Radiation from the user, upon reaching a certain detectable level, wakes up the CPU which then transitions from the low-power mode, as depicted in block 140, to a regular mode of operation. Alternatively, the low-power mode of operation is transitioned from a response to another remote/wireless input used as a presence to awaken the CPU. In other embodiments, user motion can be detected by an accelerometer integrated in the scale or the motion is sensed with a single integrated microphone or microphone array, to detect the sounds of a user approaching.

Accordingly, from block 140, flow proceeds to block 142 where the user or other intrusion is sensed as data received at the platform apparatus. At block 144, the circuitry assesses whether the received data qualifies as requiring a wake up. If not, flow turns to block 140. If however, wake up is required, flow proceeds from block 144 to block 146 where the CPU assesses whether a possible previous user has approached the platform apparatus. This assessment is performed by the CPU accessing the user profile memory 146A and comparing data stored therein for one or more such previous users with criteria corresponding to the received data that caused the wake up. Such criteria includes, for example, the time of the day, the pace at which the user approached the platform apparatus as sensed by the motion detection circuitry, the height of the user as indicated by the motion sensing circuitry and/or a camera installed and integrated with the CPU, and/or more sophisticated bio-metric data provided by the user and/or automatically by the circuitry in the platform apparatus.

As discussed herein, such sophisticated circuitry can include one or more of the following user-specific attributes: foot length, type of foot arch, weight of user, and/or manner and speed at which the user steps onto the platform apparatus or by user speech (e.g., voice). In some embodiments, facial or body-feature recognition may also be used in connection with the camera and comparisons of images therefrom to images in the user profile memory.

From block 146, flow proceeds to block 148 where the CPU obtains and/or updates user corresponding data in the user profile memory. As a learning program is developed in the user profile memory, each access and use of the platform apparatus is used to expand on the data and profile for each such user. From block 148, flow proceeds to block 150 where a decision is made regarding whether the set of electrodes at the upper surface of the platform are ready for the user, such as may be based on the data obtained from the user profile memory. For example, delays may ensue from the user moving his or her feet about the upper surface of the platform apparatus, as may occur while certain data is being retrieved by the CPU (whether internally or from an external source such as a program or configuration data updates from the Internet cloud) or when the user has stepped over the user-display. If the electrodes are not ready for the user, flow proceeds from block 150 to block 152 to accommodate this delay.

Once the CPU determines that the electrodes are ready for use while the user is standing on the platform surface, flow proceeds to block 160. Stabilization of the user on the platform surface may be ascertained by injecting current through the electrodes via the interleaved arrangement thereof. Where such current is returned via other electrodes for a particular foot and/or foot size, and is consistent for a relatively brief period of time, for example, a few seconds, the CPU can assume that the user is standing still and ready to use the electrodes and related circuitry. At block 160, a decision is made that both the user and the platform apparatus are ready for measuring impedance and certain segments of the user's body, including at least one foot.

The remaining flow of FIG. 1e includes the application and sensing of current through the electrodes for finding the optimal electrodes (162) and for performing impedance measurements (block 164). These measurements are continued until completed at block 166 and all such useful measurements are recorded and are logged in the user profile memory for this specific user, at block 168. At block 172, the CPU generates output data to provide feedback as to the completion of the measurements and, as can be indicated as a request via the user profile for this user, as an overall report on the progress for the user and relative to previous measurements made for this user has stored in the user profile memory. Such feedback may be shown on the user-display, through a speaker with co-located apertures in the platform for audible reception by the user, and/or by vibration circuitry which, upon vibration under control of the CPU, the user can sense through one or both feet while standing on the scale. From this output at block 172, flow returns to the low power mode as indicated at block 174 with the return to the beginning of the flow at the block 140.

FIG. 2a shows an example of the insensitivity to foot placement 200 on scale electrode pairs 205/210 with multiple excitation paths 220 and sensing current paths 215, consistent with various aspects of the present disclosure. An aspect of the platform is that it has a thickness and strength to support a human adult of at least 200 pounds without fracturing, and another aspect of the device platform is comprised of at least six electrodes, where the first electrode pair 205 is solid and the second electrode pair 210 are interleaved. Another aspect is the first and second interleaved electrode pairs 205/210 are separated by a distance of at least 40+/−5 millimeters, where the nominal separation of less than 40 millimeters has been shown to degrade the single Foot IPG signal. Another key aspect is the electrode patterns are made from materials with low resistivity such as stainless steel, aluminum, hardened gold, ITO, index matched ITO (IMITO), carbon printed electrodes, conductive tapes, silver-impregnated carbon printed electrodes, conductive adhesives, and similar materials with resistivity lower than 300 ohms/sq. The resistivity can be below 150 ohms/sq. The electrodes are connected to the electronic circuitry in the scale by routing the electrodes around the edges of the scale to the surface below, or through at least one hole in the scale (e.g., a via hole).

Suitable electrode arrangements for dual Foot IPG measurements can be realized in other embodiments. In certain embodiments, the interleaved electrodes are patterned on the reverse side of a thin piece (e.g., less than 2 mm) of high-ion-exchange (HIE) glass, which is attached to a scale substrate and used in capacitive sensing mode. In certain embodiments, the interleaved electrodes are patterned onto a thin piece of paper or plastic which can be rolled up or folded for easy storage. In certain embodiments, the interleaved electrodes are integrated onto the surface of a tablet computer for portable IPG measurements. In certain embodiments, the interleaved electrodes are patterned onto a kapton substrate that is used as a flex circuit.

In certain embodiments, the scale area has a length of 10 inches with a width of eight inches for a miniature scale platform. Alternatively, the scale may be larger (up to 36 inches wide) for use in bariatric class scales.

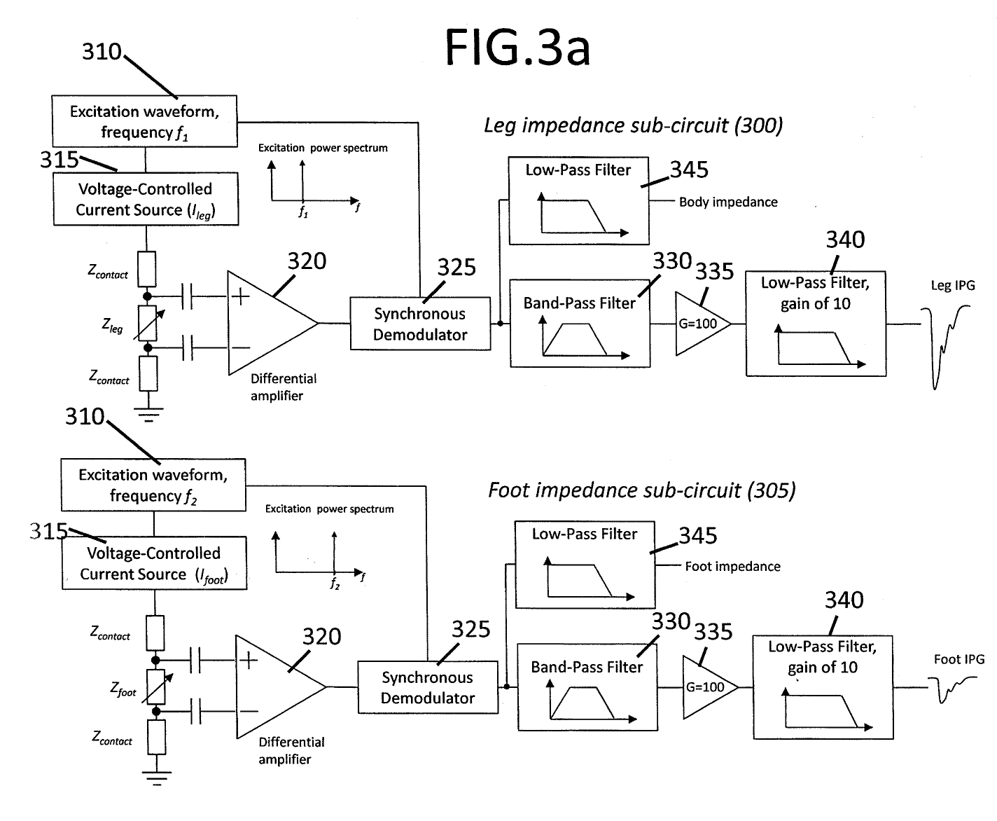

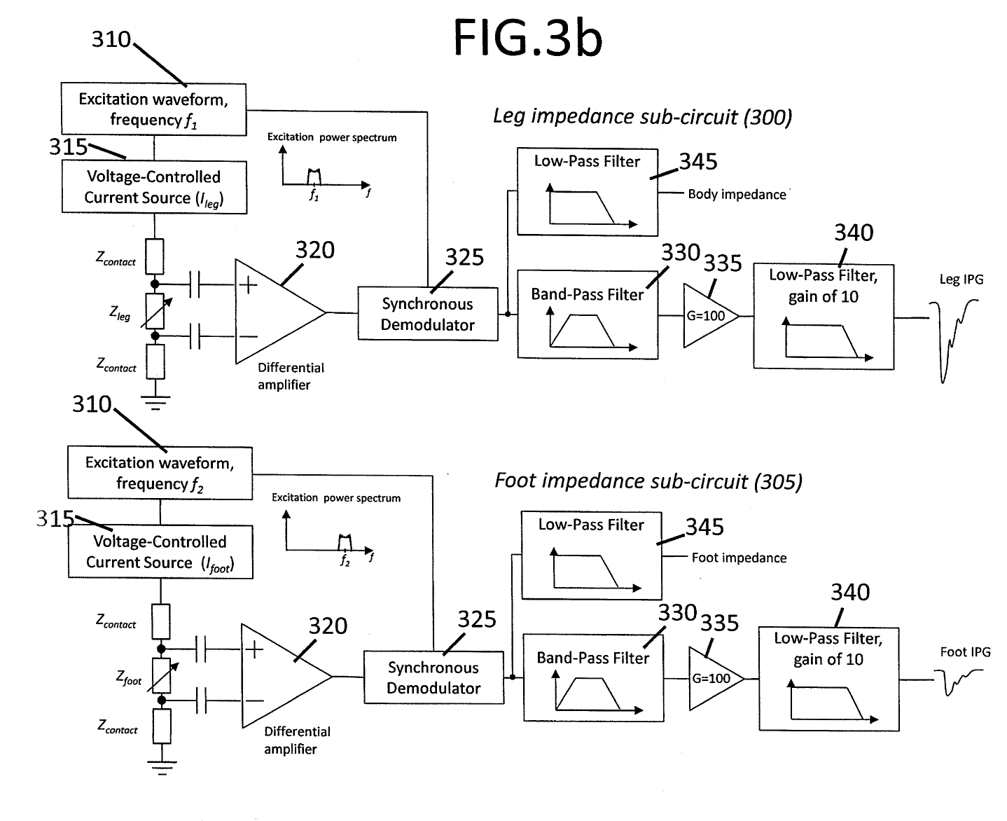

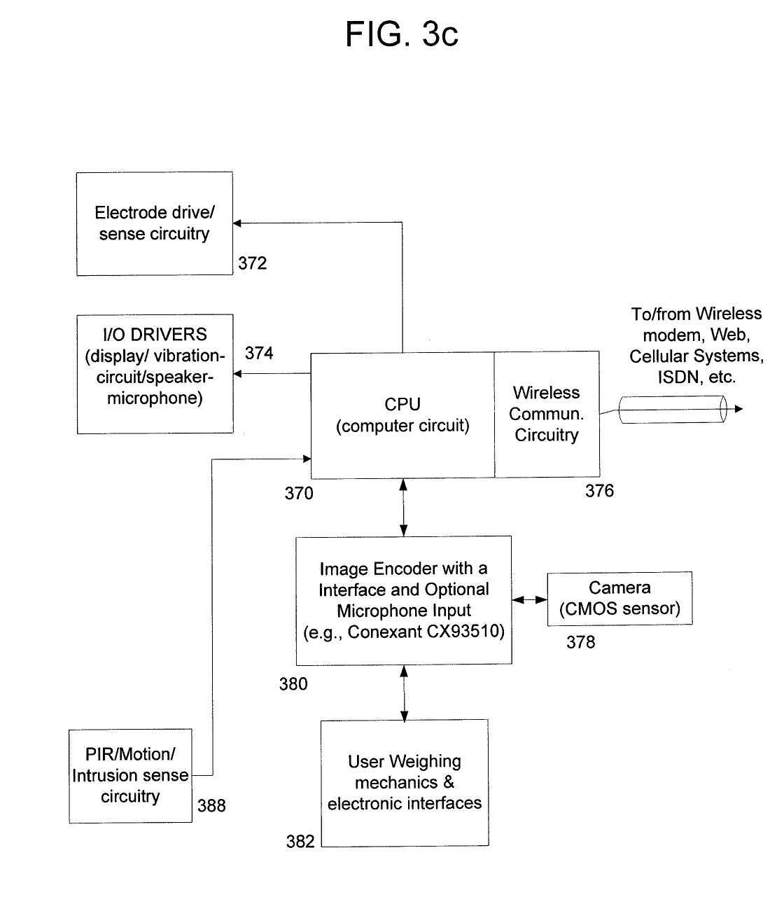

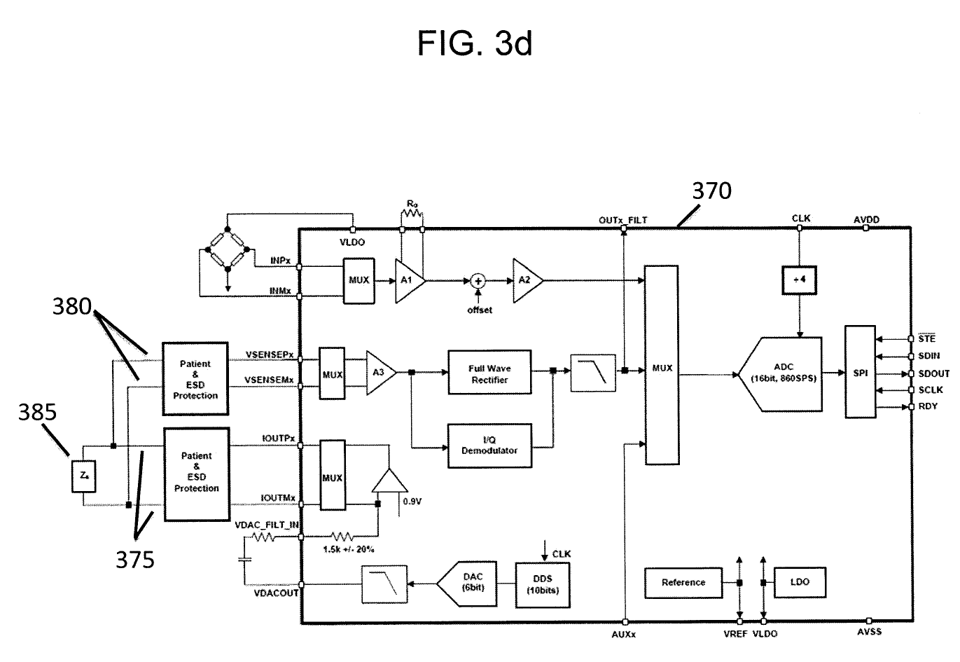

In the present disclosure, the leg and foot impedance measurements can be simultaneously carried out using a multi-frequency approach, in which the leg and foot impedances are excited by currents modulated at two or more different frequencies, and the resulting voltages are selectively measured using a synchronous demodulator as shown in FIG. 3a. This homodyning approach can be used to separate signals (in this case, the voltage drop due to the imposed current) with very high accuracy and selectivity.

This measurement configuration is based on a four-point configuration in order to minimize the impact of the contact resistance between the electrode and the foot, a practice well-known in the art of impedance measurement. In this configuration the current is injected from a set of two electrodes (the “injection” and “return” electrodes), and the voltage drop resulting from the passage of this current through the resistance is sensed by two separate electrodes (the “sense” electrodes), usually located in the path of the current. Since the sense electrodes are not carrying any current (by virtue of their connection to a high-impedance differential amplifier), the contact impedance does not significantly alter the sensed voltage.

In order to sense two distinct segments of the body (the legs and the foot), two separate current paths are defined by electrode positioning. Therefore two injection electrodes are used, each connected to a current source modulated at a different frequency. The injection electrode for leg impedance is located under the plantar region of the left foot, while the injection electrode for the Foot IPG is located under the heel of the right foot. Both current sources share the same return electrode located under the plantar region of the right foot. This is an illustrative example. Other configurations may be used.

The sensing electrodes can be localized so as to sense the corresponding segments. Leg IPG sensing electrodes are located under the heels of each foot, while the two foot sensing electrodes are located under the heel and plantar areas of the right foot. The inter-digitated nature of the right foot electrodes ensures a four-point contact for proper impedance measurement, irrespectively of the foot position, as already explained.

FIG. 2b shows an example of electrode configurations, consistent with various aspects of the disclosure. As shown by the electrode connections, in some embodiments, ground is coupled to the heel of one foot of the user (e.g., the right foot) and the foot current injection (e.g., excitation paths 220) is coupled to the toes of the respective one foot (e.g., toes of the right foot). The leg current injection is coupled to the toes of the other foot (e.g., toes of the left foot).

FIG. 2c shows an example of electrode configurations, consistent with various aspects of the disclosure. As shown by the electrode connections, in some embodiments, ground is coupled to the heel of one foot of the user (e.g., the right foot) and the foot current injection (e.g., excitation paths 220) is coupled to the toes of the one foot (e.g., toes of the right foot). The leg current injection is coupled to the heels of the other foot of the user (e.g., heels of the left foot).

FIGS. 3a-3b show example block diagrams depicting the circuitry for sensing and measuring the cardiovascular time-varying IPG raw signals and steps to obtain a filtered IPG waveform, consistent with various aspects of the present disclosure. The example block diagrams shown in FIGS. 3a-3b are separated in to a leg impedance sub-circuit 300 and a foot impedance sub-circuit 305.

Excitation is provided by way of an excitation waveform circuit 310. The excitation waveform circuit 310 provides a stable amplitude excitation signal by way of various wave shapes of various, frequencies, such as more specifically, a sine wave signal (as is shown in FIG. 3a) or, more specifically, a square wave signal (as shown in FIG. 3b). This excitation waveform (of sine, square, or other wave shape) is fed to a voltage-controlled current source circuit 315 which scales the signal to the desired current amplitude. The generated current is passed through a decoupling capacitor (for safety) to the excitation electrode, and returned to ground through the return electrode (grounded-load configuration). Amplitudes of 1 and 4 mA peak-to-peak are typically used for Leg and Foot IPGs, respectively.

The voltage drop across the segment of interest (legs or foot) is sensed using an instrumentation differential amplifier (e.g., Analog Devices AD8421) 320. The sense electrodes on the scale are AC-coupled to the inputs of the differential amplifier 320 (configured for unity gain), and any residual DC offset is removed with a DC restoration circuit (as exemplified in Burr-Brown App Note Application Bulletin, SBOA003, 1991, or Burr-Brown/Texas Instruments INA118 datasheet). Alternatively, a fully differential input amplification stage can be used which eliminates the need for DC restoration.

The signal is then demodulated with a phase-sensitive synchronous demodulator circuit 325. The demodulation is achieved in this example by multiplying the signal by 1 or −1 synchronously in-phase with the current excitation. Such alternating gain is provided by an operational amplifier (op amp) and an analog switch (SPST), such as an ADG442 from Analog Devices). More specifically, the signal is connected to both positive and negative inputs through 10 kOhm resistors. The output is connected to the negative input with a 10 kOhm resistor as well, and the switch is connected between the ground and the positive input of the op amp. When open, the gain of the stage is unity. When closed (positive input grounded), the stage acts as an inverting amplifier with a gain of −1. Further, fully differential demodulators can alternatively be used which employ pairs of DPST analog switches whose configuration can provide the benefits of balanced signals and cancellation of charge injection artifacts. Alternatively, other demodulators such as analog multipliers or mixers can be used. The in-phase synchronous detection allows the demodulator to be sensitive to only the real, resistive component of the leg or foot impedance, thereby rejecting any imaginary, capacitive components which may arise from parasitic elements associated with the foot to electrode contacts.

Once demodulated, the signal is band-pass filtered (0.4-80 Hz) with a band-pass filter circuit 330 before being amplified with a gain of 100 with a non-inverting amplifier circuit 335 (e.g., using an LT1058 operational amplifier from Linear Technology Inc.). The amplified signal is further amplified by 10 and low-pass filtered (cut-off at 20 Hz) using a low-pass filter circuit 340 such as 2-pole Sallen-Key filter stage with gain. The signal is then ready for digitization and further processing. In certain embodiments, the signal from the demodulator circuit 325 can be passed through an additional low-pass filter circuit 345 to determine body or foot impedance.

In certain embodiments, the generation of the excitation voltage signal, of appropriate frequency and amplitude, is carried out by a microcontroller, such as an MSP430 (Texas Instruments, Inc.) or a PIC18Fxx series (Microchip Technology, Inc.). The voltage waveform can be generated using the on-chip timers and digital input/outputs or pulse width modulation (PWM) peripherals, and scaled down to the appropriate voltage through fixed resistive dividers, active attenuators/amplifiers using on-chip or off-chip operational amplifiers, as well as programmable gain amplifiers or programmable resistors. In certain embodiments, the generation of the excitation frequency signal can be accomplished by an independent quartz crystal oscillator whose output is frequency divided down by a series of toggle flip-flops (such as an ECS-100AC from ECS International, Inc., and a CD4024 from Texas Instruments, Inc.). In certain embodiments, the generation of the wave shape and frequency can be accomplished by a direct digital synthesis (DDS) integrated circuit (such as an AD9838 from Analog Devices, Inc.). In certain embodiments, the generation of the wave shape (either sine or square) and frequency can be accomplished by a voltage-controlled oscillator (VCO) which is controlled by a digital microcontroller, or which is part of a phase-locked loop (PLL) frequency control circuit. Alternatively, the waveforms and frequencies can be directly generated by on- or off-chip digital-to-analog converters (DACs).

In certain embodiments, the shape of the excitation is not square, but sinusoidal. Such configuration can reduce the requirements on bandwidth and slew rate for the current source and instrumentation amplifier. Harmonics, potentially leading to higher electromagnetic interference (EMI), can also be reduced. Such excitation may also reduce electronics noise on the circuit itself. Lastly, the lack of harmonics from sine wave excitation may provide a more flexible selection of frequencies in a multi-frequency impedance system, as excitation waveforms have fewer opportunities to interfere between each other. Due to the concentration of energy in the fundamental frequency, sine wave excitation could also be more power-efficient. In certain embodiments, the shape of the excitation is not square, but trapezoidal. Alternatively, raised cosine pulses (RCPs) could be used as the excitation wave shape, providing an intermediate between sine and square waves. RCPs could provide higher excitation energy content for a given amplitude, but with greatly reduced higher harmonics.

To further reduce potential electromagnetic interference (EMI), other strategies may be used, such as by dithering the square wave signal (i.e., introducing jitter in the edges following a fixed or random pattern) which leads to so-called spread spectrum signals, in which the energy is not localized at one specific frequency (or a set of harmonics), but rather distributed around a frequency (or a set of harmonics). Because of the synchronous demodulation scheme, phase-to-phase variability introduced by spread-spectrum techniques will not affect the impedance measurement. Such a spread-spectrum signal can be generated by, but not limited to, specialized circuits (e.g., Maxim MAX31C80, SiTime SiT9001), or generic microcontrollers (see Application Report SLAA291, Texas Instruments, Inc.). These spread-spectrum techniques can be combined with clock dividers to generate lower frequencies as well.

As may be clear to one skilled in the art, these methods of simultaneous measurement of impedance in the leg and foot can be used for standard Body Impedance Analysis (BIA), aiming at extracting the relative content of total water, free-water, fat mass and other body composition measures. Impedance measurements for BIA are typically done at frequencies ranging from kilohertz up to several megahertz. The multi-frequency synchronous detection measurement methods described above can readily be used for such BIA, provided that low-pass filtering (345, FIGS. 3a and 3b) instead of band-pass filtering (330, FIGS. 3a and 3b) is performed following the demodulation. In certain embodiments, a separate demodulator channel may be driven by the quadrature phase of the excitation signal to allow the imaginary component of the body impedance to be extracted in addition to the real component. A more accurate BIA can be achieved by measuring both the real and imaginary components of the impedance. This multi-frequency technique can be combined with traditional sequential measurements used for BIA, in which the impedance is measured at several frequencies sequentially. These measurements are repeated in several body segments for segmental BIAs, using a switch matrix to drive the current into the desired body segments.

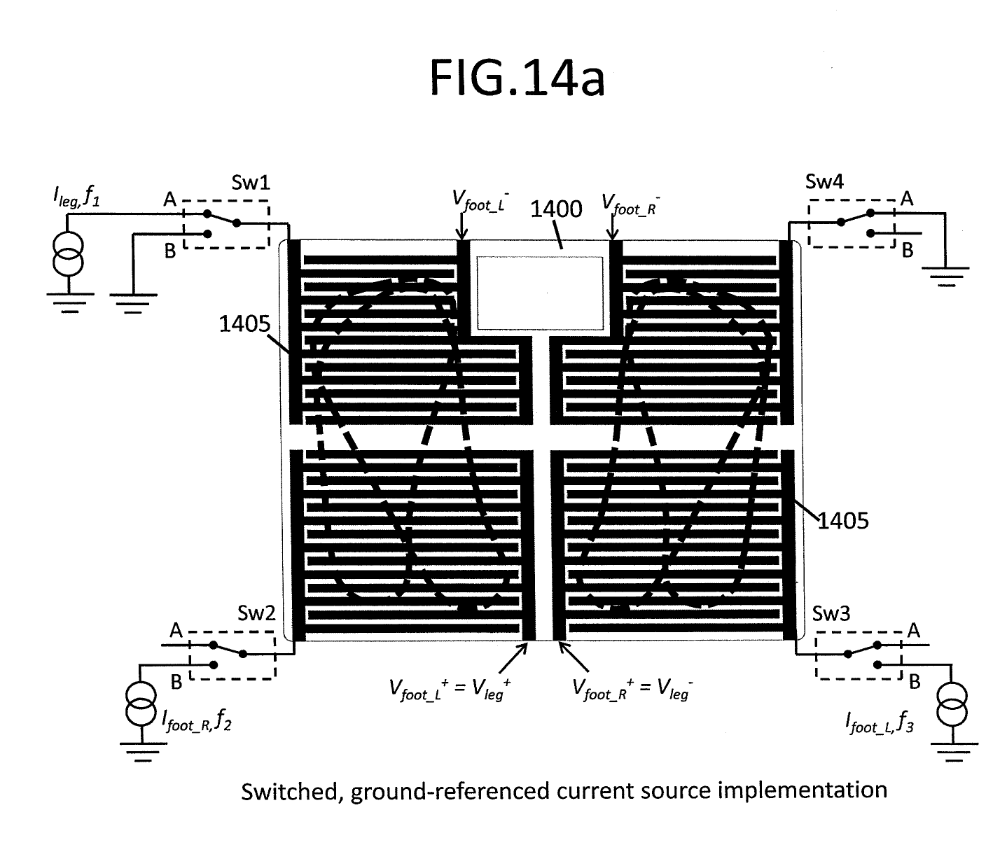

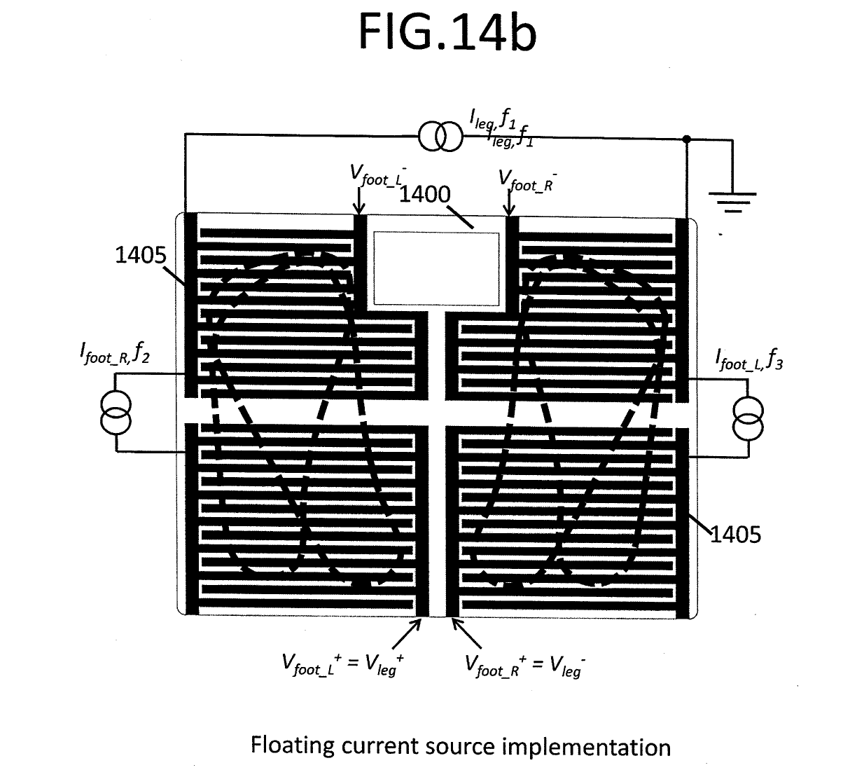

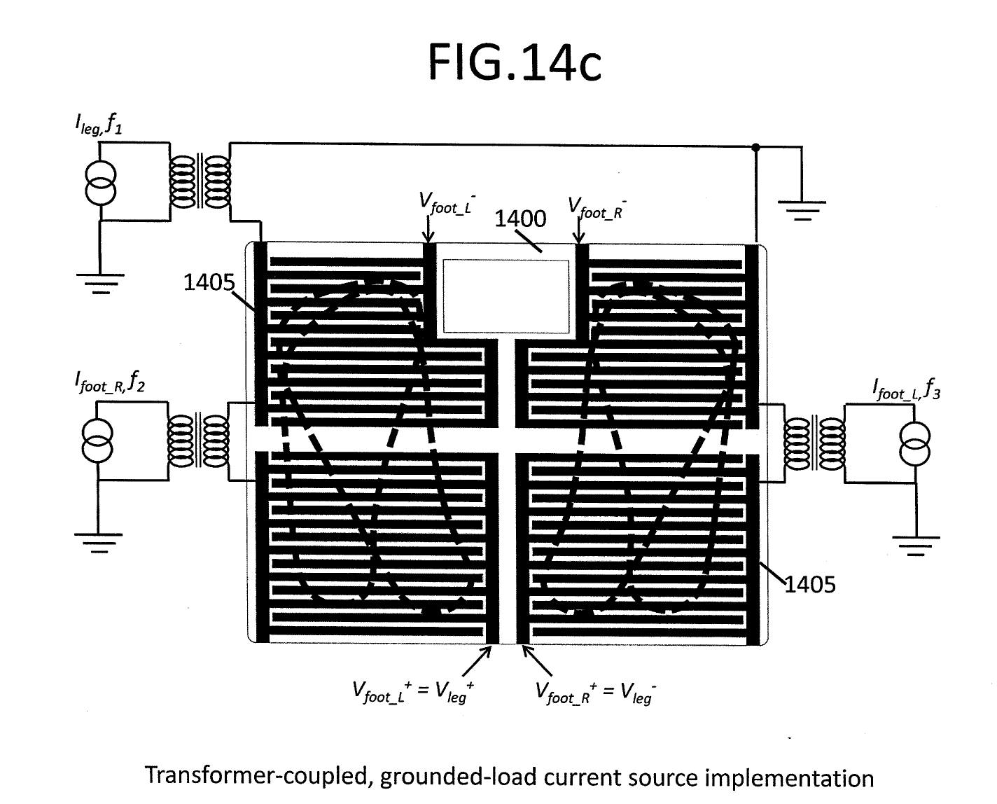

While FIG. 2a shows a circuit and electrode configuration suitable to measure two different segments (legs and one foot), this approach is not readily extendable to more segments due to the shared current return electrode (ground). To overcome this limitation, and provide simultaneous measurements in both feet, the system can be augmented with analog switches to provide time-multiplexing of the impedance measurements in the different segments. This multiplexing can be a one-time sequencing (each segment is measured once), or interleaved at a high-enough frequency that the signal can be simultaneously measured on each segment. The minimum multiplexing rate for proper reconstruction is twice the bandwidth of the measured signal, based on signal processing theory (the Nyquist rate), which equals to about 100 Hz for the impedance signal considered here. The rate must also allow for the signal path to settle in between switching, which usually limits the maximum multiplexing rate. Referring to FIG. 14a, one cycle might start the measurement of the leg impedance and left foot impedances (similarly to previously described, sharing a common return electrode), but then follow with a measurement of the right foot after reconfiguring the switches. For specific information regarding typical switch configurations, reference to U.S. patent application Ser. No. 14/338,266 filed on Oct. 7, 2015, which is fully incorporated for its specific and general teaching of switch configurations.

Since right and left feet are measured sequentially, one should note that a unique current source (at the same frequency) may be used to measure both, providing that the current source is not connected to the two feet simultaneously through the switches, in which case the current would be divided between two paths. One should also note that a fully-sequential measurement, using a single current source (at a single frequency) successively connected to the three different injection electrodes, could be used as well, with the proper switch configuration sequence (no splitting of the current path).

In certain embodiments, the measurement of various body segments, and in particular the legs, right foot and left foot, is achieved simultaneously due to as many floating current sources as segments to be measured, running at separate frequency so they can individually be demodulated. Such configuration is exemplified in FIG. 14b for three segments (legs, right and left feet). Such configuration has the advantage to provide true simultaneous measurements without the added complexity of time-multiplexing/demultiplexing, and associated switching circuitry. An example of such a floating current source is found in Plickett, et al., Physiological Measurement, 32 (2011). Another approach to floating current sources is the use of transformer-coupled current sources (as depicted in FIG. 14c). Using transformers to inject current into the electrodes enables the use of simpler, grounded-load current sources on the primary, while the electrodes are connected to the secondary. The transformer turns ratio can typically be 1:1, and since frequencies of interest for impedance measurement are typically in the 10-1000 kHz (occasionally 1 kHz for BIA), relatively small pulse transformers can be used. In order to limit the common mode voltage of the body, one of the electrodes in contact with the foot can be grounded.

While certain embodiments presented in the above specification have used current sources for excitation, the excitation can also be performed by a voltage source, where the resulting injection current is monitored by a current sense circuit so that impedance can still be derived by the ratio of the sensed voltage (on the sense electrodes) over the sensed current (injected in the excitation electrodes). It should be noted that broadband spectroscopy methods could also be used for measuring impedances at several frequencies. Combined with time-multiplexing and current switching described above, multi-segment broadband spectroscopy can be achieved.

Various aspects of the present disclosure are directed toward robust timing extraction of the blood pressure pulse in the foot which is achieved by means of a two-step processing. In a first step, the usually high-SNR Leg IPG is used to derive a reference (trigger) timing for each heart pulse. In a second step, a specific timing in the lower-SNR Foot IPG is extracted by detecting its associated feature within a restricted window of time around the timing of the Leg IPG.

Consistent with yet further embodiments of the present disclosure, FIG. 3c depicts an example block diagram of circuitry for operating core circuits and modules, including, for example, the operation of the CPU as in FIG. 1a with the related more specific circuit blocks/modules in FIGS. 3A-3B. As shown in the center of FIG. 3c, the computer circuit 370 is shown with other previously-mentioned circuitry in a generalized manner without showing some of the detailed circuitry (e.g., amplification and current injection/sensing (372)). The computer circuit 370 can be used as a control circuit with an internal memory circuit (or as integrated with the memory circuit for the user profile memory 146A of FIG. 1a) for causing, processing and/or receiving sensed input signals as at block 372. As discussed, these sensed signals can be responsive to injection current and/or these signals can be sensed by less complex grid-based sense circuitry surrounding the platform as is convention in capacitive touch-screen surfaces which, in certain embodiments, the platform includes.

As noted, the memory circuit can be used not only for the user profile memory, but also as to provide configuration and/or program code and/or other data such as user-specific data from another authorized source such as from a user monitoring his/her logged data and/or profile from a remote desk-top. The remote device or desk-top can communicate with and access such data via a wireless communication circuit 376. For example, the wireless communication circuit 376 provides an interface between an app on the user's cellular telephone/tablet and the apparatus, wherefrom the IPhone is the output/input interface for the platform (scale) apparatus including, for example, an output display, speaker and/or microphone, and vibration circuitry; each of these I/O aspects and components being discussed herein in connection with other example embodiments.

A camera 378 and image encoder circuit 380 (with compression and related features) can also be incorporated as an option. As discussed above, the weighing scale components, as in block 382, are also optionally included in the housing which encloses and/or surrounds the platform.

For long-lasting battery life in the platform apparatus (batteries not shown), at least the CPU 370, the wireless communication circuit 376, and other current draining circuits are inactive unless and until activated in response to the intrusion/sense circuitry 388. As shown, one specific implementation employs a Conexant chip (e.g., CX93510) to assist in the low-power operation. This type of circuitry is designed for motion sensors configured with a camera for visual verification and image and video monitoring applications (such as by supporting JPEG and MJPEG image compression and processing for both color and black and white images). When combined with an external CMOS sensor, the chip retrieves and stores compressed JPEG and audio data in an on-chip memory circuit (e.g., 256 KB/128 KB frame buffer) to alleviate the necessity of external memory. The chip uses a simple register set via the microprocessor interface and allows for wide flexibility in terms of compatible operation with another microprocessor.

In one specific embodiment, a method of using the platform with the plurality of electrodes are concurrently contacting a limb of the user, includes operating such to automatically obtain measurement signals from the plurality of electrodes. As noted above, these measurement signals might initially be through less complex (e.g., capacitive grid-type) sense circuitry. Before or while obtaining a plurality of measurement signals by operating the circuitry, the signal-sense circuitry 388 is used to sense wireless-signals indicative of the user approaching the platform and, in response, causing the CPU circuitry 370 to transition from a reduced power-consumption mode of operation and at least one higher power-consumption mode of operation. After the circuitry is operating in the higher power-consumption mode of operation, the CPU accesses the user-corresponding data stored in the memory circuit and causes a plurality of impedance-measurement signals to be obtained by using the plurality of electrodes while they are contacting the user via the platform; therefrom, the CPU generates signals corresponding to cardiovascular timings of the user.

The signal-sense circuit can be employed as a passive infrared detector and with the CPU programmed (as a separate module) to evaluate whether radiation from the passive infrared detector is indicative of a human. For example, sensed levels of radiation that corresponds to a live being, such as a dog, that is less than a three-foot height, and/or has not moved for more than a couple seconds, can be assessed as being a non-human.

Accordingly, as the user is recognized as being human, the CPU is activated and begins to attempt the discernment process of which user might be approaching. This is performed by the CPU accessing the user-corresponding data stored in the memory circuit (the user profile memory). If the user is recognized based on parameters such as discussed above (e.g., time of morning, speed of approach, etc.), the CPU can also select one of a plurality of different types of user-discernible visual/audible/tactile information and for presenting the discerned user with visual/audible/tactile information that was retrieved from the memory as being specific to the user. For example, user-selected visual/audible data can be outputted for the user. Also, responsive to the motion detection indication, the camera can be activated to capture at least one image of the user while the user is approaching the platform (and/or while the user is on the platform to log confirmation of the same user with the measured impedance information). As shown in block 374 of FIG. 3c, where a speaker is also integrated with the CPU, the user can simply command the platform apparatus to start the process and activation proceeds. As previously discussed, the scale can include voice input/output circuitry to receive the user commands via voice commands.

In another method, the circuitry of FIG. 3c is used with the electrodes being interleaved and engaging the user, as a combination weighing scale (via block 382) and a physiologic user-specific impedance-measurement device. By using the impedance-measurement signals and obtaining at least two impedance-measurement signals between one foot of the user and another location of the user, the interleaved electrodes assist the CPU in providing measurement results that indicate one or more of the following user-specific attributes as being indicative or common to the user: foot impedance, foot length, and type of arch, and wherein one or more of the user-specific attributes are accessed in the memory circuit and identified as being specific to the user. This information can be later retrieved by the user, medical and/or security personnel, according to a data-access authorization protocol as might be established upon initial configuration for the user.

FIG. 3d shows an exemplary block diagram depicting the circuitry for interpreting signals received from electrodes (e.g., 372 of FIG. 3c), and/or CPU 370 of FIG. 3c. The input electrodes 375 transmit electrical signals through the patient's body (depending on the desired biometric and physiological test to be conducted) and output electrodes 380 receive the modified signal as affected by a user's electrical impedance 385. Once received by the output electrodes 380, the modified signal is processed by processor circuitry 370 based on the selected test. Signal processing conducted by the processor circuitry 370 is discussed in more detail above (with regard to FIGS. 3a-b). In certain embodiments of the present disclosure, the circuitry within 370 is provided by Texas Instruments part # AFE4300.

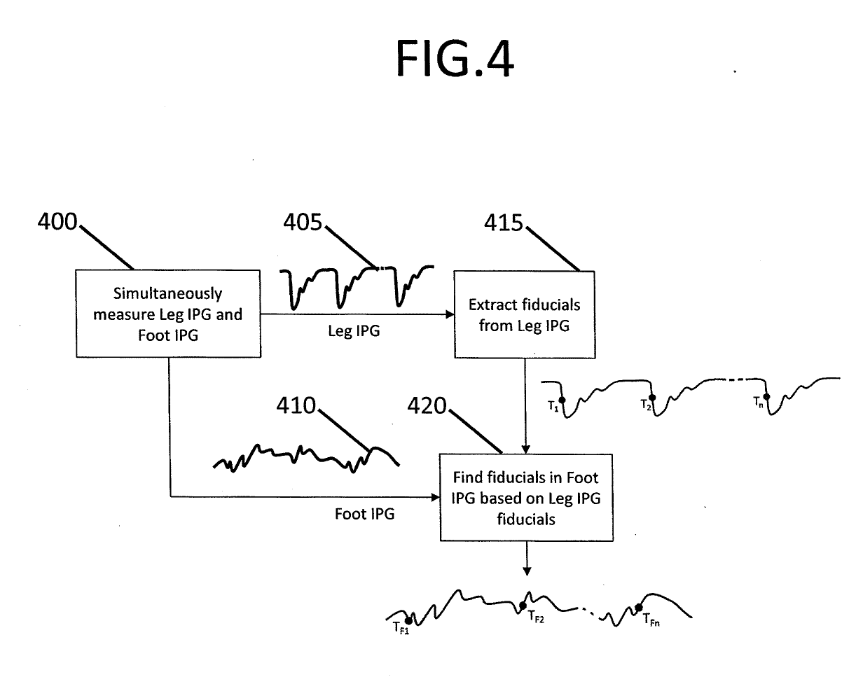

FIG. 4 shows an example block diagram depicting signal processing steps to obtain fiducial references from the individual Leg IPG “beats,” which are subsequently used to obtain fiducials in the Foot IPG, consistent with various aspects of the present disclosure. In the first step, as shown in block 400, the Leg IP and the Foot IPG are simultaneously measured. As shown at 405, the Leg IPG is low-pass filtered at 20 Hz with an 8-pole Butterworth filter, and inverted so that pulses have an upward peak. The location of the pulses is then determined by taking the derivative of this signal, integrating over a 100 ms moving window, zeroing the negative values, removing the large artifacts by zeroing values beyond 15× the median of the signal, zeroing the values below a threshold defined by the mean of the signal, and then searching for local maxima. Local maxima closer than a defined refractory period of 300 ms to the preceding ones are dismissed. The result is a time series of pulse reference timings.

As is shown in 410, the foot IPG is low-pass filtered at 25 Hz with an 8-pole Butterworth filter and inverted (so that pulses have an upward peak). Segments starting from the timings extracted (415) from the Leg IPG (reference timings) and extending to 80% of the previous pulse interval, but no longer than one second, are defined in the Foot IPG. This defines the time windows where the Foot IPG is expected to occur, avoiding misdetection outside of these windows. In each segment, the derivative of the signal is computed, and the point of maximum positive derivative (maximum acceleration) is extracted. The foot of the IPG signal is then computed using an intersecting tangent method, where the fiducial (420) is defined by the intersection between a first tangent to the IPG at the point of maximum positive derivative and a second tangent to the minimum of the IPG on the left of the maximum positive derivative within the segment.

The time series resulting from this two-step extraction is used with another signal to facilitate further processing. These timings are used as reference timings to improve the SNR of BCG signals to extract intervals between a timing of the BCG (typically the I-wave) and the Foot IPG for the purpose of computing the PWV, as previously disclosed in U.S. 2013/0310700 (Wiard). In certain embodiments, the timings of the Leg IPG are used as reference timings to improve the SNR of BCG signals, and the foot IPG timings are used to extract intervals between timing fiducials of the improved BCG (typically the I-wave) and the Foot IPG for the purpose of computing the PTT and the (PWV).

In certain embodiments, the processing steps include an individual pulse SNR computation after individual timings are extracted, either in Leg IPG or Foot IPG. Following the computation of the SNRs, pulses with a SNR below a threshold value are eliminated from the time series, to prevent propagating noise. The individual SNRs may be computed in a variety of methods known to one skilled in the art. For instance, an estimated pulse can be computed by ensemble averaging segments of signal around the pulse reference timing. The noise associated with each pulse is defined as the difference between the pulse and the estimated pulse. The SNR is the ratio of the root-mean-square (RMS) value of the estimated pulse over the RMS value of the noise for that pulse.

In certain embodiments, the time interval between the Leg IPG pulses, and the Foot IPG pulses, also detected by the above-mentioned methods, is extracted. The Leg IPG measuring a pulse occurring earlier in the legs compared to the pulse from the Foot IPG, the interval between these two is related to the propagation speed in the lower body, i.e., the peripheral vasculature. This provides complementary information to the interval extracted between the BCG and the Foot IPG for instance, and is used to decouple central versus peripheral vascular properties. It is also complementary to information derived from timings between the BCG and the Leg ICG.

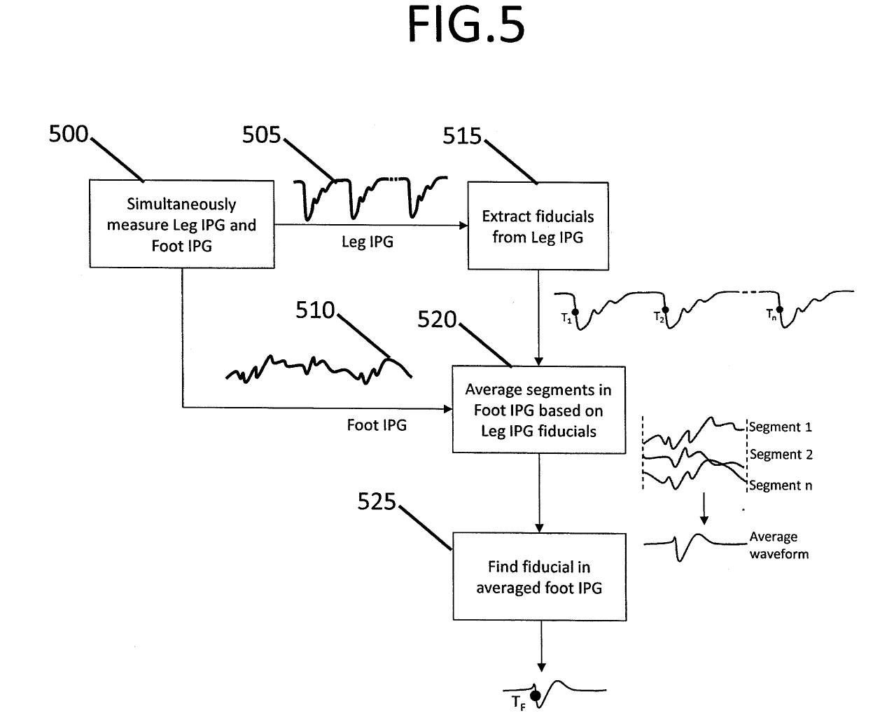

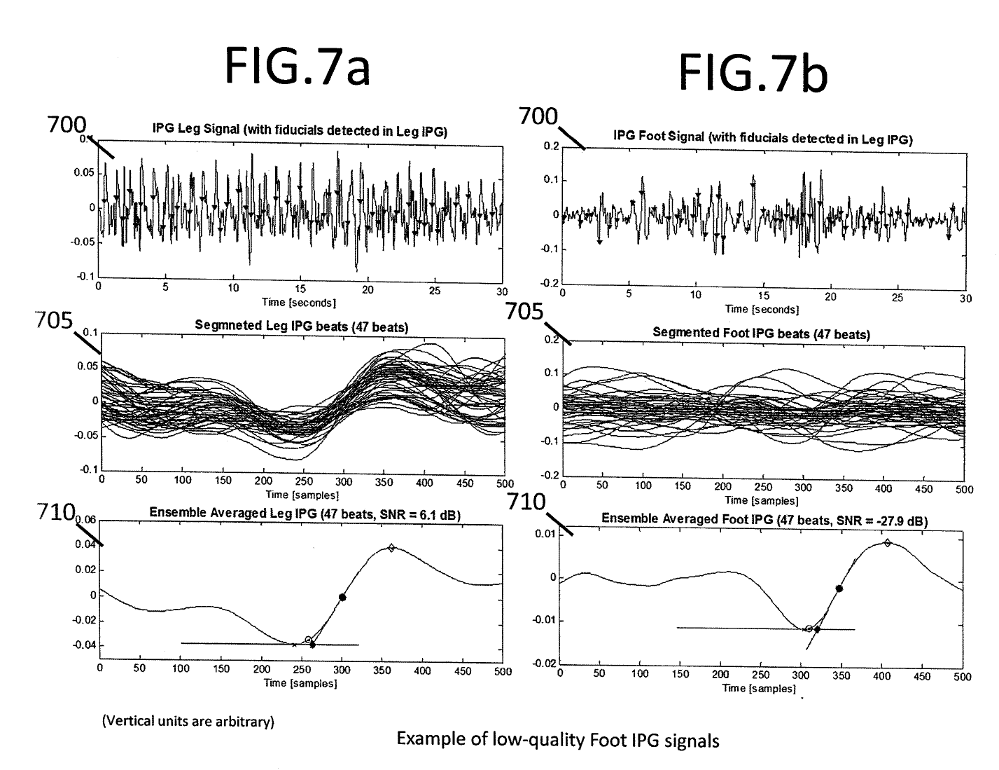

FIG. 5 shows an example flowchart depicting signal processing to segment individual Foot IPG “beats” to produce an averaged IPG waveform of improved SNR, which is subsequently used to determine the fiducial of the averaged Foot IPG, consistent with various aspects of the present disclosure. Similar to the method shown in FIG. 4, the Leg IP and the Foot IPG are simultaneously measured (500), the Leg IPG is low-pass filtered (505), the foot IPG is low-pass filtered (510), and segments starting from the timings extracted (515) from the Leg IPG (reference timings). The segments of the Foot IPG extracted based on the Leg IPG timings are ensemble-averaged (520) to produce a higher SNR Foot IPG pulse. From this ensemble-averaged signal, the start of the pulse is extracted using the same intersecting tangent approach as described earlier. This approach enables the extraction of accurate timings in the Foot IPG even if the impedance signal is dominated by noise, as shown in FIG. 7b. These timings are used together with timings extracted from the BCG for the purpose of computing the PTT and (PWV). Timings derived from ensemble-averaged waveforms and individual waveforms can also be both extracted, for the purpose of comparison, averaging and error-detection.

Specific timings extracted from the IPG pulses (from either leg or foot) are related (but not limited) to the peak of the pulse, the minimum preceding the peak, or the maximum second derivative (maximum rate of acceleration) preceding the point of maximum derivative. An IPG pulse and the extraction of a fiducial (525) in the IPG can be performed by other signal processing methods, including (but not limited to) template matching, cross-correlation, wavelet-decomposition, or short window Fourier transform.

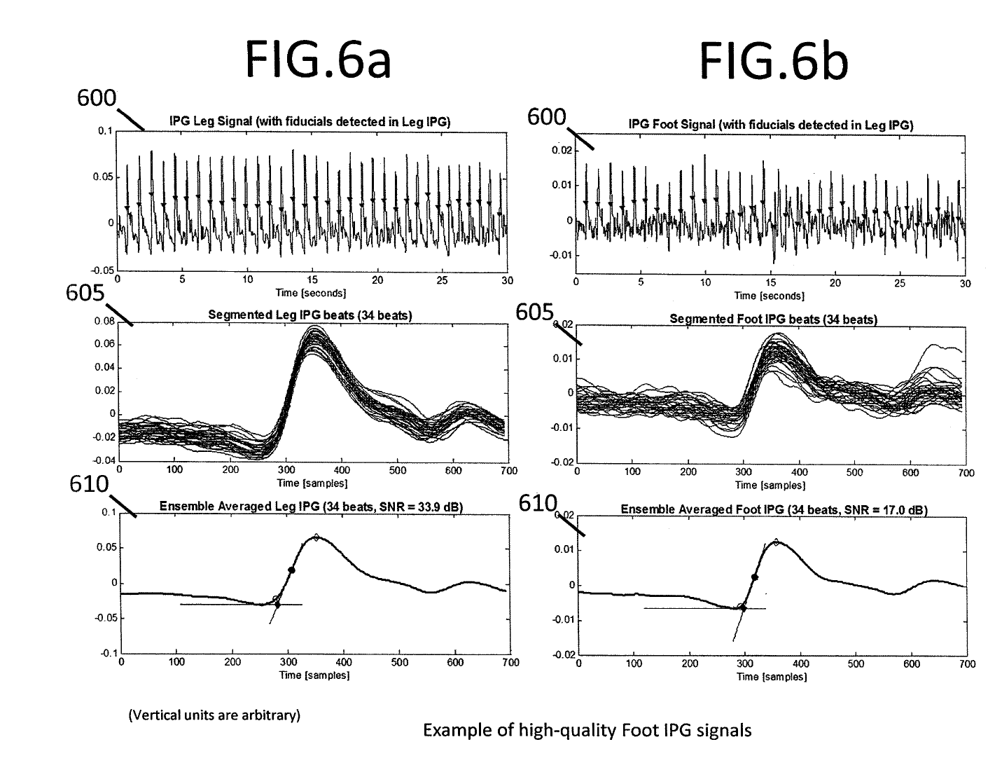

FIG. 6a shows examples of the Leg IPG signal with fiducials (plot 600); the segmented Leg IPG into beats (plot 605); and the ensemble-averaged Leg IPG beat with fiducials and calculated SNR (plot 610), for an exemplary high-quality recording, consistent with various aspects of the present disclosure. FIG. 6b shows examples of the Foot IPG signal with fiducials derived from the Leg IPG fiducials (plot 600); the segmented Foot IPG into beats (plot 605); and the ensemble-averaged Foot IPG beat with fiducials and calculated SNR (plot 610), for an exemplary high-quality recording, consistent with various aspects of the present disclosure.

FIG. 7a shows examples of the Leg IPG signal with fiducials (plot 700); the segmented Leg IPG into beats (plot 705); and the ensemble averaged Leg IPG beat with fiducials and calculated SNR (plot 710), for an exemplary low-quality recording, consistent with various aspects of the present disclosure.

FIG. 7b shows examples of the Foot IPG signal with fiducials derived from the Leg IPG fiducials (plot 700); the segmented Foot IPG into beats (plot 705); and the ensemble-averaged Foot IPG beat with fiducials and calculated SNR (plot 710), for an exemplary low-quality recording, consistent with aspects of the present disclosure.

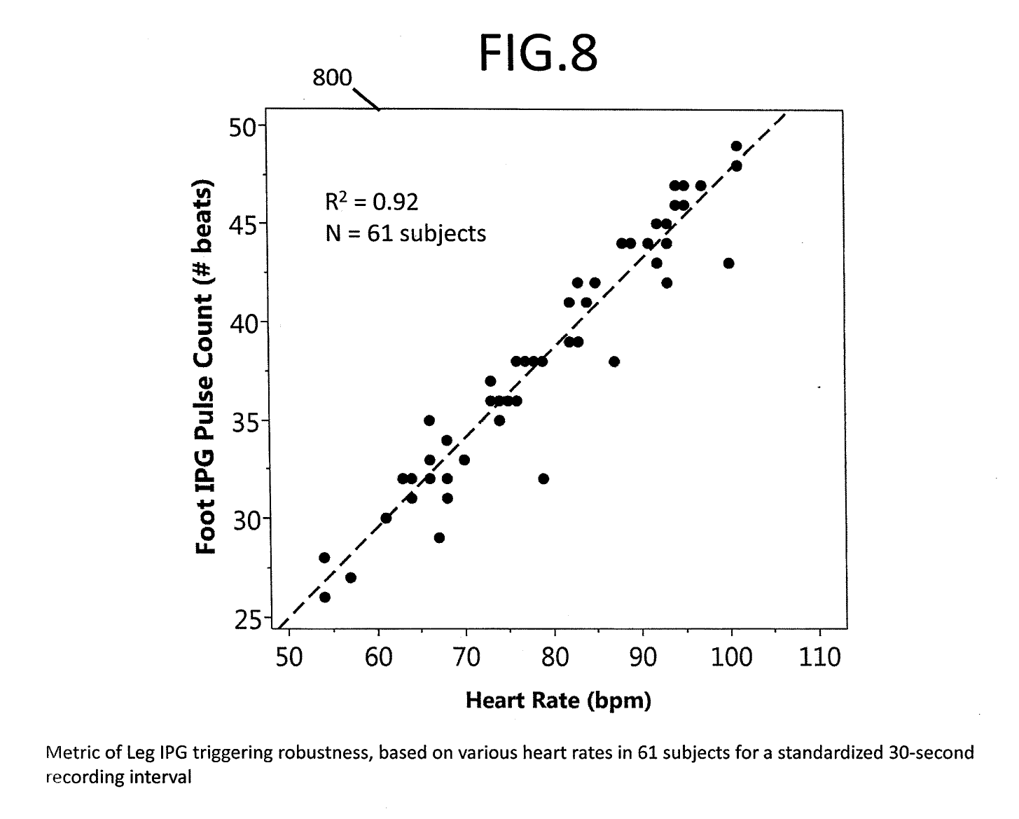

FIG. 8 shows an example correlation plot 800 for the reliability in obtaining the low SNR Foot IPG pulse for a 30-second recording, using the first impedance signal as the trigger pulse, from a study including 61 test subjects with various heart rates, consistent with various aspects of the present disclosure.

In certain embodiments, a dual-Foot IPG is measured, allowing the detection of blood pressure pulses in both feet. Such information can be used for diagnostic of peripheral arterial diseases (PAD) by comparing the relative PATs in both feet to look for asymmetries. It can also increase the robustness of the measurement by allowing one foot to have poor contact with electrodes (or no contact at all). SNR measurements can be used to assess the quality of the signal in each foot, and to select the best one for downstream analysis. Timings extracted from each foot can be compared and set to flag potentially inaccurate PWV measurements due to arterial peripheral disease, in the event these timings are different by more than a threshold. Alternatively, timings from both feet are pooled to increase the overall SNR if their difference is below the threshold.

In certain embodiments, the disclosure is used to measure a PWV, where the IPG is augmented by the addition of BCG sensing into the weighing scale to determine characteristic fiducials between the BCG and Leg IPG trigger, or the BCG and Foot IPG. The BCG sensors are comprised typically of the same strain gage set used to determine the bodyweight of the user. The load cells are typically wired into a bridge configuration to create a sensitive resistance change with small displacements due to the ejection of the blood into the aorta, where the circulatory or cardiovascular force produce movements within the body on the nominal order of 1-3 Newtons. BCG forces can be greater than or less than the nominal range in cases such as high or low cardiac output.

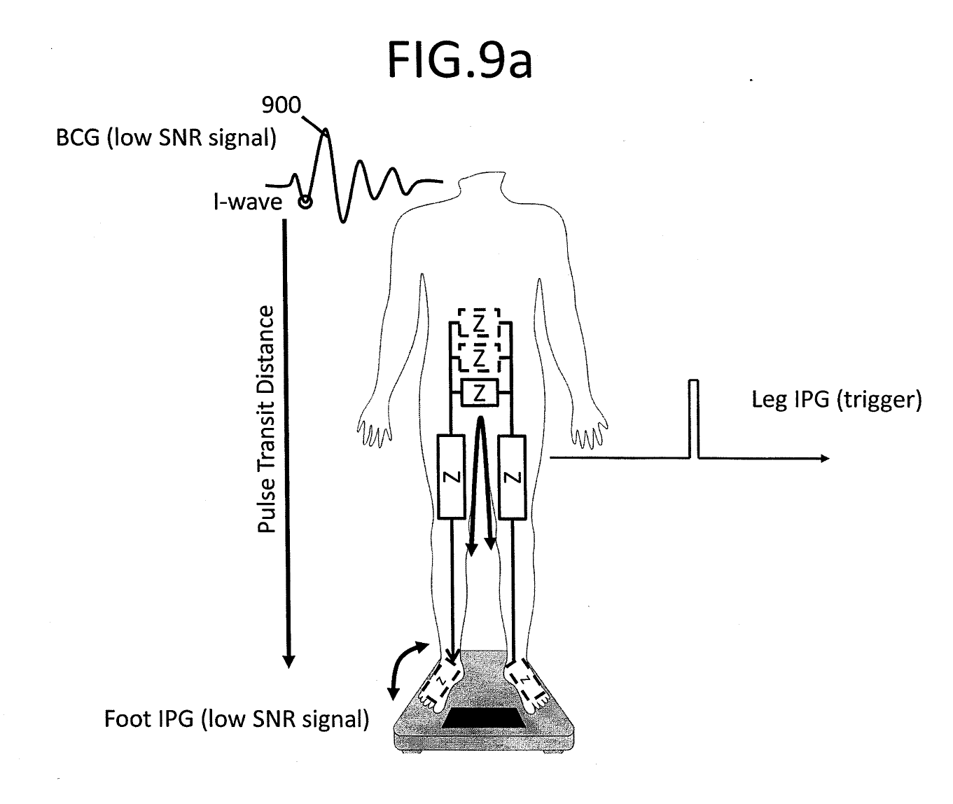

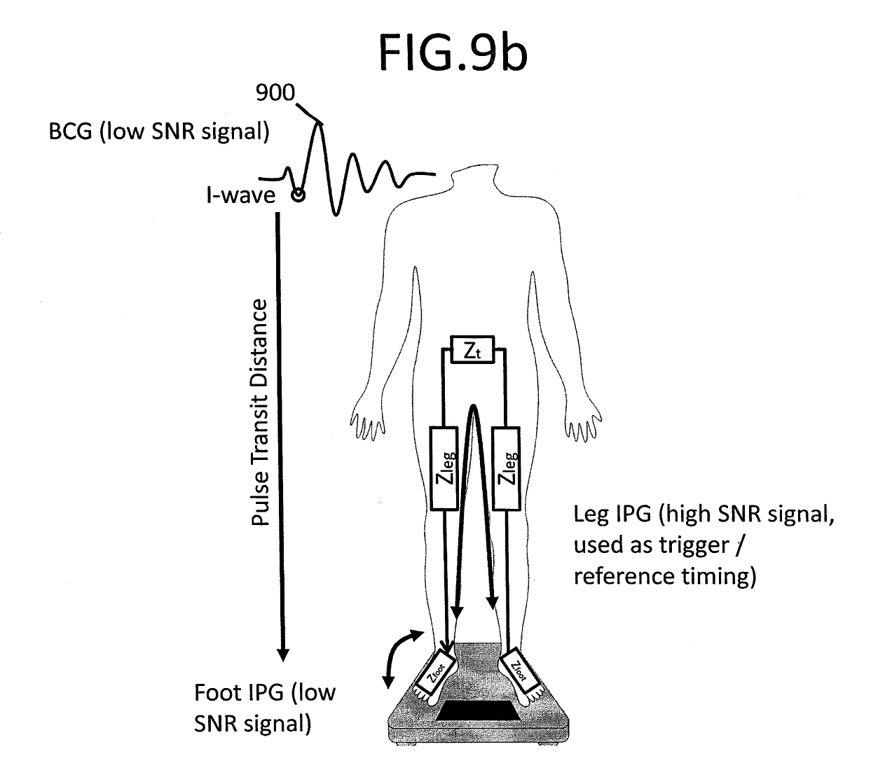

FIGS. 9a-b show example configurations to obtain the PTT, using the first IPG as the triggering pulse for the Foot IPG and BCG, consistent with various aspects of the present disclosure. The I-wave of the BCG 900 normally depicts the headward force due to cardiac ejection of blood into the ascending aorta which is used as a timing fiducial indicative of the pressure pulse initiation of the user's proximal aorta relative to the user's heart. The J-wave is indicative of timings in the systole phase and also incorporates information related to the strength of cardiac ejection and the ejection duration. The K-Wave provides systolic and vascular information of the user's aorta. The characteristic timings of these and other BCG waves are used as fiducials that can be related to fiducials of the IPG signals of the present disclosure.

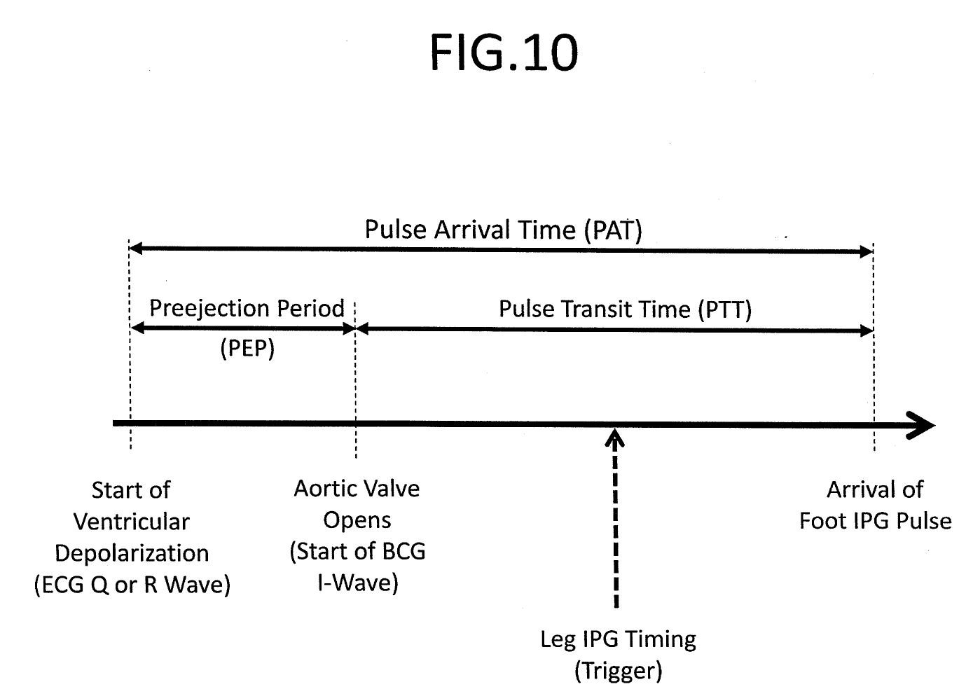

FIG. 10 shows nomenclature and relationships of various cardiovascular timings, consistent with various aspects of the present disclosure.

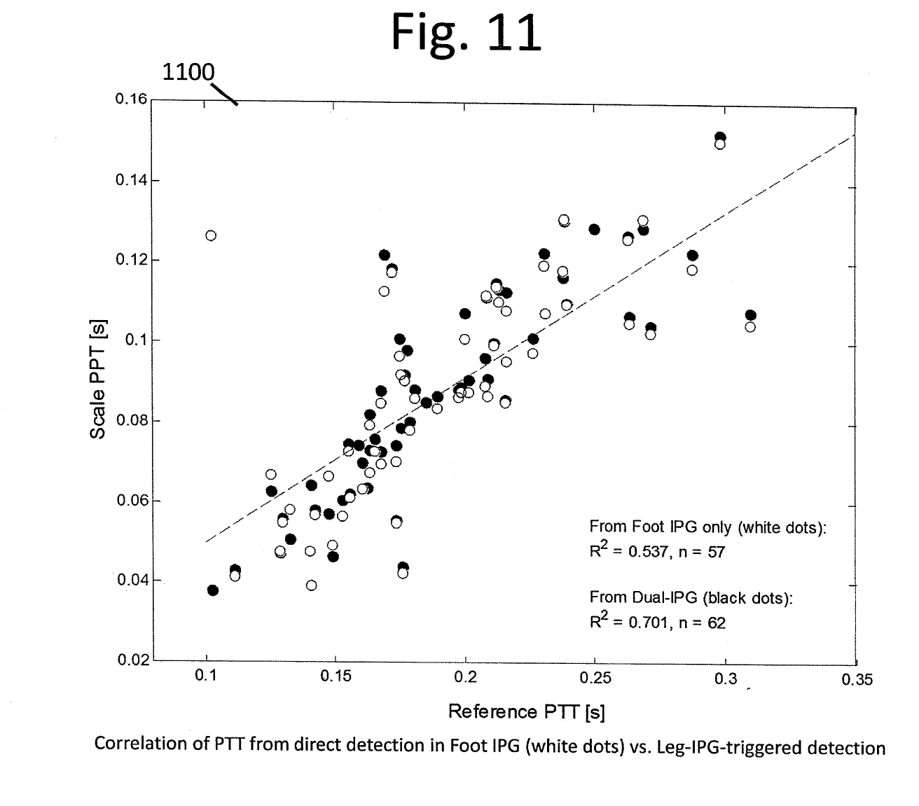

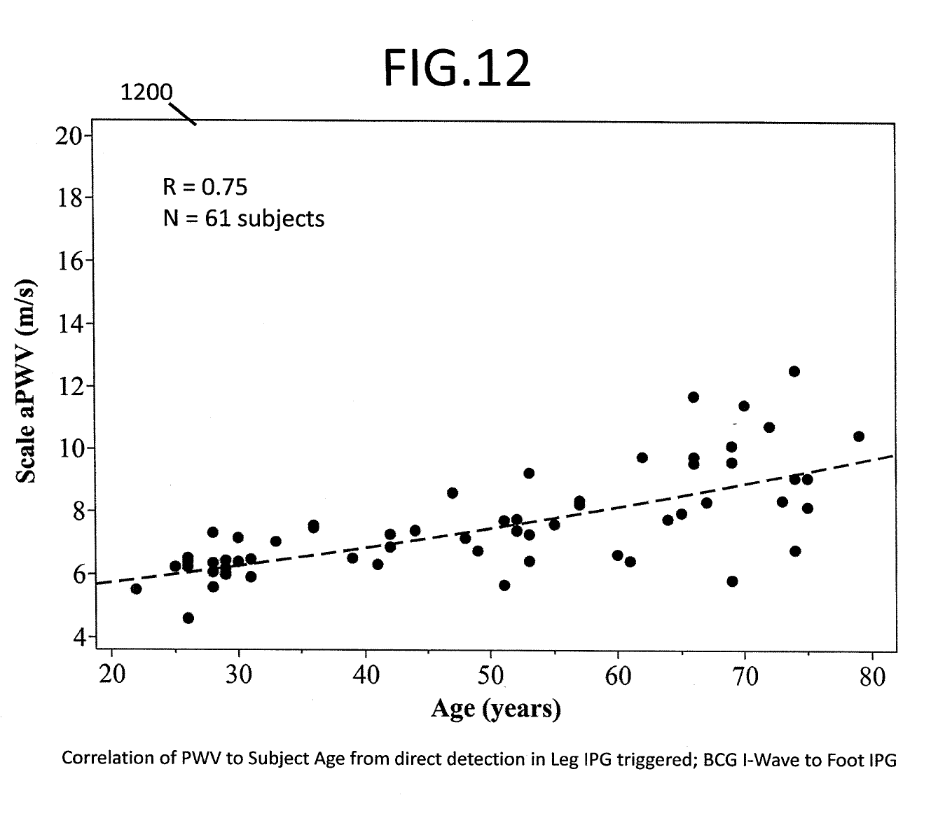

FIG. 11 shows an example graph 1100 of PTT correlations for two detection methods (white dots) Foot IPG only, and (black dots) Dual-IPG method; and FIG. 12 shows an example graph 1200 of PWV obtained from the present disclosure compared to the ages of 61 human test subjects, consistent with various aspects of the present disclosure.

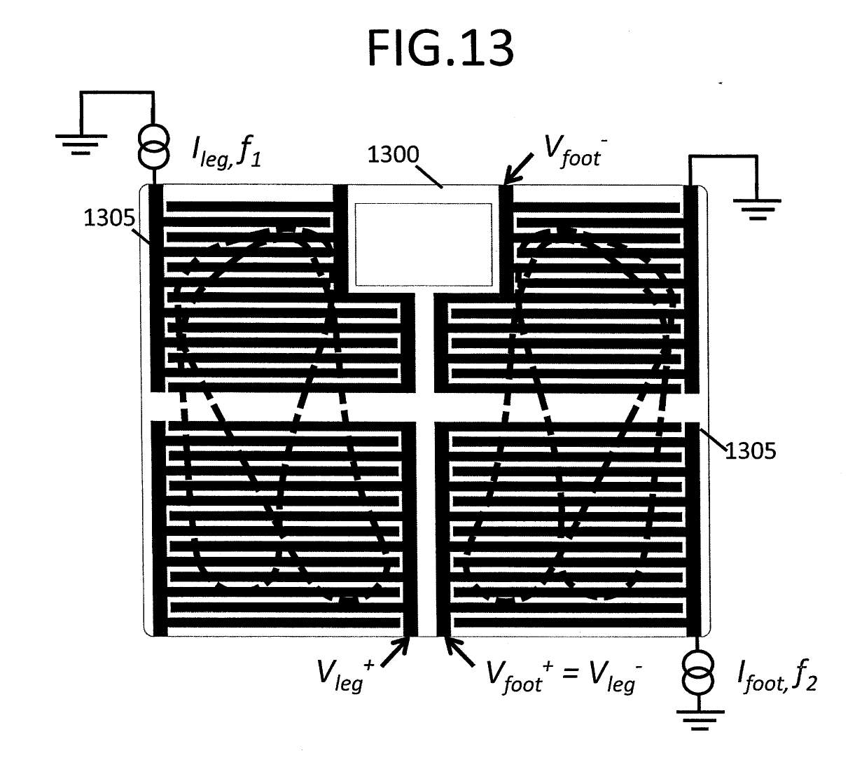

FIG. 13 shows an example of a scale 1300 with integrated foot electrodes 1305 to inject and sense current from one foot to another foot, and within one foot.

FIG. 14a-c shows various examples of a scale 1400 with interleaved foot electrodes 1405 to inject/sense current from one foot to another foot, and measure Foot IPG signals in both feet.

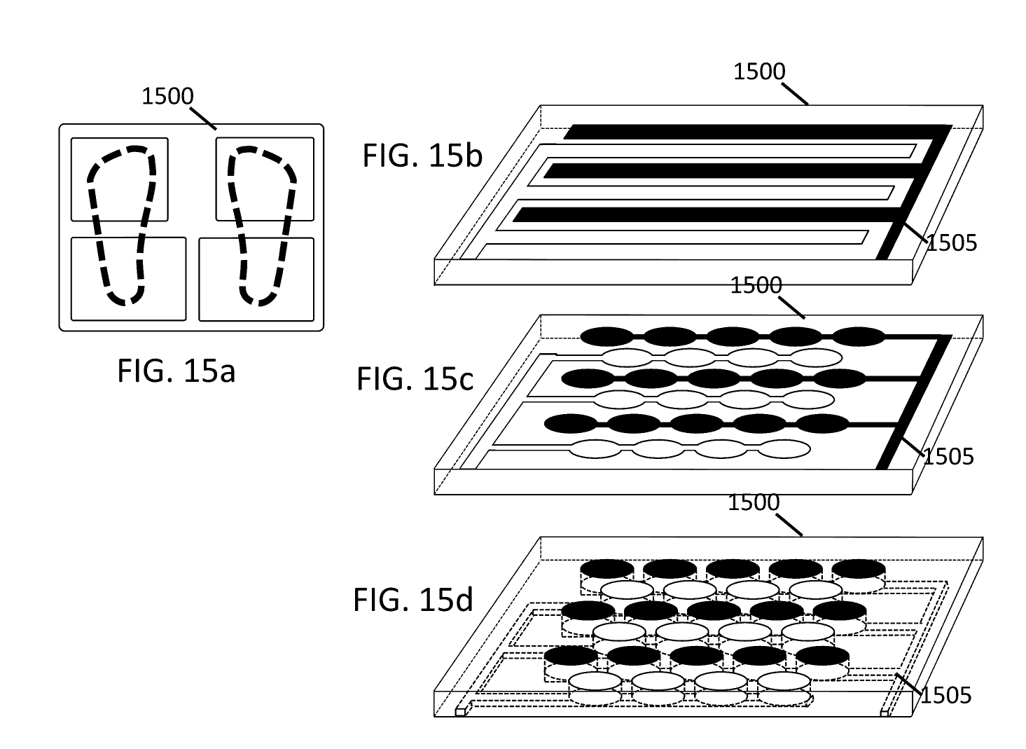

FIGS. 15a-d shows an example breakdown of a scale 1500 with interleaved foot electrodes 1505 to inject and sense current from one foot to another foot, and within one foot.

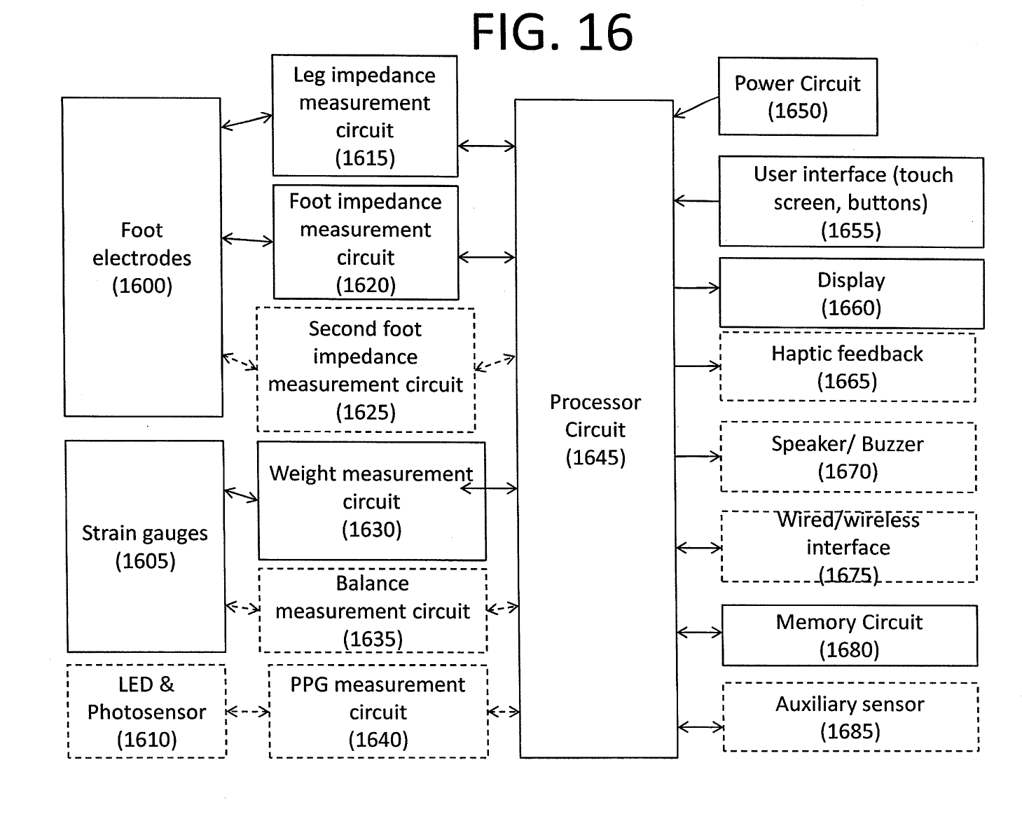

FIG. 16 shows an example block diagram of circuit-based building blocks, consistent with various aspects of the present disclosure. The various circuit-based building blocks shown in FIG. 16 can be implemented in connection with the various aspects discussed herein. In the example shown, the block diagram includes foot electrodes 1600 that can collect the IPG signals. Further, the block diagram includes strain gauges 1605, and an LED/photosensor 1610. The foot electrodes 1600 is configured with a leg impedance measurement circuit 1615, a foot impedance measurement circuit 1620, and an optional second foot impedance measurement circuit 1625. The leg impedance measurement circuit 1615, the foot impedance measurement circuit 1620, and the optional second foot impedance measurement circuit 1625 report the measurements collected to a processor circuitry 1645.

The processor circuitry 1645 collects data from a weight measurement circuit 1630 and an optional balance measurement circuit 1635 that are configured with the strain gauges 1605. Further, an optional photoplethysmogram (PPG) measurement circuit 1640, which collects data from the LED/photosensor 1610, provides data to the processor circuitry 1645.

The processor circuitry 1645 is powered via a power circuit 1650. Further, the processor circuitry 1645 collects user input data from a user interface 1655 (e.g., iPad®, smart phone and/or other remote user handy/CPU with a touch screen and/or buttons). The data collected/measured by the processor circuitry 1645 is shown to the user via a display 1660. Additionally, the data collected/measured by the processor circuitry 1645 can be stored in a memory circuit 1680. Further, the processor circuitry 1645 can optionally control a haptic feedback circuit 1665, a speaker or buzzer 1670, a wired/wireless interface 1675, and an auxiliary sensor 1685 for one-way or two-way communication between the scale and the user.

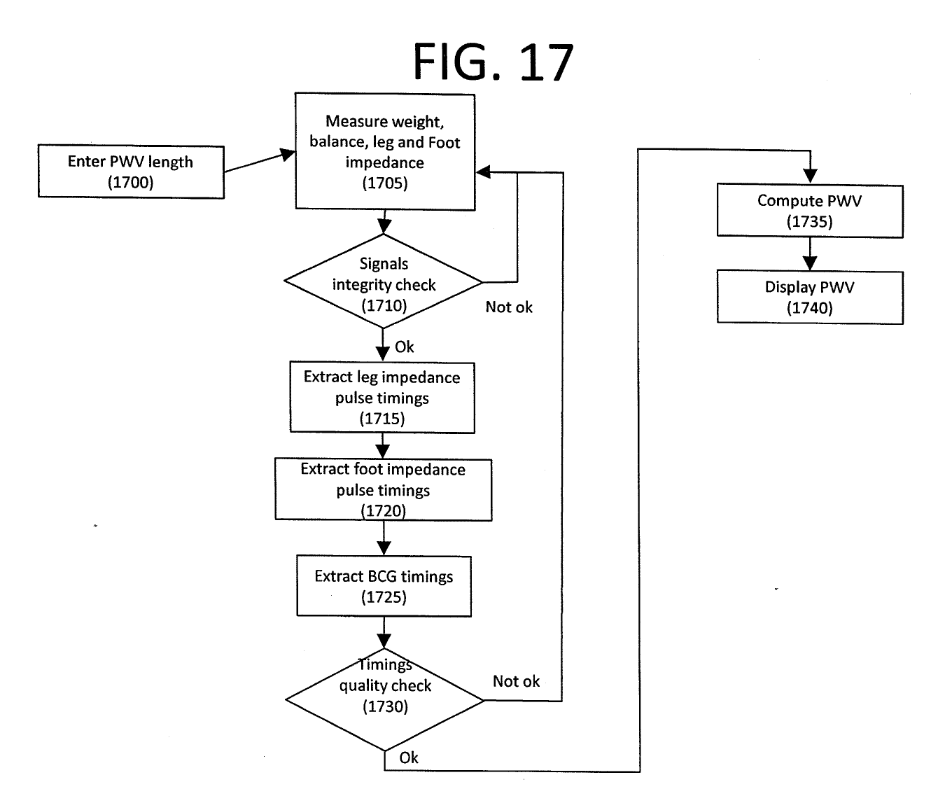

FIG. 17 shows an example flow diagram, consistent with various aspects of the present disclosure. At block 1700, a PWV length is entered. At block 1705, a user's weight, balance, leg, and foot impedance are measured. At 1710, the integrity of signals is checked (e.g., SNR). If the signal integrity check is not met, the user's weight, balance, leg, and foot impedance are measured again (block 1705), if the signals integrity check is met, the leg impedance pulse timings are extracted (as is shown at block 1715). At block 1720, foot impedance and pulse timings are extracted, and at block 1725, BCG timings are extracted. At block 1730, a timings quality check is performed. If the timings quality check is not validated, the user's weight, balance, leg and foot impedance are again measured (block 1705). If the timings quality check is validated, the PWV is calculated (as is shown at block 1735). At block 1740, the PWV is displayed to the user.

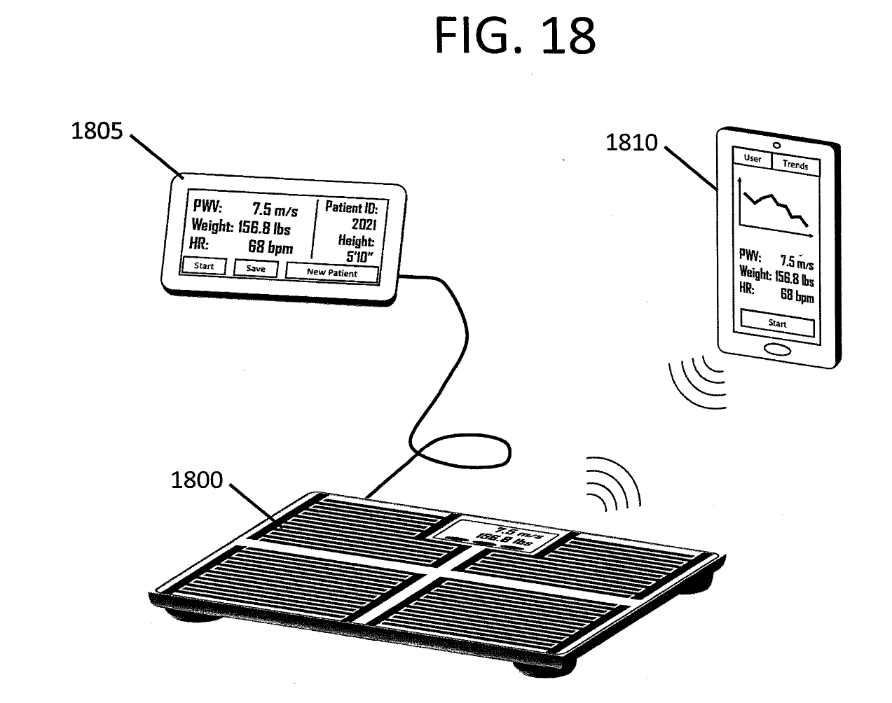

FIG. 18 shows an example scale 1800 communicatively coupled to a wireless device, consistent with various aspects of the present disclosure. As described herein, a display 1805 displays the various aspects measured by the scale 1800. The scale, in some embodiments, also wirelessly broadcast the measurements to a wireless device 1810. The wireless device 1810, in various embodiments, is implemented as an iPad®, smart phone or other CPU to provide input data for configuring and operating the scale.

As an alternative or complementary user interface, the scale includes a FUI which can be enabled/implementable by one or more foot-based biometrics (for example, with the user being correlated to previously-entered user weight, and/or foot size/shape). The user foot-based biometric, in some embodiments, is implemented by the user manually entering data (e.g., a password) on the upper surface or display area of the scale. In implementations in which the scale is configured with a haptic, capacitive or flexible pressure-sensing upper surface, the (upper surface/tapping) touching from or by the user is sensed in the region of the surface and processed according to conventional X-Y grid Signal processing in the logic circuitry/CPU that is within the scale. By using one or more of the accelerometers located within the scale at its corners, such user data entry is sensed by each such accelerometer so long as the user's toe, heel or foot pressure associated with each tap provides sufficient force. Although the present discussion refers to a FUI, embodiments are not so limited. Various embodiments include internal or external GUIs that are in communication with the scale and used to obtain a biometric and that can be in place of the FUI and/or in combination with a FUI. For example, a user device having a GUI, such as tablet, is in communication with the scale via a wired or wireless connection. The user device obtains a biometric, such a finger print, and communicates the biometric to the scale.

In various embodiments, the above discussed user-interface is used with other features described herein for the purpose of storing and securing user sensitive data such as: the configuration data input by the user, the biometric and/or passwords entered by the user, and the user-specific health related data which might include less sensitive data (e.g., the user's weight) and more sensitive data (e.g., the user's scale obtains cardiograms and other data generated by or provided to the scale and associated with the user's symptoms and/or diagnoses). For such user data, the above described biometrics are used as directed by the user for indicating and defining protocol to permit such data to be exported from the scale to other remote devices indoor locations. In more specific embodiments, the scale operates in different modes of data security including, for example: a default mode in which the user's body mass and/or weight is displayed regardless of any biometric which would associate with the specific user standing on the scale; another mode in which complicated data (or data reviewed infrequently) is only exported from the scale under specific manual commands provided to the scale under specific protocols; and another mode or modes in which the user-specific data that is collected from the scale is processed and accessed based on the type of data. Such data categories include categories of different level of importance and/or sensitivities such as the above-discussed high and low level data and other data that might be very specific to a symptom and/or degrees of likelihood for diagnoses. Optionally, the CPU in the scale is also configured to provide encryption of various levels of the user's sensitive data.

For example, in accordance with various embodiments, the above-described FUI is used to provide portions of the clinical indications (e.g., scale-obtained physiological data) and/or additional health information to the user. In some embodiments, the scale includes a display configuration filter (e.g., circuitry and/or computer readable medium) configured to discern the data to display to the user and display portion. The display configuration filter discerns which portions of the clinical indications and/or additional health information to display to the user on the FUI based on various user demographic information (e.g., age, gender, height, diagnosis) and the amount of data. For example, the clinical indication may include an amount of data that if all the data is displayed on the FUI, the data is difficult for a person to read and/or uses multiple display screens.

The display configuration filter discerns portions of the data to display using the scale user interface, such as synopsis of the clinical indication (or additional health information) and an indication that additional data is displayed on another user device, and other portions to display on the other user device. The other user device is selected by the scale (e.g., the filter) based on various communications settings. The communication settings include settings such as user settings (e.g., the user identifying user devices to output data to), scale-based biometrics (e.g., user configures scale, or default settings, to output data to user devices in response to identifying scale-based biometrics), and/or proximity of the user device (e.g., the scale outputs data to the closest user device among a plurality of user devices and/or in response to the user device being within a threshold distance from the scale), among other settings. For example, the scale determines which portions of the clinical indication or additional health information to output and outputs the remaining portion of the clinical indication or additional health information to a particular user device based on user settings/communication authorization (e.g., what user devices are authorized by the user to receive particular user data from the scale), and proximity of the user device to the scale. The determination of which portions to output is based on what type of data is being displayed, how much data is available, and the various user demographic information (e.g., an eighteen year old is able to see better than a fifty year old).

For example, in some specific embodiments, the scale operates in different modes of data security and communication. The different modes of data security and communication are enabled in response to biometrics identified by the user and using the foot-controlled user interface. In some embodiments, the scale is used by multiple users and/or the scale operates in different modes of data security and communication in response to identifying the user and based on biometrics. The different modes of data security and communication include, for example: a first mode (e.g., default mode) in which the user's body mass and/or weight is displayed regardless of any biometric which would associate with the specific user standing on the scale and no data is communicated to external circuitry; a second mode in which complicated/more-sensitive data (or data reviewed infrequently) is only exported from the scale under specific manual commands provided to the scale under specific protocols and in response to a biometric; and third mode or modes in which the user-specific data that is collected from the scale is processed and accessed based on the type of data and in response to a biometric. Such data categories include categories of different levels of importance and/or sensitivities such as the above-discussed high and low level data and other data that might be very specific to a symptom and/or degrees of likelihood for diagnoses. Optionally, the CPU in the scale is also configured to provide encryption of various levels of the user's sensitive data.

In some embodiments, the different modes of data security and communication are enabled in response to recognizing the user standing on the scale using a biometric and operating in a particular mode of data security and communication based on user preferences and/or services activated. For example, the different modes of operation include the default mode (as discussed above) in which certain data (e.g., categories of interest, categories of user-sensitive user data, or historical user data) is not communicated from the scale to external circuitry, a first communication mode in which data is communicated to external circuitry as identified in a user profile, a second or more communication modes in which data is communicated to a different external circuitry for further processing. The different communication modes are enabled based on biometrics identified from the user and user settings in a user profile corresponding with each user.

In a specific embodiment, a first user of the scale may not be identified and/or have a user profile set up. In response to the first user standing on the scale, the scale operates in a default mode. During the default mode, the scale displays the user's body mass and/or weight on the user display and does not output user data. A second user of the scale has a user profile set up that indicates the user would like data communicated to a computing device of the user. When the second user stands on the scale, the scale recognizes the second user based on a biometric and operates in a first communication mode. During the first communication mode, the scale outputs at least a portion of the user data to an identified external circuitry. For example, the first communication mode allows the user to upload data from the scale to a user identified external circuitry (e.g., the computing device of the user). The information may include additional health information and/or user information that has low-user sensitivity. In the first communication mode, the scale performs the processing of the raw sensor data and/or the external circuitry can. For example, the scale sends the raw sensor data and/or additional health information to a user device of the user. The computing device may not provide access to the raw sensor data to the user and/or can send the raw sensor data to another external circuitry for further processing in response to a user input. For example, the computing device can ask the user if the user would like additional health information and/or regulated health information as a service. In response to receiving an indication the user would like the additional health information and/or regulated health information, the computing device outputs the raw sensor data and/or non-regulated health information to another external circuitry for processing, providing to a physician for review, and controlling access, as discussed above.

In one or more additional communication modes, the scale outputs raw sensor data to an external circuitry for further processing. For example, during a second communication mode and a third communication, the scale sends the raw sensor data and other data to external circuitry for processing, such as to a remote user-physiological device for correlation and processing. Using the above-provided example, a third user of the scale has a user profile set up that indicates the third user would like scale-obtained data to be communicated to a remote user-physiological device for further processing, such as to correlate the cardio-data sets and/or further process the correlated data sets. When the third user stands on the scale, the scale recognizes the third user based on one or more biometrics and operates in a second communication mode. During the second communication mode, the scale outputs the raw sensor data to the remote user-physiological device. The remote user-physiological device correlates the raw sensor data from the scale with cardio-physiological data from the remote user-physiological device, determines at least one physiological parameter of the user, and, optionally, derives additional health information. In some embodiments, the remote user-physiological device outputs data, such as the physiological parameter or additional health information to the scale. The scale, in some embodiments, displays a synopsis of the additional health information and outputs a full version of the additional health information to another user device for display (such as, using the filter described above) and/or an indication that additional health information can be accessed.

A fourth user of the scale has a user profile set up that indicates the fourth user has enabled a service to access regulated health information. When the fourth user stands on the scale, the scale recognizes the user based on one or more biometrics and operates in a fourth communication mode. In the fourth communication mode, the scale outputs raw sensor data to the external circuitry, and the external circuitry processes the raw sensor data and controls access to the data. For example, the external circuitry may not allow access to the regulated health information until a physician reviews the information. In some embodiments, the external circuitry outputs data to the scale, in response to physician review. For example, the output data can include the regulated health information and/or an indication that regulated health information is ready for review. The external circuitry may be accessed by the user, using the scale and/or another user device. In some embodiments, using the FUI of the scale, the scale displays the regulated health information to the user. The scale, in some embodiments, displays a synopsis of the regulated health information (e.g., clinical indication) and outputs the full version of regulated health information to another user device for display (such as, using the filter described above) and/or an indication that the regulated health information can be accessed to the scale to display. In various embodiments, if the scale is unable to identify a particular (high security) biometric that enables the fourth communication mode, the scale may operate in a different communication mode and may still recognize the user. For example, the scale may operate in a default communication mode in which the user data collected by the scale is stored in a user profile corresponding to the fourth user and on the scale. In some related embodiments, the user data is output to the external circuitry at a different time.

Although the present embodiments illustrates a number of security and communication modes, embodiments in accordance with the present disclosure can include additional or fewer modes. Furthermore, embodiments are not limited to different modes based on different users. For example, a single user may enable different communication modes in response to particular biometrics of the user identified and/or based on user settings in a user profile.

In various embodiments, the scale defines a user data table that defines types of user data and sensitivity values of each type of user data. In specific embodiments, the FUI displays the user data table. In other specific embodiments a user interface of a smartphone, tablet, and/or other computing device displays the user data table. For example, a wired or wireless tablet is used, in some embodiments, to display the user data table. The sensitivity values of each type of user data, in some embodiments, define in which communication mode(s) the data type is communicated and/or which biometric is used to enable communication of the data type. In some embodiments, a default or pre-set user data table is displayed and the user revises the user data table using the FUI. The revisions are in response to user inputs using the user's foot and/or contacting or moving relative to the FUI. Although the embodiments are not so limited, the above (and below) described control and display is provided using a wireless or wired tablet or other computing device as a user interface. The output to the wireless or wired tablet, as well as additional external circuitry, is enabled using biometrics. For example, the user is encouraged, in particular embodiments, to configure the scale with various biometrics. The biometric include scale-based biometrics and biometrics from the tablet or other user computing device. The biometric, in some embodiments, used to enable output of data to the tablet and/or other external circuitry includes a higher integrity biometric (e.g., higher likelihood of identifying the user accurately) than a biometric used to identify the user and stored data on the scale.

An example user data table is illustrated below:

| | User-data Type | | | | |
|---|---|---|---|---|---|
| | | Body Mass | User- | Physician- | Scale-stored |
| | Weight, | Index, user | Specific | Provided | suggestions |
| | local | specific | Advertise- | Diagnosis/ | (symptoms & |
| | weather | news | ments | Reports | diagnosis) |
| Sensi- | 1 | 3 | 5 | 10 | 9 |
| tivity | | | | | |
| (10 = | | | | | |
| highest, | | | | | |
| 1 = | | | | | |
| lowest) | | | | | |

The above-displayed table is for illustrative purposes and embodiments in accordance with the present disclosure can include additional user-data types than illustrated, such as cardiogram characteristics, clinical indications, physiological parameters, user goals, demographic information, etc. In various embodiments, the user data table includes additional rows than illustrated. The rows, in specific embodiments, include different data input sources and/or sub-data types (as discussed below). Data input sources include source of the data, such as physician provided, input from the Internet, user provided, from the external circuitry. The different data from the data input sources, in some embodiments, is used alone or in combination.

In various embodiments, the user adjusts the table displayed above to revise the sensitivity values of each data type. Further, although the above-illustrated table includes a single sensitivity value for each data type, in various embodiments, one or more of the data types are separated into sub-data types and each sub-data type has a sensitivity value. As an example, the user-specific advertisement is separated into: prescription advertisement, external device advertisements, exercise advertisements, and diet plan advertisement. Alternatively and/or in addition, the sub-data types for user-specific advertisement include generic advertisements based on a demographic of the user and advertisements in response to scale collected data (e.g., advertisement for a device in response to physiologic parameters), as discussed further herein.

For example, weight data includes the user's weight and historical weight as collected by the scale. In some embodiments, weight data includes historical trends of the user's weight and correlates to dietary information and/or exercise information, among other user data. Body mass index data, includes the user's body mass index as determined using the user's weight collected by the scale and height. In some embodiments, similar to weight, body mass index data includes history trends of the user's body mass index and correlates to various other user data.

User-specific advertisement data includes various prescriptions, exercise plans, dietary plans, and/or other user devices and/or sensors for purchase, among other advertisements. The user-specific advertisements, in various embodiments, are correlated to input user data and/or scale-obtained data. For example, the advertisements include generic advertisements that are relevant to the user based on a demographic of the user. Further, the advertisements include advertisements that are responsive to scale collected data (e.g., physiological parameter includes a symptom or problem and advertisement is correlated to the symptom or problem). A number of specific examples include advertisements for beta blockers to slow heart rate, advertisements for a user wearable device (e.g., Fitbit®) to monitor heart rate, and advertisements for a marathon exercise program (such as in response to an indication the user is training for a marathon), etc.

Physician provided diagnosis/report data includes data provided by a physician and, in various embodiments, is in responsive to the physician reviewing the scale-obtained data. For example, the physician provided diagnosis/report data includes diagnosis of a disorder/condition by a physician, prescription medication prescribed by a physician, and/or reports of progress by a physician, among other data. In various embodiments, the physician provided diagnosis/reports are provided to the scale from external circuitry, which includes and/or accesses a medical profile of the user.

Scaled stored suggestion data includes data that provides suggestions or advice for symptoms, diagnosis, and/or user goals. For example, the suggestions include advice for training that is user specific (e.g., exercise program based on user age, weight, and cardiogram data or exercise program for training for an event or reducing time to complete an event, such as a marathon), suggestions for reducing symptoms including dietary, exercise, and sleep advice, and/or suggestions to see a physician, among other suggestions. Further, the suggestions or advice include reminders regarding prescriptions. For example, based on physician provided diagnosis/report data and/or user inputs, the scale identifies the user is taking a prescription medication. The identification includes the amount and timing of when the user takes the medication, in some embodiments. The scale reminds the user and/or asks for verification of consumption of the prescription medication using the FUI.

As further specific examples, recent discoveries may align and associate different attributes of scale-based user data collected by the scale to different tools, advertisements, and physician provided diagnosis. For example, it has recently been discovered that atrial fibrillation is more directly correlated with obesity. The scale collects various user data and monitors weight and various components/symptoms of atrial fibrillation. In a specific embodiment, the scale recommends/suggests to the user to: closely monitor weight, recommends a diet, goals for losing weight, and correlates weight gain and losses for movement in cardiogram data relative to arrhythmia. The movement in cardiogram data relative to arrhythmia, in specific embodiments, is related to atrial fibrillation. For example, atrial fibrillation is associated with indiscernible p-waves and beat to beat fluctuations. Thereby, the scale correlates weight gain/loss with changes in amplitude (e.g., discernibility) of a p-wave of a cardiogram (preceding a QRS complex) and changes in beat to beat fluctuations.

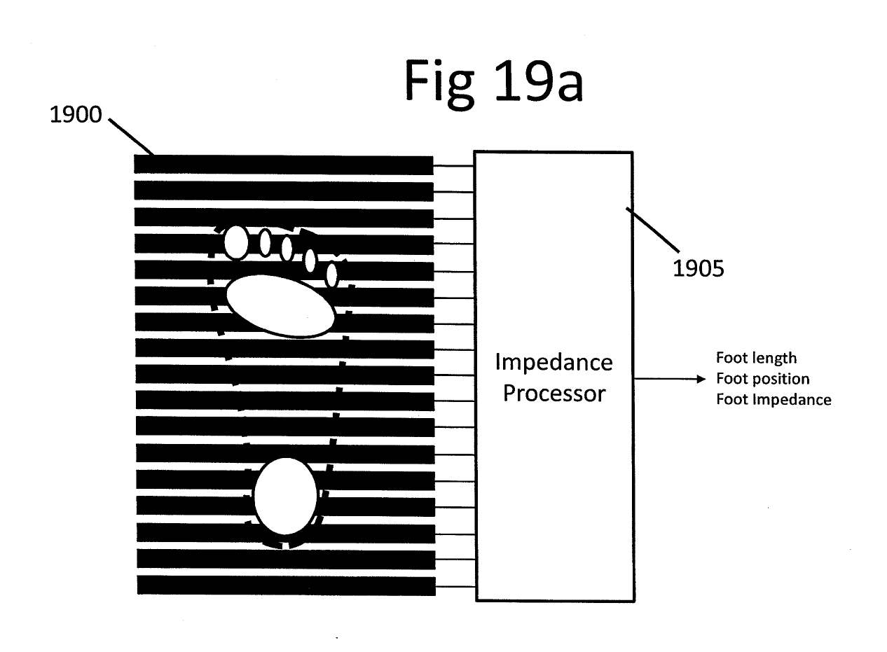

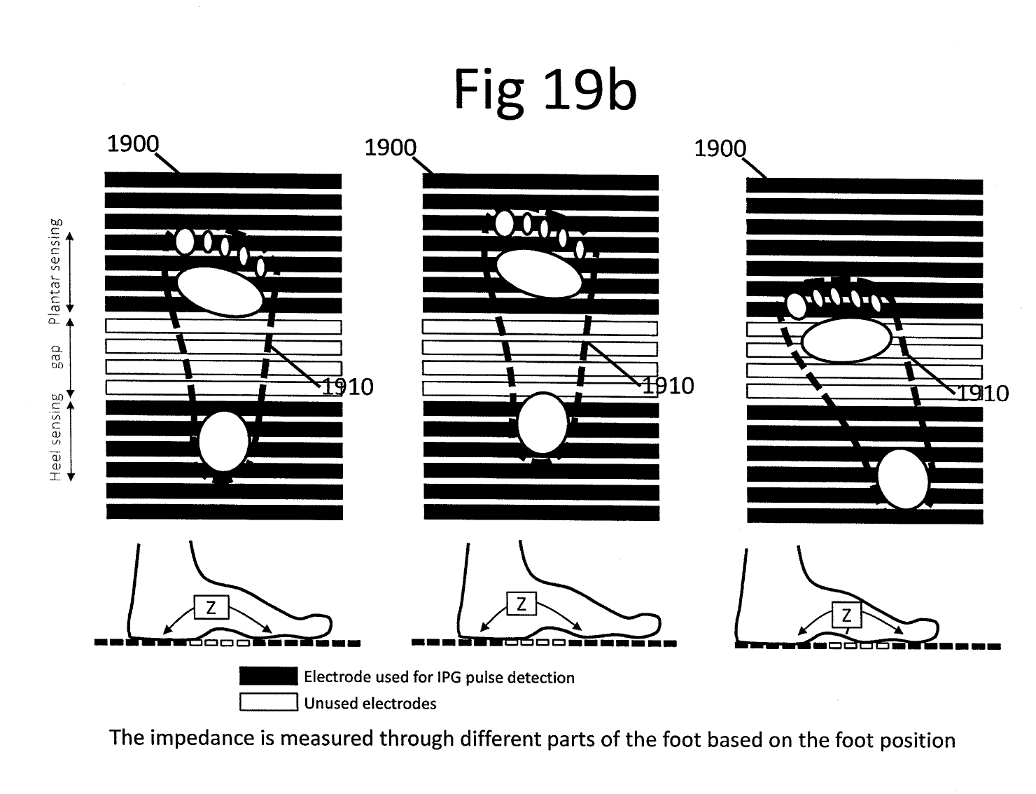

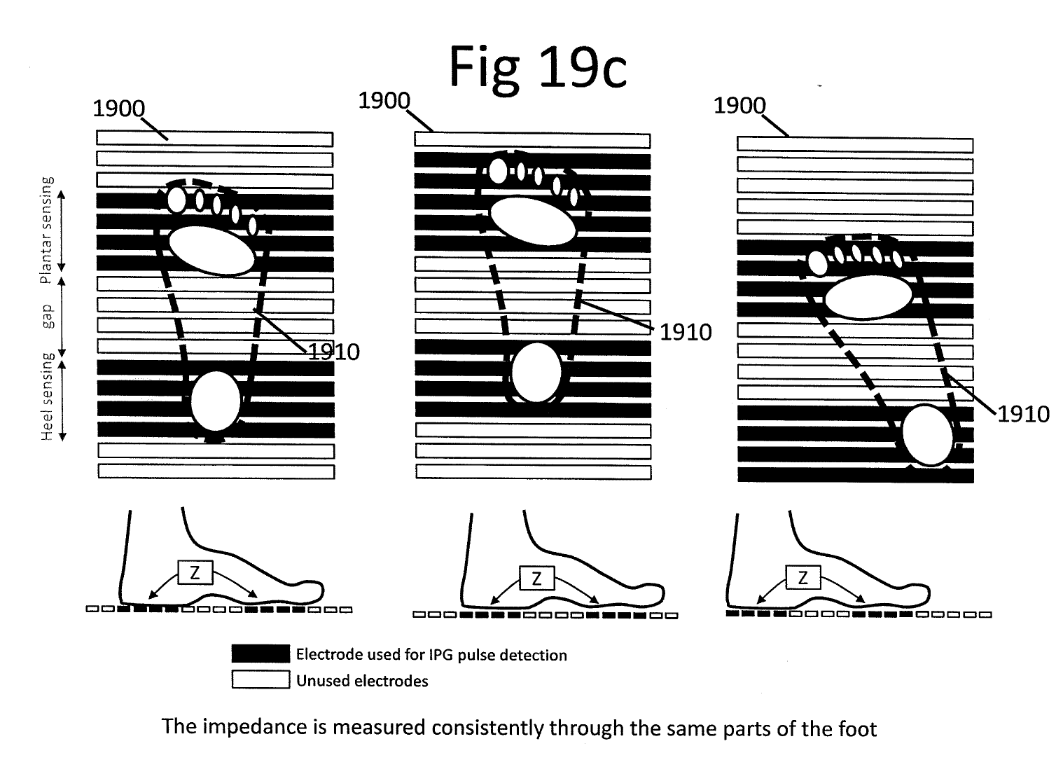

FIGS. 19a-c show example impedance as measured through different parts of the foot based on the foot position, consistent with various aspects of the present disclosure. For instance, example impedance measurement configurations may be implemented using a dynamic electrode configuration for measurement of foot impedance and related timings. Dynamic electrode configuration may be implemented using independently-configurable electrodes to optimize the impedance measurement. As shown in FIG. 19a, interleaved electrodes 1900 are connected to an impedance processor circuit 1905 to determine foot length, foot position, and/or foot impedance. As is shown in FIG. 19b, an impedance measurement is determined regardless of foot position 1910 based on measurement of the placement of the foot across the electrodes 1900. This is based in part in the electrodes 1900 that are engaged (blackened) and in contact with the foot (based on the foot position 1910), which is shown in FIG. 19c.

More specifically regarding FIG. 19a, configuration includes connection/de-connection of the individual electrodes 1900 to the impedance processor circuit 1905, their configuration as current-carrying electrodes (injection or return), sense electrodes (positive or negative), or both. The configuration is preset based on user information, or updated at each measurement (dynamic reconfiguration) to optimize a given parameter (impedance SNR, measurement location). The system algorithmically determines which electrodes under the foot to use in order to obtain the highest SNR in the pulse impedance signal. Such optimization algorithm may include iteratively switching configurations and measuring the impedance, and selecting the best suited configuration. Alternatively, the system first, through a sequential impedance measurement between each individual electrode 1900 and another electrode in contact with the body (such as an electrode in electrode pair 205 on the other foot), determine which electrodes are in contact with the foot. By determining the two most apart electrodes, the foot size is determined. Heel location can be determined in this manner, as can other characteristics such as foot arch type. These parameters are used to determine programmatically (in an automated manner by CPU/logic circuitry) which electrodes are selected for current injection and return (and sensing if a Kelvin connection issued) to obtain the best foot IPG.

In various embodiments involving the dynamically reconfigurable electrode array 1900/1905, an electrode array set is selected to measure the same portion/segment of the foot, irrespective of the foot location on the array. FIG. 19b illustrates the case of several foot positions on a static array (a fixed set of electrodes are used for measurement at the heel and plantar/toe areas, with a fixed gap of an inactive electrode or insulating material between them). Depending on the position of the foot, the active electrodes are contacting the foot at different locations, thereby sensing a different volume/segment of the foot. If the IPG is used by itself (e.g., for heart measurement), such discrepancies may be non-consequential. However, if timings derived from the IPG are referred to other timings (e.g., R-wave from the ECG, or specific timing in the BCG), such as for the calculation of a PTT or PWV, the small shifts in IPG timings due to the sensing of slightly different volumes in the foot (e.g., if the foot is not always placed at the same position on the electrodes) can introduce an error in the calculation of the interval. With respect to FIG. 19b, the timing of the peak of the IPG from the foot placement on the right (sensing the toe/plantar region) is later than from the foot placement on the left, which senses more of the heel volume (the pulse reaches first the heel, then the plantar region). Factors influencing the magnitude of these discrepancies include foot shape (flat or not) and foot length.

Various embodiments address challenges relating to foot placement. FIG. 19c shows an example embodiment involving dynamic reconfiguration of the electrodes to reduce such foot placement-induced variations. As an example, by sensing the location of the heel first (as described above), it is possible to activate a subset of electrodes under the heel, and another subset of electrodes separated by a fixed distance (1900). The other electrodes (e.g., unused electrodes) are left disconnected. The sensed volume will therefore be the same, producing consistent timings. The electrode configuration leading to the most consistent results may be informed by the foot impedance, foot length, the type of arch (all of which can be measured by the electrode array as shown above), but also by the user ID (foot information can be stored for each user, then looked up based on automatic user recognition or manual selection (e.g., in a look-up-table stored for each user in a memory circuit accessible by the CPU circuit in the scale).

In certain embodiments, the apparatus measures impedance using a plurality of electrodes contacting one foot and with at least one other electrode (typically many) at a location distal from the foot. The plurality of electrodes (contacting the one foot) is arranged on the platform and in a pattern configured to inject current signals and sense signals in response thereto, for the same segment of the foot so that the timing of the pulse-based measurements does not vary because the user placed the one foot at a slightly different position on the platform or scale. In FIG. 19a, the foot-to-electrode locations for the heel are different locations than that shown in FIGS. 19b and 19c. As this different foot placement can occur from day to day for the user, the timing and related impedance measurements are for the same (internal) segment of the foot. By having the processor circuit inject current and sense responsive signals to first locate the foot on the electrodes (e.g., sensing where positions of the foot's heel plantar regions and/or toes), the pattern of foot-to-electrode locations permits the foot to move laterally, horizontally and both laterally and horizontally via the different electrode locations, while collecting impedance measurements relative to the same segment of the foot.

The BCG/IPG system can be used to determine the PTT of the user, by identification of the average I-Wave or derivative timing near the I-Wave from a plurality of BCG heartbeat signals obtained simultaneously with the Dual-IPG measurements of the present disclosure to determine the relative PTT along an arterial segment between the ascending aortic arch and distal pulse timing of the user's lower extremity. In certain embodiments, the BCG/IPG system is used to determine the PWV of the user, by identification of the characteristic length representing the length of the user's arteries, and by identification of the average I-Wave or derivative timing near the I-Wave from a plurality of BCG heartbeat signals obtained simultaneously with the Dual-IPG measurements of the present disclosure to determine the relative PTT along an arterial segment between the ascending aortic arch and distal pulse timing of the user's lower extremity. The system of the present disclosure and alternate embodiments may be suitable for determining the arterial stiffness (or arterial compliance) and/or cardiovascular risk of the user regardless of the position of the user's feet within the bounds of the interleaved electrodes. In certain embodiments, the weighing scale system incorporated the use of strain gage load cells and six or eight electrodes to measure a plurality of signals including: bodyweight, BCG, body mass index, fat percentage, muscle mass percentage, and body water percentage, heart rate, heart rate variability, PTT, and PWV measured simultaneously or synchronously when the user stands on the scale to provide a comprehensive analysis of the health and wellness of the user.

In other certain embodiments, the PTT and PWV are computed using timings from the Leg IPG or Foot IPG for arrival times, and using timings from a sensor located on the upper body (as opposed to the scale measuring the BCG) to detect the start of the pulse. Such sensor may include an impedance sensor for impedance cardiography, a hand-to-hand impedance sensor, a photoplethysmogram on the chest, neck, head, arms or hands, or an accelerometer on the chest (seismocardiograph) or head.

Communication of the biometric information is another aspect of the present disclosure. The biometric results from the user are stored in the memory on the scale and displayed to the user via a display on the scale, audible communication from the scale, and/or the data is communicated to a peripheral device such as a computer, smart phone, and tablet computing device. The communication occurs to the peripheral device with a wired connection, or can be sent to the peripheral device through wireless communication protocols such as Bluetooth or WiFi. Computations such as signal analyses described therein may be carried out locally on the scale, in a smartphone or computer, or in a remote processor (cloud computing).

Other aspects of the present disclosure are directed toward apparatuses or methods that include the use of at least two electrodes that contacts feet of a user. Further, circuitry is provided to determine a pulse arrival time at the foot based on the recording of two or more impedance signals from the set of electrodes. Additionally, a second set of circuitry is provided to extract a first pulse arrival time from a first impedance signal and use the first pulse arrival time as a timing reference to extract and process a second pulse arrival time in a second impedance signal.

Various embodiments are implemented in accordance with, and fully incorporating by reference for their general teachings, the above-identified PCT Applications and U.S. Provisional applications (including PCT Ser. No. PCT/US2016/062484 and PCT Ser. No. PCT/US2016/062505), which teachings are also incorporated by reference specifically concerning physiological scales and related measurements and communications such as exemplified by disclosure in connection with FIGS. 1a, 1b, 1e, 1f, 1g, 1j and 2b-2e in PCT Ser. No. PCT/US2016/062484 and FIGS. 1a, 1k, and 1m in PCT. Ser. No. PCT/US2016/062505, and related disclosure in the above-identified U.S. Provisional applications. For example, above-identified U.S. Provisional Application (Ser. No. 62/258,238), which teachings are also incorporated by reference specifically concerning obtaining derivation data, assessing a condition or treatment of the user, and drug titration features and aspects as exemplified by disclosure in connection with FIGS. 1a-1b of the underlying provisional; U.S. Provisional Application (Ser. No. 62/263,385, which teachings are also incorporated by reference specifically to dual authorization of communication between a scale and other devices and biometrics features and aspects as described in connection with FIGS. 1a-1c in the underlying provisional; and U.S. Provisional Application (Ser. No. 62/266,523), which teachings are also incorporated by reference specifically concerning grouping users into inter and intra scale social groups based on aggregated user data sets, and providing normalized user data to other users in the social group aspects as exemplified by disclosure in connection with FIGS. 1a-1c of the underlying provisional. For instance, embodiments herein and/or in the PCT and/or provisional applications may be combined in varying degrees (including wholly). Reference may also be made to the experimental teachings and underlying references provided in the PCT and/or provisional applications.

Embodiments discussed in the provisional applicants are not intended, in any way, to be limiting to the overall technical disclosure, or to any part of the claimed invention unless specifically noted.

Reference may also be made to published patent documents U.S. Patent Publication 2010/0094147 and U.S. Patent Publication 2013/0310700, which are, together with the references cited therein, herein fully incorporated by reference for the purposes of sensors and sensing technology. The aspects discussed therein may be implemented in connection with one or more of embodiments and implementations of the present disclosure (as well as with those shown in the figures). In view of the description herein, those skilled in the art will recognize that many changes may be made thereto without departing from the spirit and scope of the present disclosure.

As illustrated herein, various circuit-based building blocks and/or modules may be implemented to carry out one or more of the operations/activities described herein shown in the block-diagram-type figures. In such contexts, these building blocks and/or modules represent circuits that carry out these or related operations/activities. For example, in certain embodiments discussed above (such as the pulse circuitry modularized as shown in FIGS. 3a-b), one or more blocks/modules are discrete logic circuits or programmable logic circuits for implementing these operations/activities, as in the circuit blocks/modules shown. In certain embodiments, the programmable circuit is one or more computer circuits programmed to execute a set (or sets) of instructions (and/or configuration data). The instructions (and/or configuration data) can be in the form of firmware or software stored in and accessible from a memory circuit. As an example, first and second modules/blocks include a combination of a CPU hardware-based circuit and a set of instructions in the form of firmware, where the first module/block includes a first CPU hardware circuit with one set of instructions and the second module/block includes a second CPU hardware circuit with another set of instructions.

Based upon the above discussion and illustrations, those skilled in the art will readily recognize that various modifications and changes may be made to the present disclosure without strictly following the exemplary embodiments and applications illustrated and described herein. For example, the input terminals as shown and discussed may be replaced with terminals of different arrangements, and different types and numbers of input configurations (e.g., involving different types of input circuits and related connectivity). Such modifications do not depart from the true spirit and scope of the present disclosure, including that set forth in the following claims.

What is claimed is:

1. An apparatus comprising:
a scale comprising:
a platform configured and arranged for a user to stand on,
data-procurement circuitry, including force sensor circuitry and a plurality of electrodes integrated with the platform, and configured and arranged to engage the user with electrical signals and collect signals indicative of the user's identity and cardio-physiological measurements while the user is standing on the platform,
processing circuitry, including a CPU and a memory circuit with user-corresponding data stored in the memory circuit, configured and arranged under the platform upon which the user stands, the processing circuitry being electrically integrated with the force sensor circuitry and the plurality of electrodes and being configured:
to process data obtained by the data-procurement circuitry and therefrom generate cardio-related physiologic data corresponding to the collected signals, and
identify a scale-based biometric of the user using the collected signals, and therefrom, validate user data, including data indicative of the user's identity and the generated cardio-related physiologic data, as concerning the user associated with the scale-based biometric,
a communication activation circuit configured and arranged to activate communication between the scale and a remote user-physiologic device in response identifying the scale-based biometric and verifying authorization data received from the remote user-physiologic device corresponds to the user associated with the scale-based biometric, and
an output circuit configured and arranged to receive the validated user data and, in response, display the user's weight on the user display and output at least a portion of the user data to the remote user-physiologic device responsive to the activated communication.

2. The apparatus of claim 1, further including the remote user-physiologic device, including an output circuit and sensor circuitry, configured and arranged to collect signals indicative of the user's identity, including the authorization data, and cardio-physiological data, and output the authorization data to the scale.

3. The apparatus of claim 1, wherein the communication activation circuitry includes an AND gate configured and arranged to activate communication between the scale and the remote user-physiologic device in response to receiving both the identified scale-based biometric from the processing circuitry and the authorization data from the remote user-physiologic device, and verifying both the scale-based biometric and the authorization data corresponding to the user.

4. The apparatus of claim 1, wherein the communication activation circuitry configured and arranged to activate communication between the scale and the remote user-physiologic device further includes pairing the scale and the remote user-physiologic device via a bi-directional communication.

5. The apparatus of claim 1, further including the remote user-physiologic device, including an output circuit and sensor circuitry, wherein the authorization data includes a remote user-physiological device-based biometric of the user, including biometrics selected from the group consisting of: a finger print, voice recognition, facial recognition, DNA, iris recognition, typing rhythm, and a combination thereof.

6. The apparatus of claim 1, wherein the authorization data includes data selected from the group consisting of a password, a passcode, a biometric, a cellphone ID, and a combination thereof.

7. The apparatus of claim 1, wherein the scale-based biometric of the user include biometrics selected from the group consisting of: foot length, foot width, weight, voice recognition, facial recognition, and a combination thereof.

8. The apparatus of claim 1, further including the remote user-physiologic device, including an output circuit and sensor circuitry, wherein the remote user-physiologic device is a device selected from the group consisting of: a cellphone, a smart watch, a tablet, a plethysmogram, a two terminal ECG sensor, and a combination thereof.

9. The apparatus of claim 2, wherein the cardio-physiologic data generated by the processing circuitry includes data indicative of a BCG of the user and the cardio-physiologic data generated by the remote user-physiologic device includes data indicative of an ECG of the user.

10. The apparatus of claim 2, wherein the remote user-physiologic device includes processing circuitry including a CPU and a memory circuit, and is configured and arranged to correlate the user data from the scale with signals collected by the remote user-physiologic device and to store the correlated user data and collected signals within a user profile corresponding to the user.

11. The apparatus of claim 2, wherein the remote user-physiologic device, including the processing circuitry and sensor circuitry, is further configured and arranged to identity the authorization data of the user using the collected signals indicative of the user's identity, and therefrom, validate the collected signals as concerning the specific user associated with the authorization data.

12. A method comprising:
transitioning a scale, in response to a user standing on a platform of the scale, from a reduced power-consumption mode of operation to at least one higher power-consumption mode of operation, the scale including,
a user display configured and arranged to display data to the user while the user is standing on the scale,
a platform configured and arranged for the user to stand on,
data-procurement circuitry, including force sensor circuitry and a plurality of electrodes integrated with the platform;
processing circuitry, including a CPU and a memory circuit with user-corresponding data stored in the memory circuit, configured and arranged within the scale and under the platform upon which the user stands, the processing circuit being electrically integrated with the force sensor circuitry and the plurality of electrodes; and
a communication activation circuit and an output circuit;
engaging the user with electrical signals, using the data-procurement circuitry, and collecting signals indicative of the user's identity and cardio-physiological measurements while the user is standing on the platform;
processing data, using the processing circuitry, obtained by the data-procurement circuitry while the user is standing on the platform and therefrom generating cardio-related physiologic data corresponding to the collected signals;
identifying, using the processing circuitry, a scale-based biometric of the user using the collected signals, and therefrom, validate user data, including data indicative of the user's identity and the generated cardio-related physiologic data, as concerning the user associated with the scale-based biometric;
activating, using the communication activation circuit, communication between the scale and a remote user-physiologic device in response to the identified scale-based biometric and authorization data corresponding to the user received from the remote user-physiologic device; and
receiving the validated user data and, in response, displaying on the user display the user's weight.

13. The method of claim 12, further including collecting signals, using the remote user-physiologic device, indicative of the user's identity, including the authorization data, and cardio-physiological measurements and outputting the authorization data to the scale.

14. The method of claim 12, wherein the activation of the communication further includes determining reception, via the communication activation circuitry, each of the scale-based biometric and the authentication data within a threshold period of time and:
responsive to receiving both the scale-based biometric and the authentication data within the threshold period of time, activating the communication in response to receiving; and
responsive to receiving at least one of the scale-based biometric and the authentication data outside the threshold period of time, triggers at least one of the processing circuitry of the scale and the remote-physiological device to resend the scale-based biometric and the authentication data.

15. A method and/or apparatus as is consistent with claim 1, in accordance with one or more of the embodiments disclosed herein.
